(19) United States
(12) Patent Application Publication
(10) Pub. No.: US 2015/0216171 A1
(43) Pub. Date: Aug. 6, 2015

(54) HERBICIDALLY ACTIVE BICYCLOARYL CARBOXYLIC ACID AMIDES

(71) Applicant: BAYER CROPSCIENCE AG, Monheim (DE)

(72) Inventors: Hartmut Ahrens, Egelsbach (DE); Andreas Van Almsick, Karben (DE); Ralf Braun, Ramberg (DE); Arnim Koehn, Klein-Winternheim (DE); Stefan Lehr, Lyon (FR); Hansjoerg Dietrich, Liederbach (DE); Elmar Gatzweiler, Bad Nauheim (DE); Christopher Hugh Rosinger, Hofheim (DE); Dirk Schmutzler, Hattersheim (DE)

(73) Assignee: BAYER CROPSCIENCE AG, Monheim (DE)

(21) Appl. No.: 14/423,842

(22) PCT Filed: Sep. 3, 2013
(86) PCT No.: PCT/EP2013/068173

§ 371 Date: Feb. 25, 2015

(30) Foreign Application Priority Data
Sep. 5, 2012 (EP) 12183128.3

(51) Int. Cl. A01N 43/82; A01N 43/713; A01N 43/653; C07D 401/12; C07D 257/06; C07D 249/14; C07D 271/08; C07D 409/12; C07D 413/12

(52) U.S. Cl. CPC A01N 43/82; C07D 409/12; A01N 43/713; A01N 43/653; C07D 413/12; C07D 257/06; C07D 249/14; C07D 271/08; C07D 401/12

(57) ABSTRACT

Bicycloarylcarboxamides of the general formula (I) are described as herbicides.

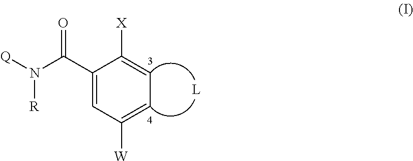

In this formula (I), X and W are radicals such as hydrogen, organic radicals such as alkyl, and other radicals such as halogen. Q is triazolyl, tetrazolyl or oxadiazolyl.

The invention relates to the technical field of herbicides, especially that of herbicides for the selective control of broad-leaved weeds and weed grasses in crops of useful plants.

European patent application EP 11158258, which has an earlier priority date but was yet to be published at the priority date of the present application, discloses N-(tetrazol-5-yl)-, N-(triazol-5-yl)- and N-(1,2,5-oxadiazol-3-yl)bicycloarylcarboxamides and the use thereof as herbicides. However, these compounds do not always have sufficient efficacy against harmful plants and/or some of them do not have sufficient compatibility with some important crop plants such as cereal species, corn or rice.

It has now been found that N-(tetrazol-5-yl)-, N-(triazol-5-yl)-, N-(1,2,5-oxadiazol-3-yl)- and N-(1,3,4-oxadiazol-2-yl)bicycloarylcarboxamides which differ from the compounds known from the prior art essentially in that the ring fused onto the phenyl ring is unsaturated are of particularly good suitability as herbicides.

The present invention thus provides N-(tetrazol-5-yl)-, N-(triazol-5-yl)-, N-(1,2,5-oxadiazol-3-yl)- and N-(1,3,4-oxadiazol-2-yl)bicycloarylcarboxamides of the formula (I) or salts thereof

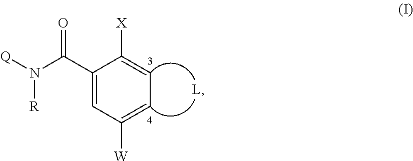

where the symbols and indices are each defined as follows:

Q is a Q1, Q2, Q3 or Q4 radical,

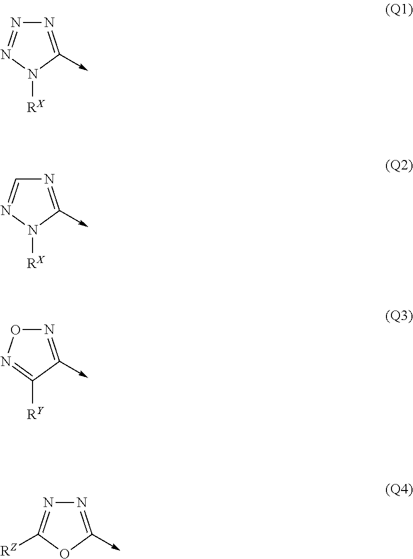

X is nitro, halogen, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R¹O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups,

W is hydrogen, halogen, nitro, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, halo-(C₃-C₇)-cycloalkyl, (C₁-C₆)-alkoxy, halo-(C₁-C₆)-alkoxy, (C₁-C₆)-alkyl-(O)ₙS—, (C₁-C₆)-haloalkyl-(O)ₙS—, (C₁-C₆)-alkoxy-(C₁-C₄)-alkyl, (C₁-C₆)-alkoxy-(C₁-C₄)-haloalkyl, R¹(O)C, R¹(R¹ON═)C, R¹O(O)C, (R¹)₂N, R¹(O)C(R¹)N or R²(O)₂S(R¹)N,

R is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C, R¹O(O)C, (R¹)₂N(O)C, R¹O, (R¹)₂N, R²O(O)C(R¹)N, (R¹)₂N(O)C(R¹)N, R²(O)₂S,

or benzyl substituted in each case by s radicals from the group consisting of methyl, ethyl, methoxy, nitro, trifluoromethyl and halogen,

R^{X} is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of nitro, cyano, (R⁶)₃Si, (R⁵O)₂(O)P, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, and where the four latter radicals are each substituted by s radicals from the group consisting of (C₁-C₆)-alkyl, halo-(C₁-C₆)-alkyl, (C₁-C₆)-alkoxy, halo-(C₁-C₆)-alkoxy and halogen, and

where heterocyclyl bears n oxo groups,

or

R^{X} is (C₃-C₇)-cycloalkyl, heteroaryl, heterocyclyl or phenyl, where the four aforementioned radicals are each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl,

R^{Y} is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, (C₁-C₆)-alkoxy, halo-(C₁-C₆)-alkoxy, (C₂-C₆)-alkenyloxy, (C₂-C₆)-alkynyloxy, cyano, nitro, methylsulfenyl, methylsulfinyl, methylsulfonyl, acetylamino, benzoylamino, methoxycarbonyl, ethoxycarbonyl, methoxycarbonylmethyl, ethoxycarbonylmethyl, benzoyl, methylcarbonyl, piperidinylcarbonyl, trifluoromethylcarbonyl, halogen, amino, aminocarbonyl, methylaminocarbonyl, dimethylaminocarbonyl, methoxymethyl, or heteroaryl, heterocyclyl or phenyl, each of which is substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,

R^{Z} is hydrogen, (C₁-C₆)-alkyl, R¹O—(C₁-C₆)-alkyl, R′H₂, (C₃-C₇)-cycloalkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, R¹O, R¹(H)N, methoxycarbonyl, ethoxycarbonyl, methylcarbonyl, dimethylamino, trifluoromethylcarbonyl, acetylamino, methylsulfenyl, methylsulfinyl, methylsulfonyl, or heteroaryl, heterocyclyl, benzyl oder phenyl each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, where heterocyclyl bears n oxo groups,

R¹ is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl, heterocyclyl-S(O)ₙ—(C₁-C₆)-alkyl, where the fifteen latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl bears n oxo groups,

R² is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl, heterocyclyl-S(O)ₙ—(C₁-C₆)-alkyl, where the fifteen latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl bears n oxo groups,

R³ is hydrogen, (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,

R⁴ is (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,

R⁵ is hydrogen or (C₁-C₄)-alkyl,

R⁶ is (C₁-C₄)-alkyl,

R′ is acetoxy, acetamido, N-methylacetamido, benzoyloxy, benzamido, N-methylbenzamido, methoxycarbonyl, ethoxycarbonyl, benzoyl, methylcarbonyl, piperidinylcarbonyl, morpholinylcarbonyl, trifluoromethylcarbonyl, aminocarbonyl, methylaminocarbonyl, dimethylaminocarbonyl, (C₃-C₆)-cycloalkyl, or heteroaryl or heterocyclyl each substituted by s radicals from the group consisting of methyl, ethyl, methoxy, trifluoromethyl and halogen;

n is 0, 1 or 2,

m is 0, 1, 2, 3 or 4,

s is 0, 1, 2 or 3,

t is 0, 1, 2, 3, 4 or 5,

L is a 3-, 4- or 5-membered fused-on unsaturated bridge wherein the bridge atoms consist of t carbon atoms and m heteroatoms from the group consisting of O, S and N.

In the formula (I) and all the formulae which follow, alkyl radicals having more than two carbon atoms may be straight-chain or branched. Alkyl radicals are, for example, methyl, ethyl, n- or isopropyl, n-, iso-, tert- or 2-butyl, pentyls, and hexyls, such as n-hexyl, isohexyl, and 1,3-dimethylbutyl. Analogously, alkenyl is, for example, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl. Alkynyl is, for example, propargyl, but-2-yn-1-yl, but-3-yn-1-yl, 1-methylbut-3-yn-1-yl. The multiple bond may be in any position in each unsaturated radical. Cycloalkyl is a carbocyclic saturated ring system having three to six carbon atoms, for example cyclopropyl, cyclobutyl, cyclopentyl or cyclohexyl. Analogously, cycloalkenyl is a monocyclic alkenyl group having three to six carbon ring members, for example cyclopropenyl, cyclobutenyl, cyclopentenyl and cyclohexenyl, where the double bond may be in any position.

Halogen is fluorine, chlorine, bromine or iodine.

Heterocyclyl is a saturated, semisaturated or fully unsaturated cyclic radical containing 3 to 6 ring atoms, of which 1 to 4 are from the group consisting of oxygen, nitrogen and sulfur, and which may additionally be fused by a benzo ring. For example, heterocyclyl is piperidinyl, pyrrolidinyl, tetrahydrofuranyl, dihydrofuranyl and oxetanyl.

Heteroaryl is an aromatic cyclic radical containing 3 to 6 ring atoms, of which 1 to 4 are from the group consisting of oxygen, nitrogen and sulfur, and which may additionally be fused by a benzo ring. For example, heteroaryl is benzimidazol-2-yl, furanyl, imidazolyl, isoxazolyl, isothiazolyl, oxazolyl, pyrazinyl, pyrimidinyl, pyridazinyl, pyridinyl, benzisoxazolyl, thiazolyl, pyrrolyl, pyrazolyl, thiophenyl, 1,2,3-oxadiazolyl, 1,2,4-oxadiazolyl, 1,2,5-oxadiazolyl, 1,3,4-oxadiazolyl, 1,2,4-triazolyl, 1,2,3-triazolyl, 1,2,5-triazolyl, 1,3,4-triazolyl, 1,2,4-triazolyl, 1,2,4-thiadiazolyl, 1,3,4-thiadiazolyl, 1,2,3-thiadiazolyl, 1,2,5-thiadiazolyl, 2H-1,2,3,4-tetrazolyl, 1H-1,2,3,4-tetrazolyl, 1,2,3,4-oxatriazolyl, 1,2,3,5-oxatriazolyl, 1,2,3,4-thiatriazolyl and 1,2,3,5-thiatriazolyl.

When a group is polysubstituted by radicals, this means that this group is substituted by one or more identical or different radicals among those mentioned. This applies analogously to the formation of ring systems by various atoms and elements. At the same time, the scope of the claims shall exclude those compounds known by the person skilled in the art to be chemically unstable under standard conditions.

Depending on the nature of the substituents and the way in which they are attached, the compounds of the general formula (I) may be present as stereoisomers. If, for example, one or more asymmetrically substituted carbon atoms are present, there may be enantiomers and diastereomers. There are likewise stereoisomers if sulfoxides are present. Stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries. The invention also relates to all stereoisomers and mixtures thereof which are encompassed by the general formula (I) but not defined specifically.

The compounds of the formula (I) are capable of forming salts. Salts may be formed by action of a base on compounds of the formula (I). Examples of suitable bases are organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺, in which R^{a} to R^{d} are each case independently an organic radical, especially alkyl, aryl, aralkyl or alkylaryl. Also useful are alkylsulfonium and alkylsulfoxonium salts, such as (C₁-C₄)-trialkylsulfonium and (C₁-C₄)-trialkylsulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid, or sulfonic acids, for example p-toluenesulfonic acid, onto a basic group, for example amino, alkylamino, dialkylamino, piperidino, morpholino or pyridino.

Preference is given to compounds of the general formula (I) or the salts thereof in which the L bridge represents the A1 to A378 radicals, where the dotted bonds represent those bonds that bind the L bridge to the benzoyl radical. The upper dotted line here represents the bond to carbon atom 3 in the general formula (I), and the lower dotted line the bond to carbon atom 4 in the general formula (I):

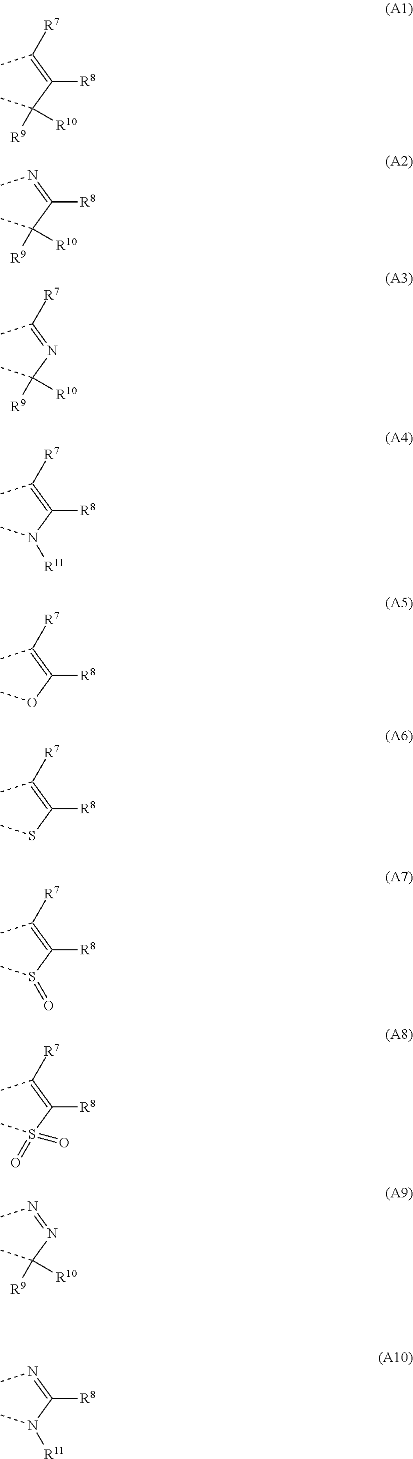

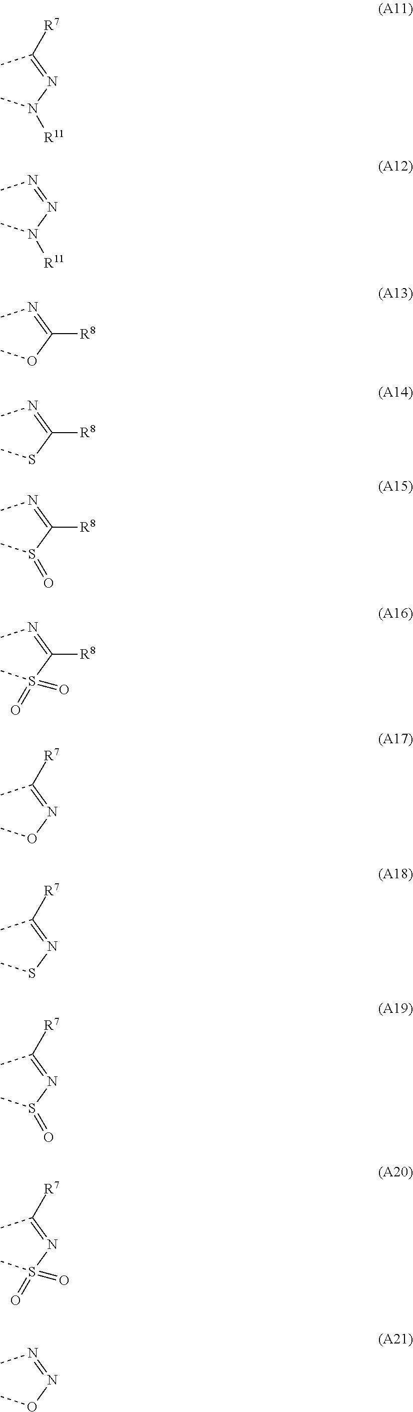

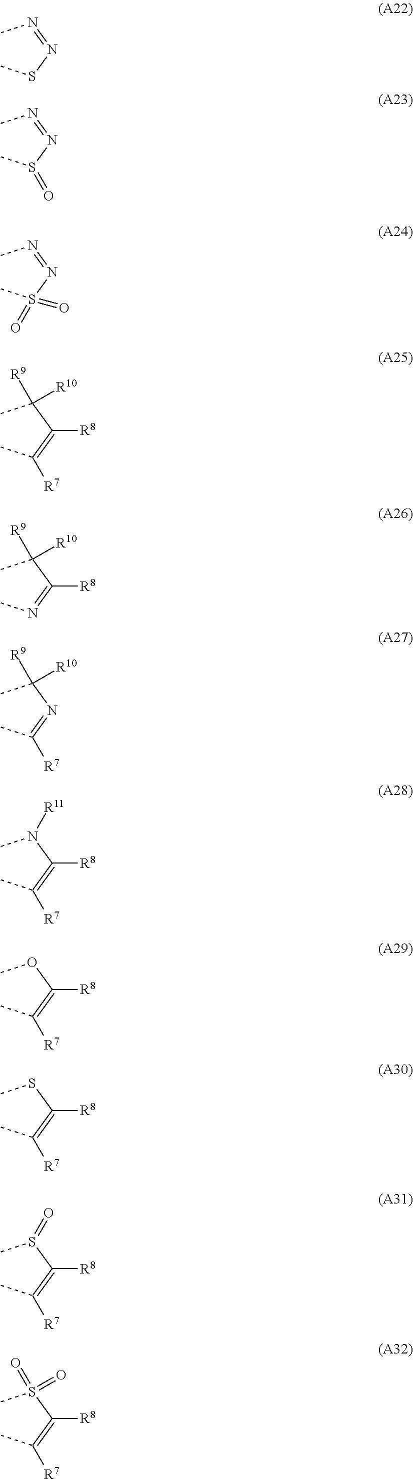

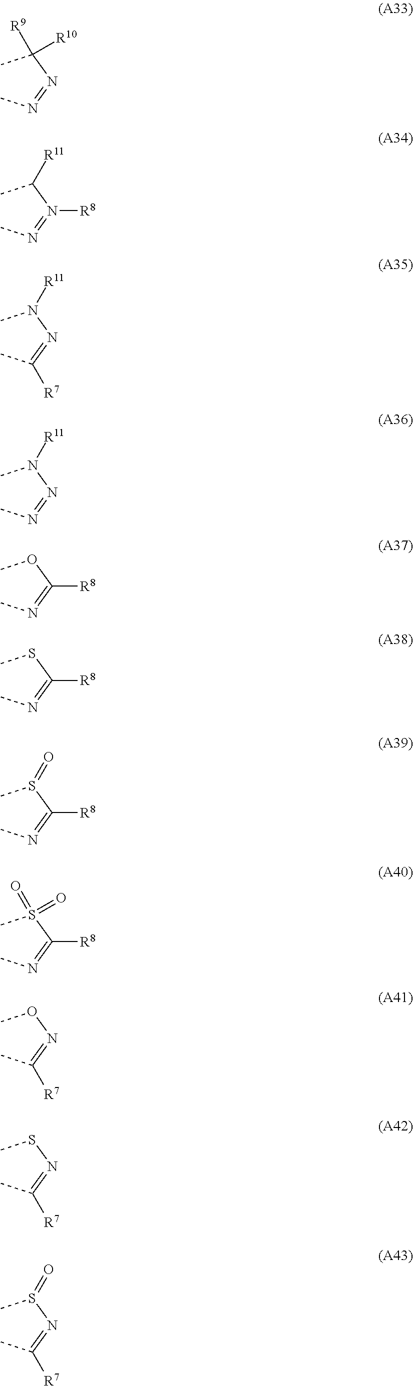

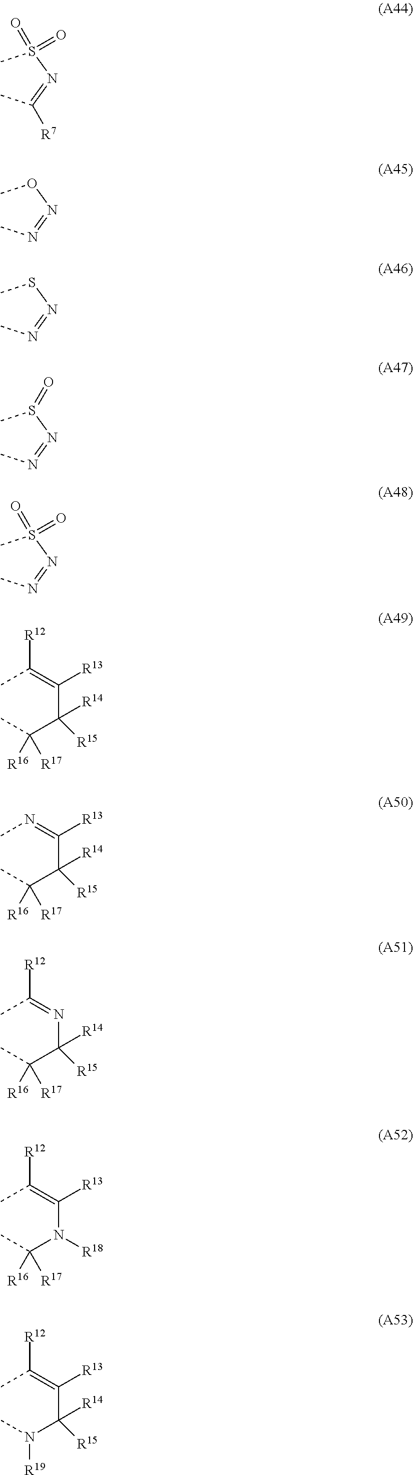

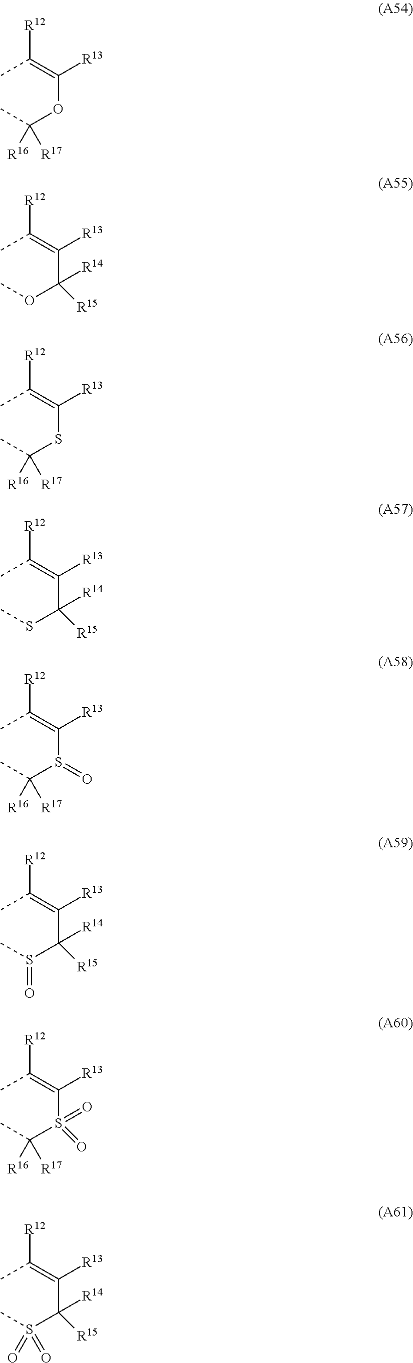

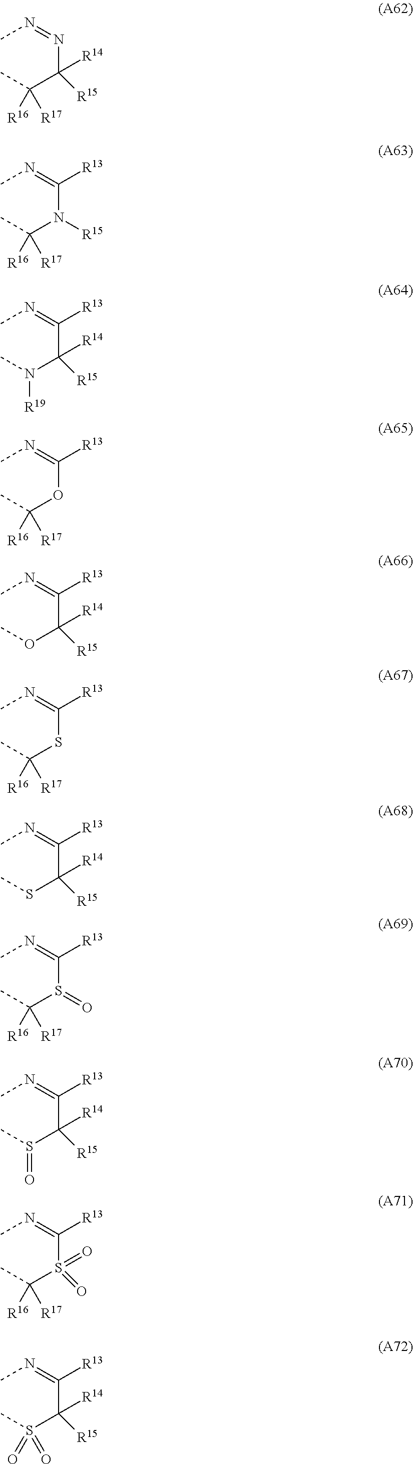

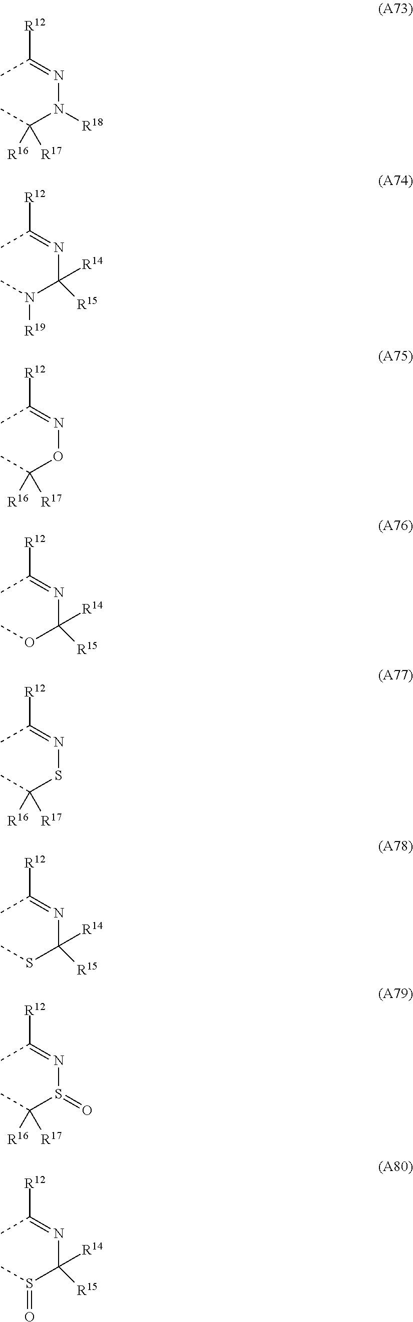

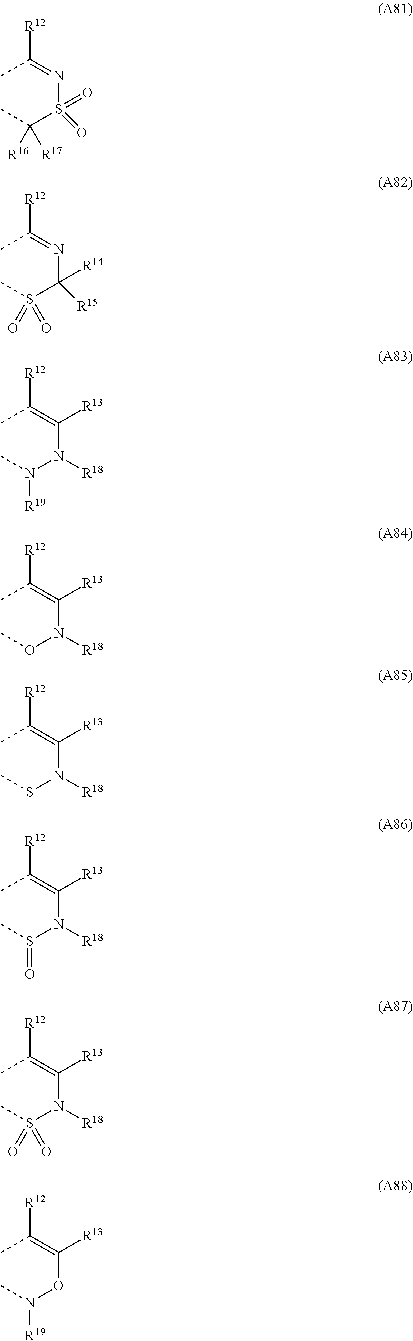

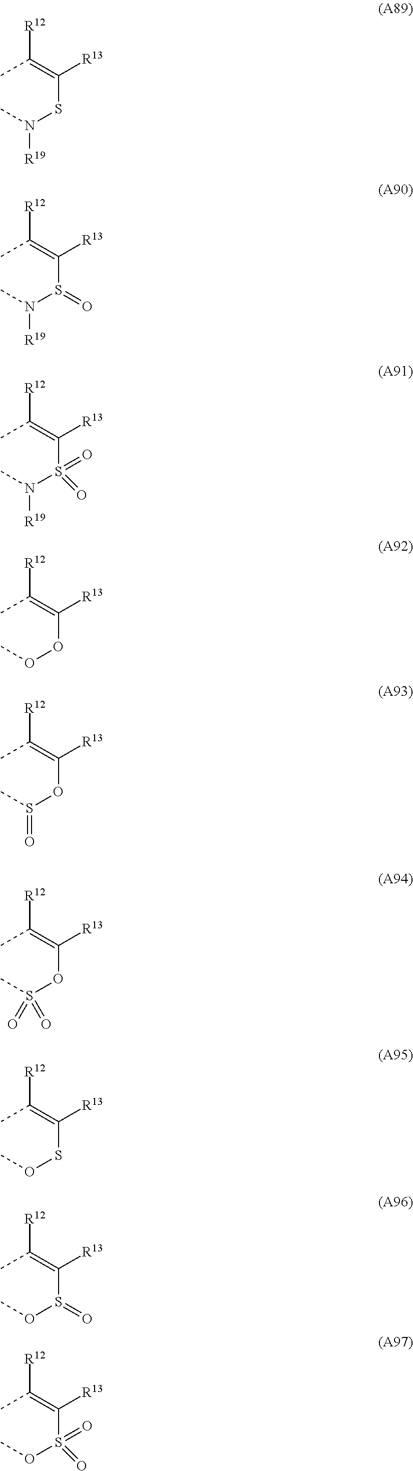

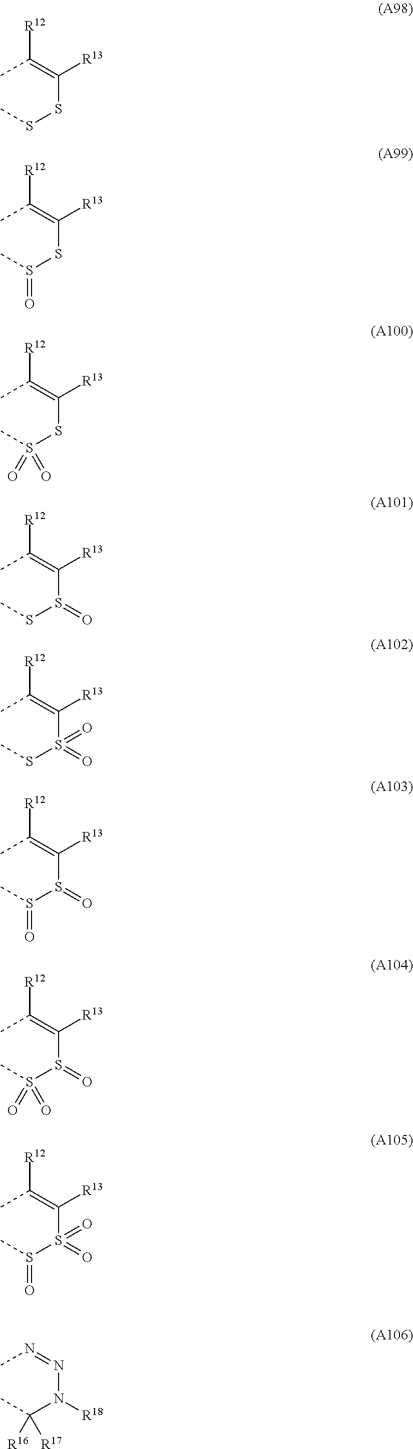

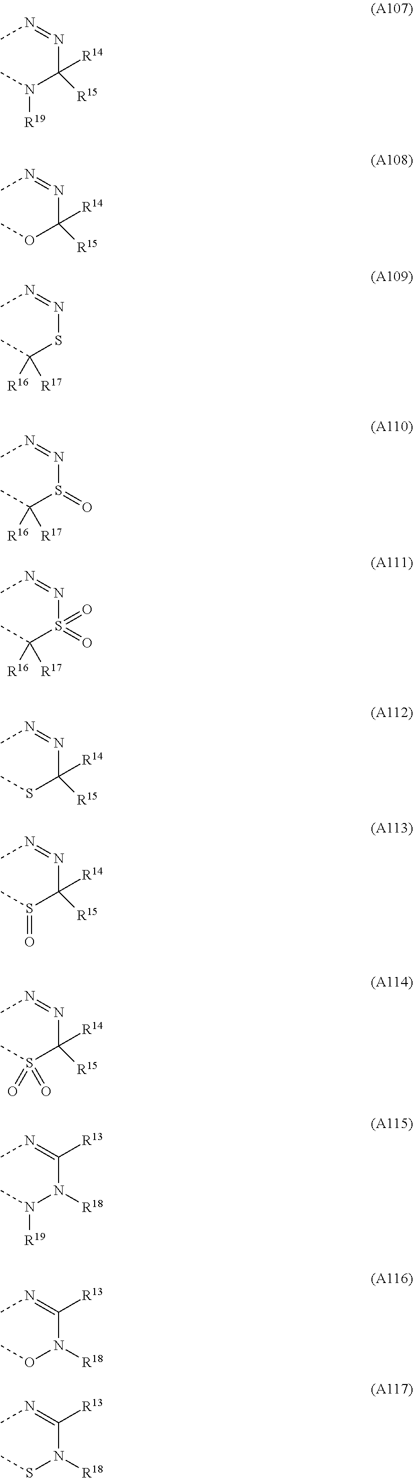

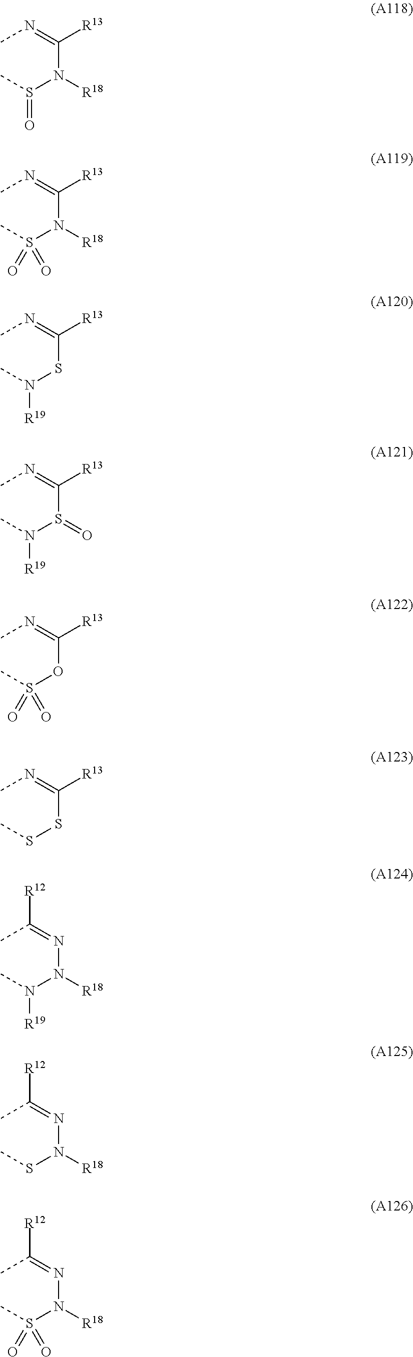

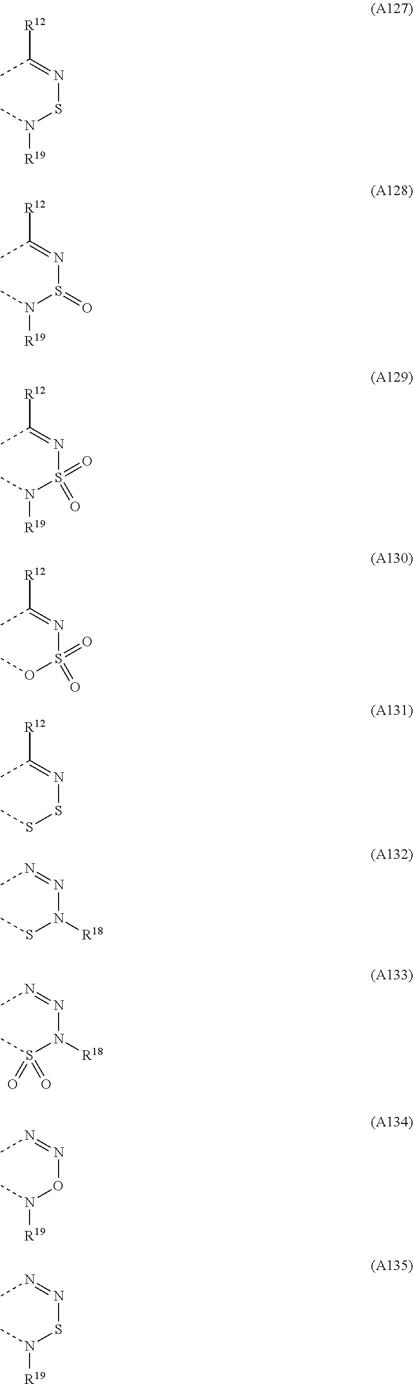

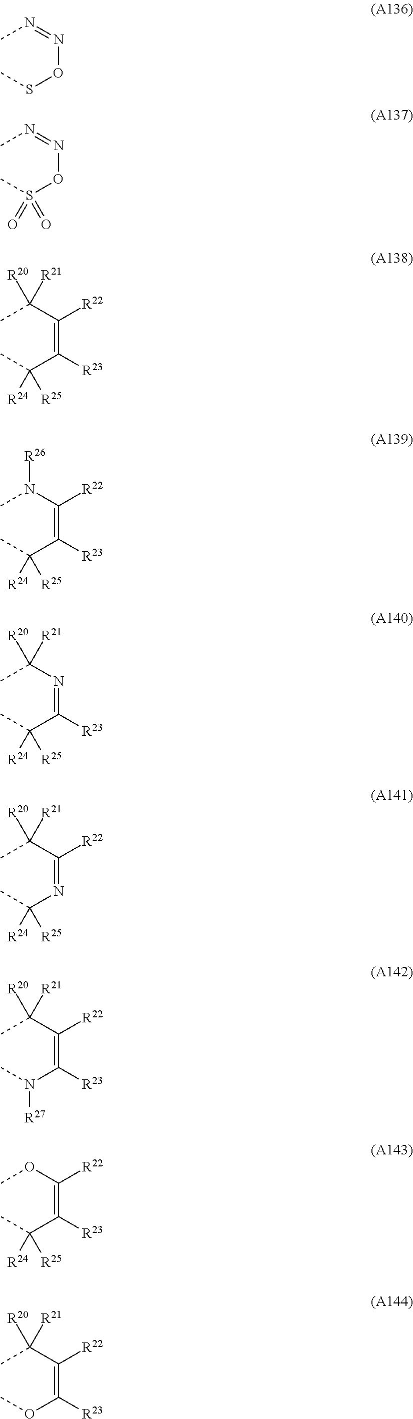

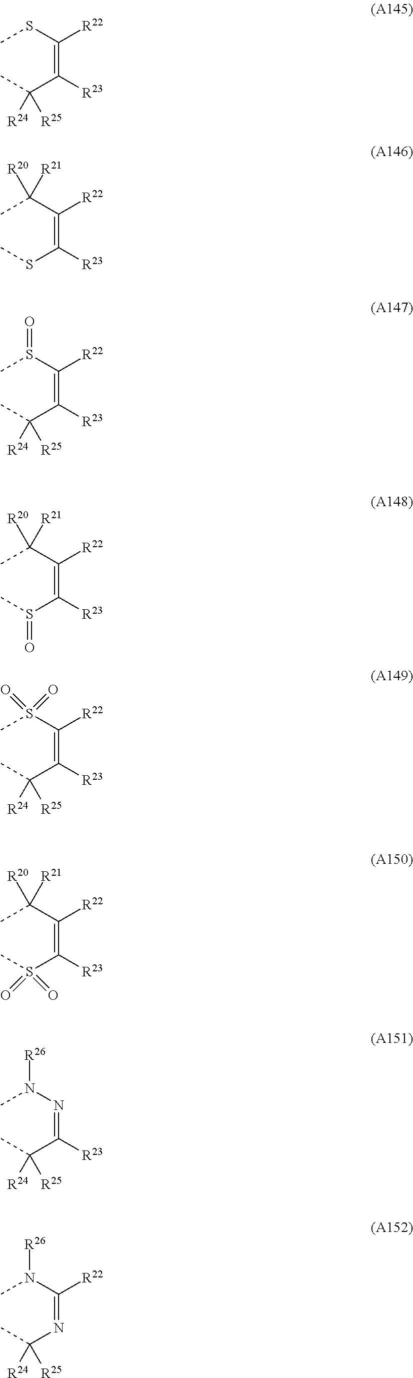

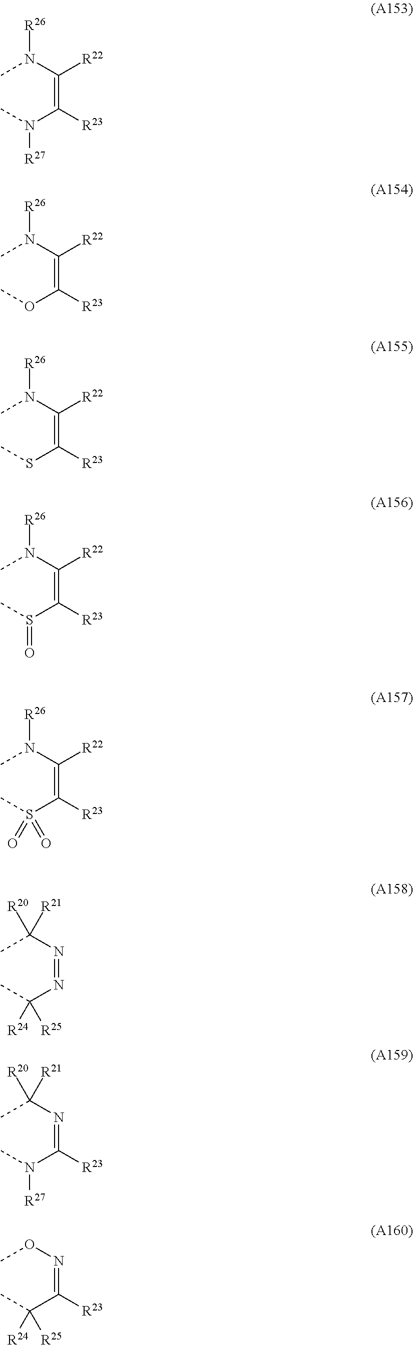

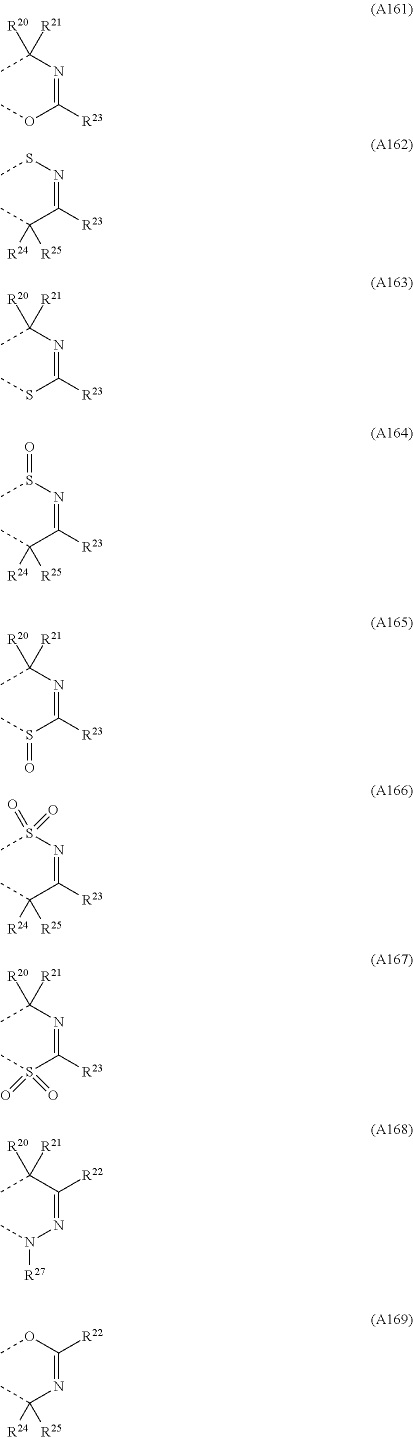

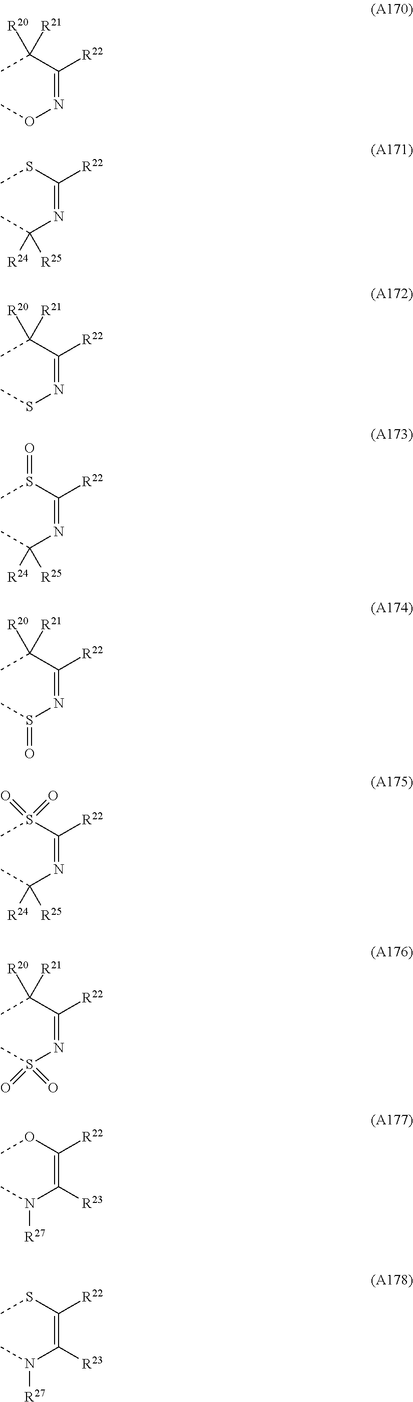

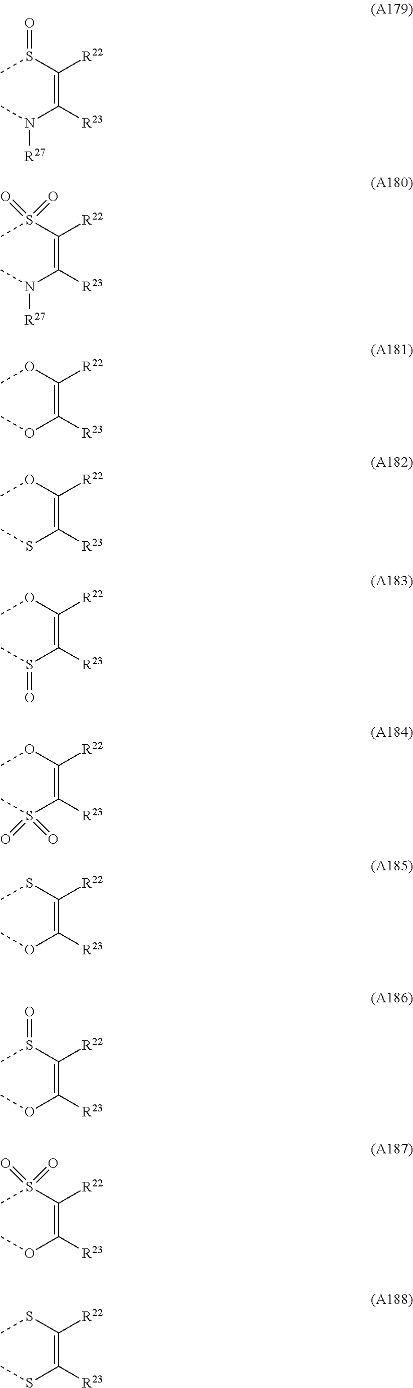

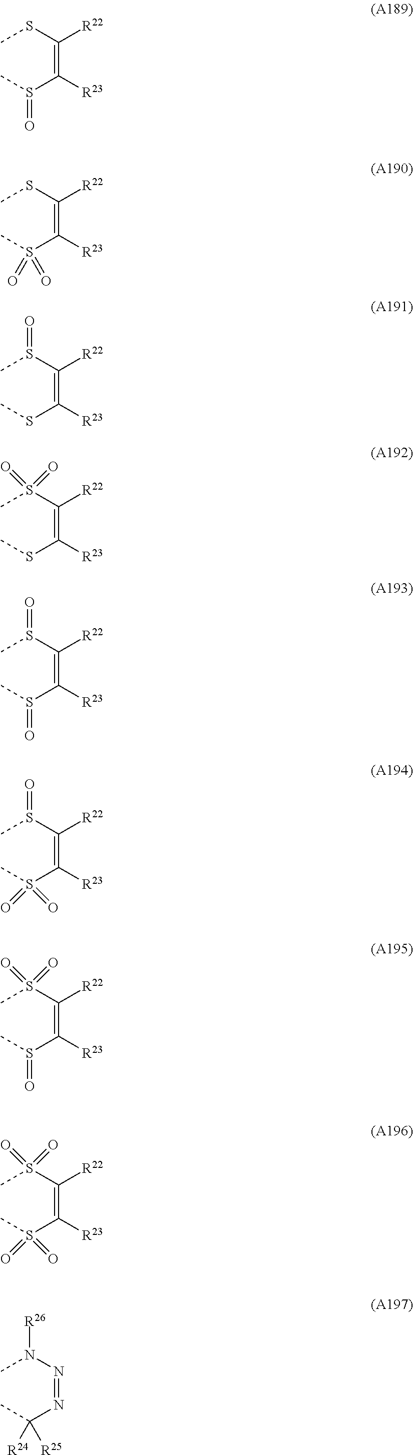

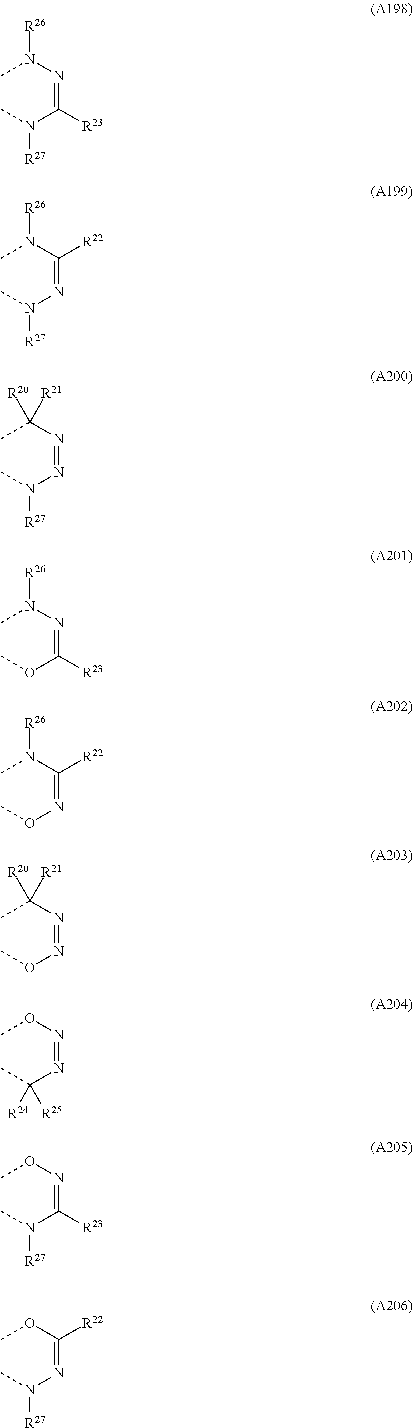

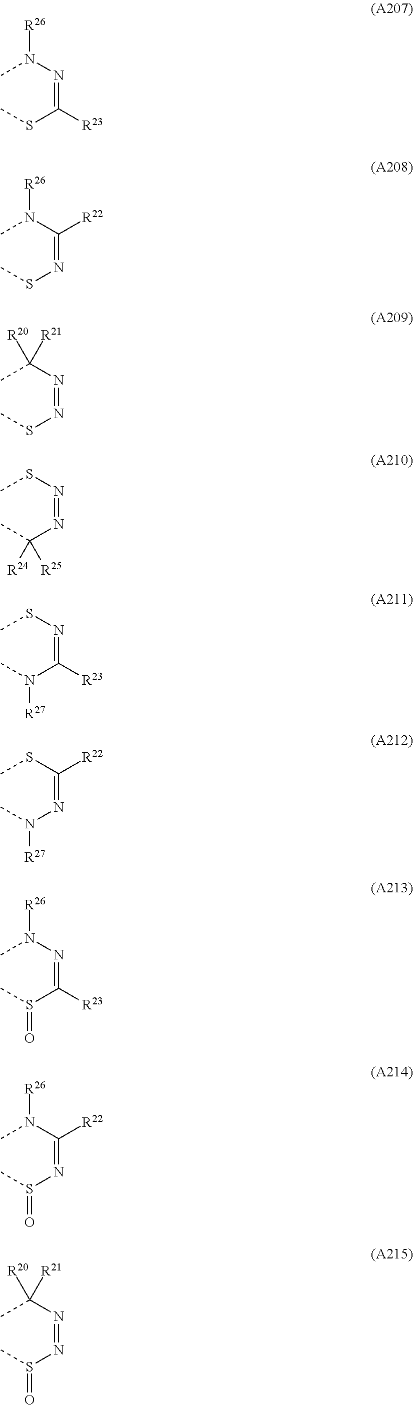

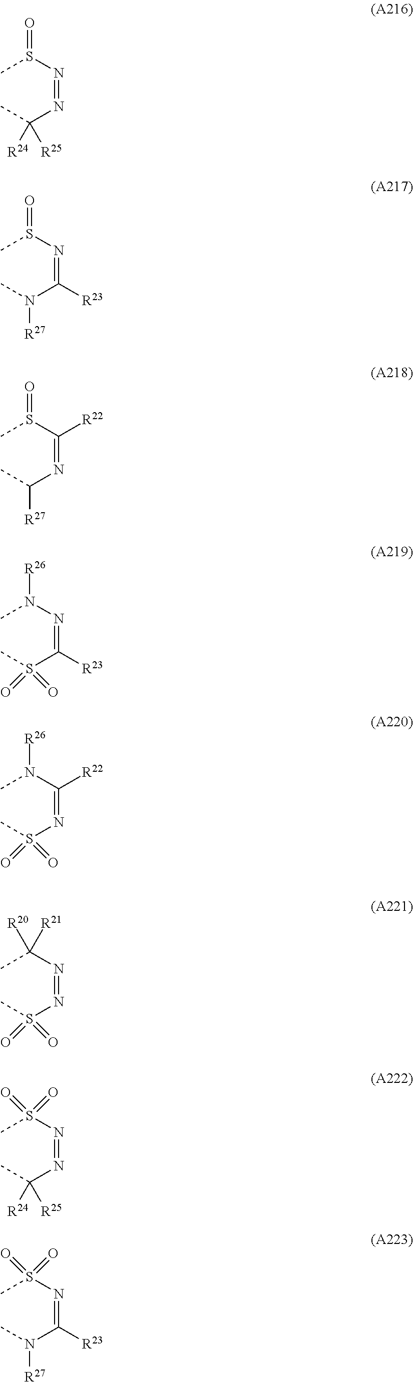

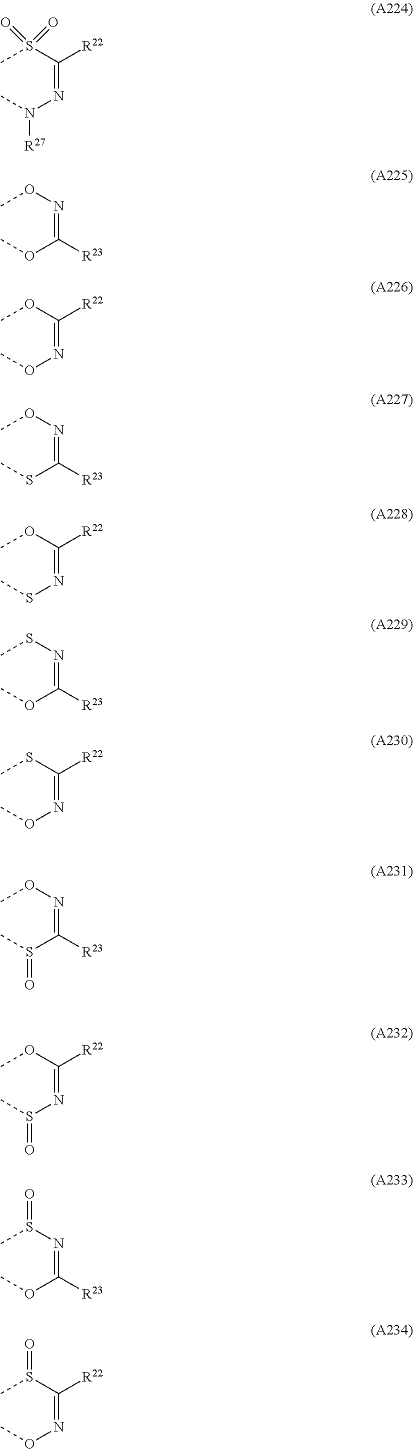

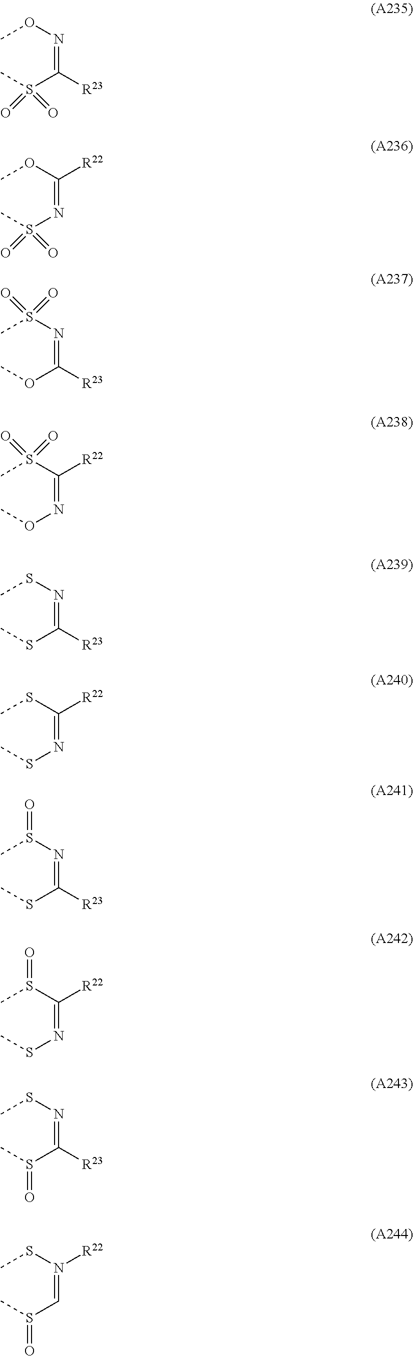

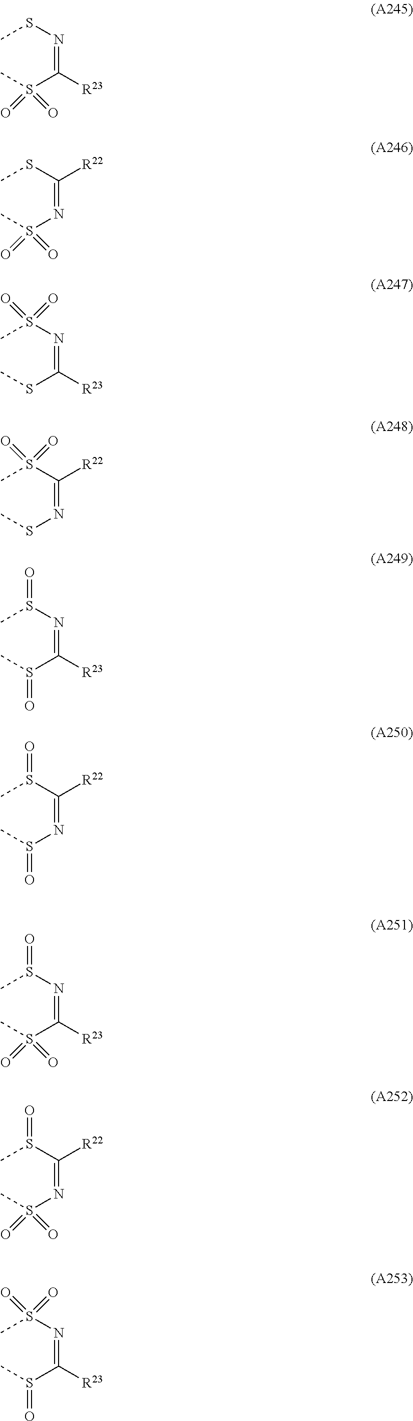

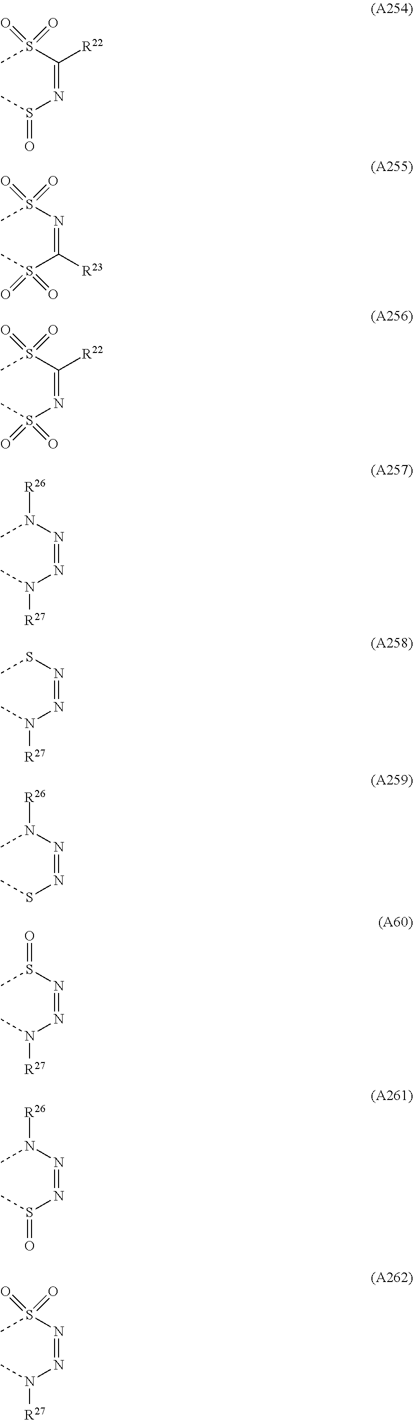

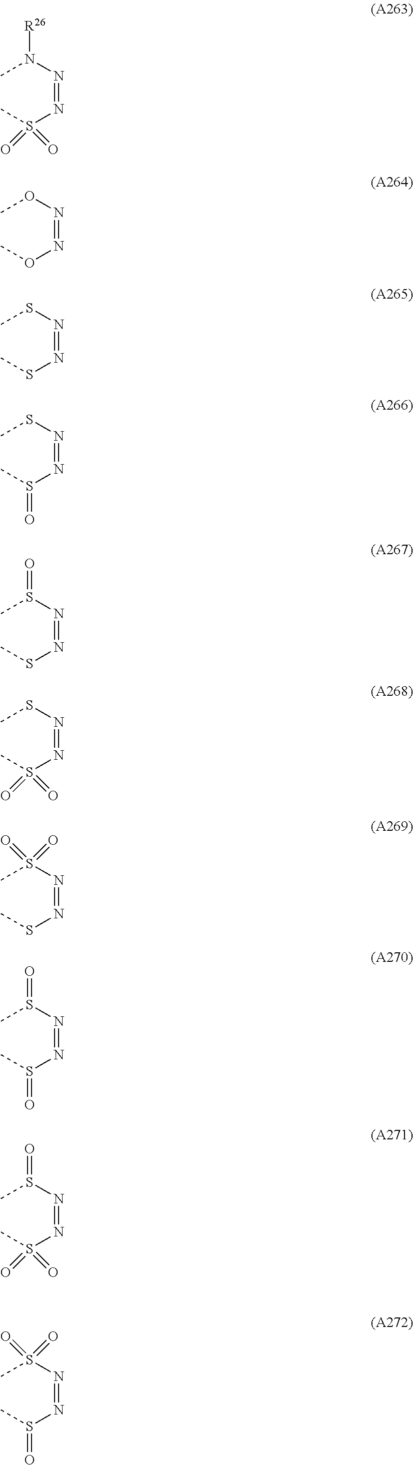

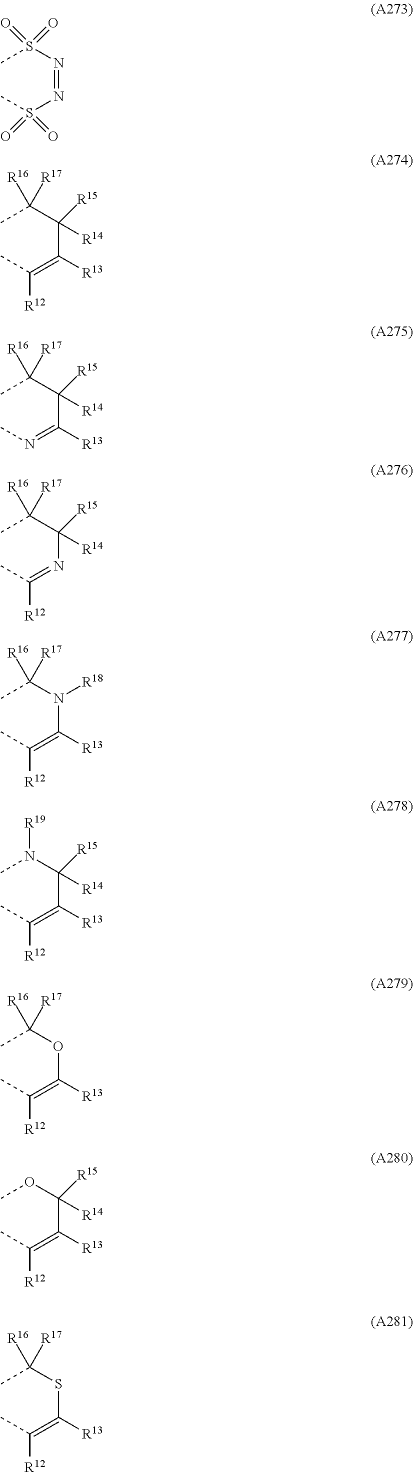

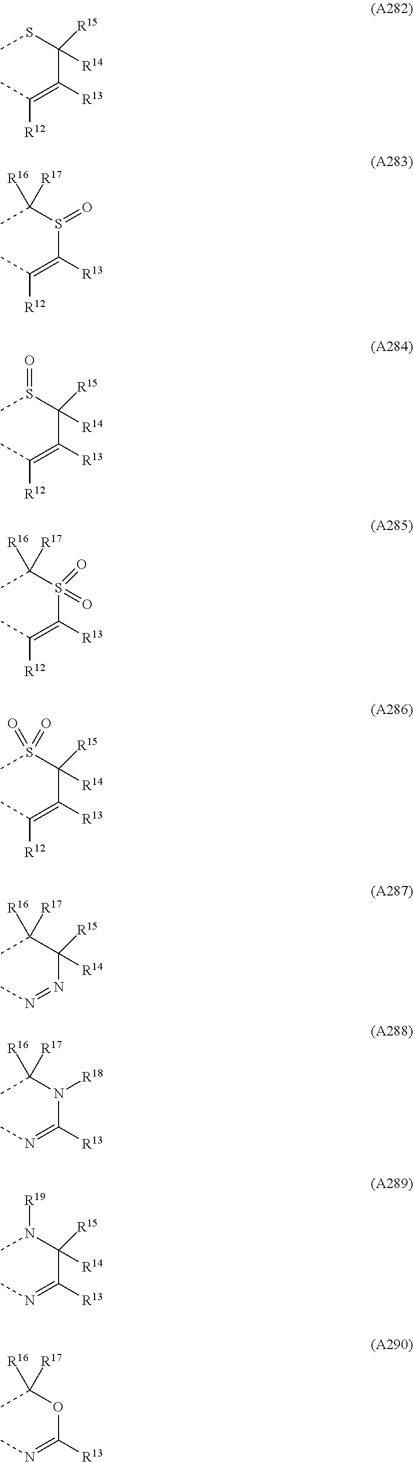

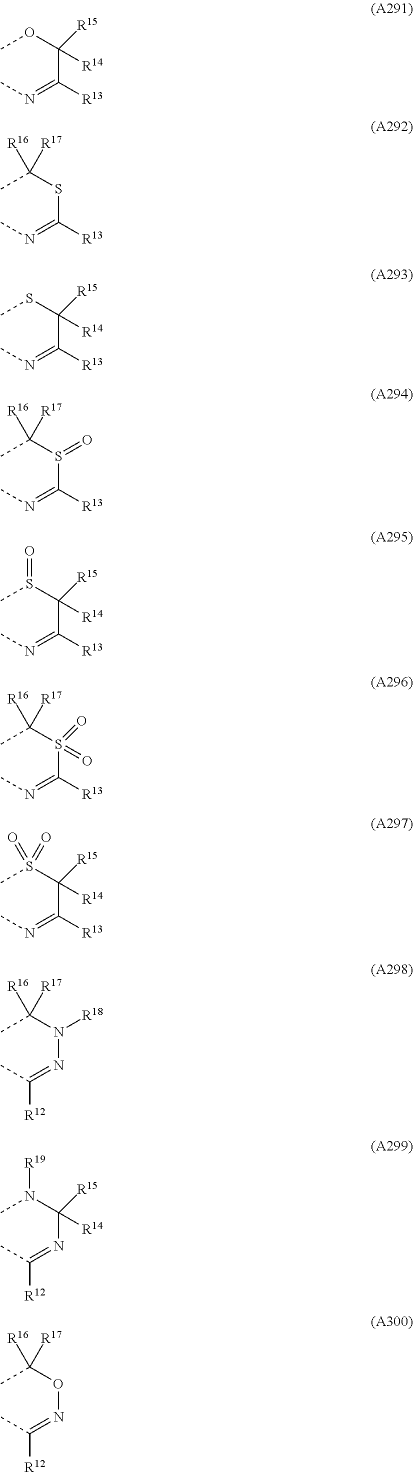

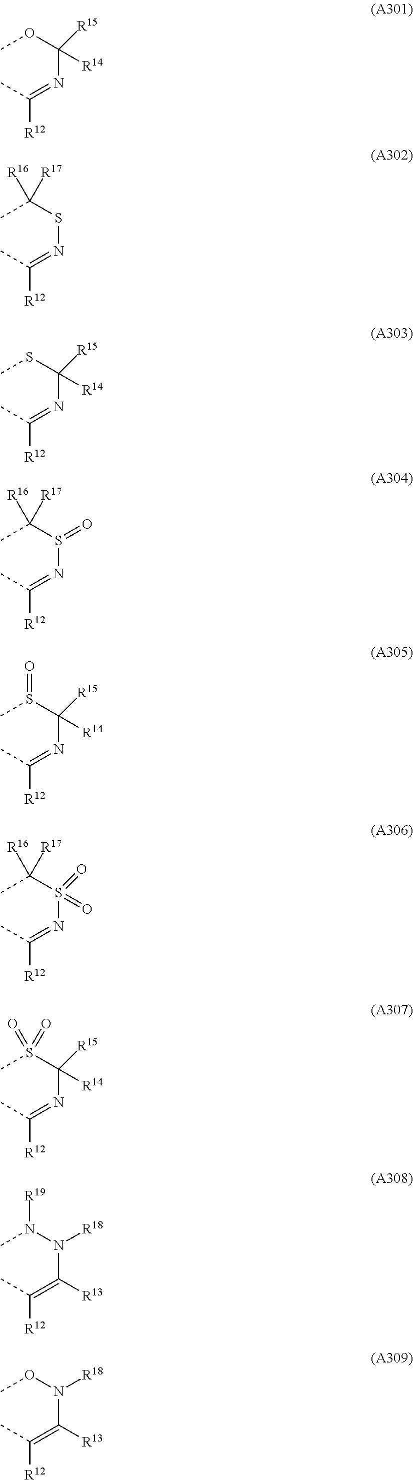

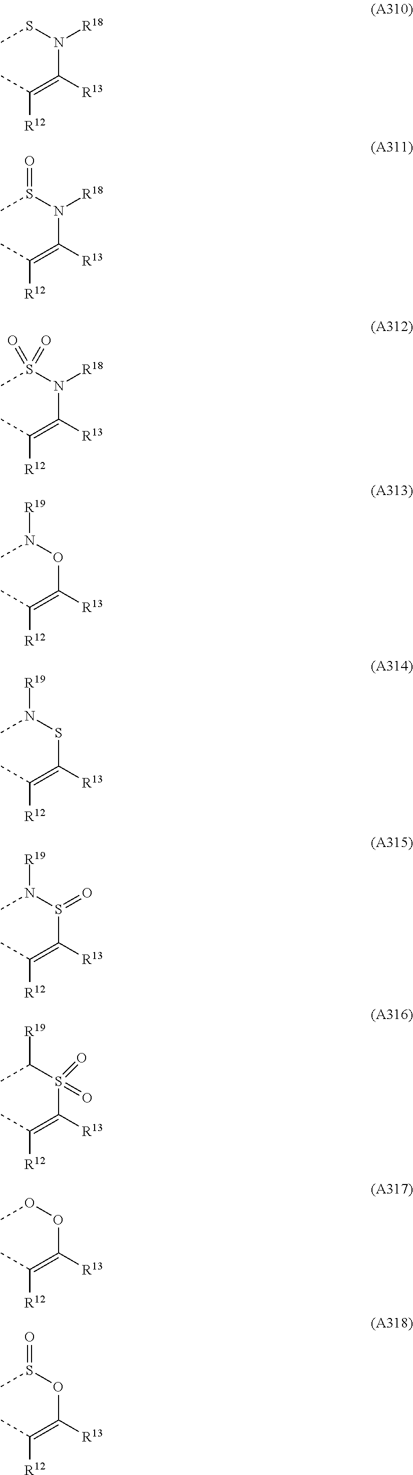

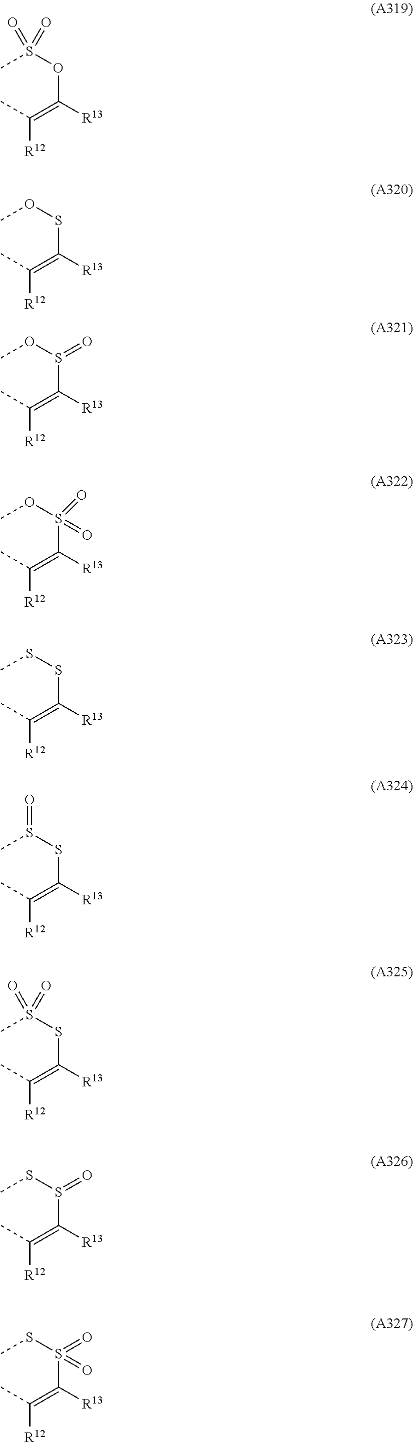

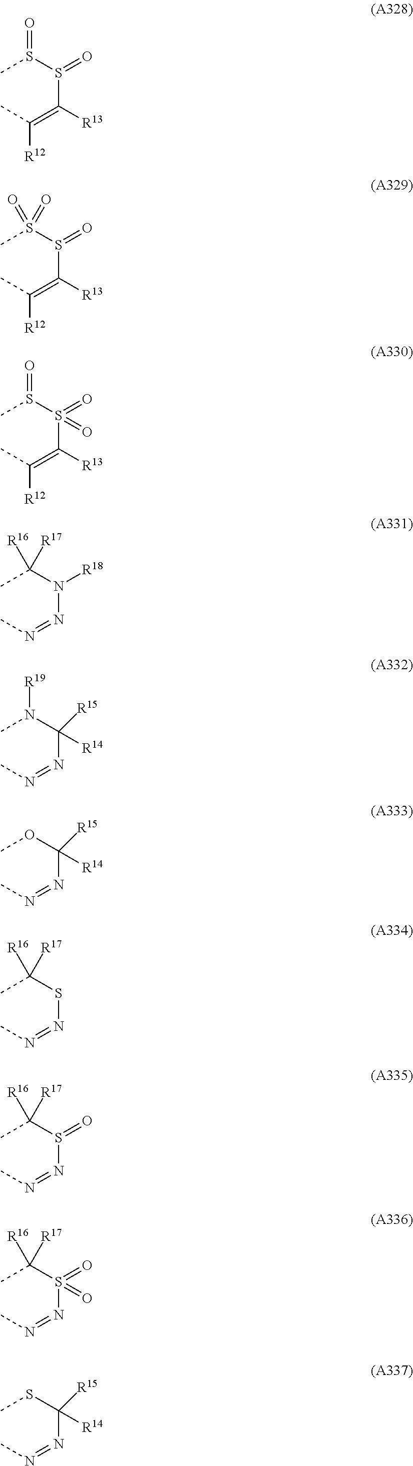

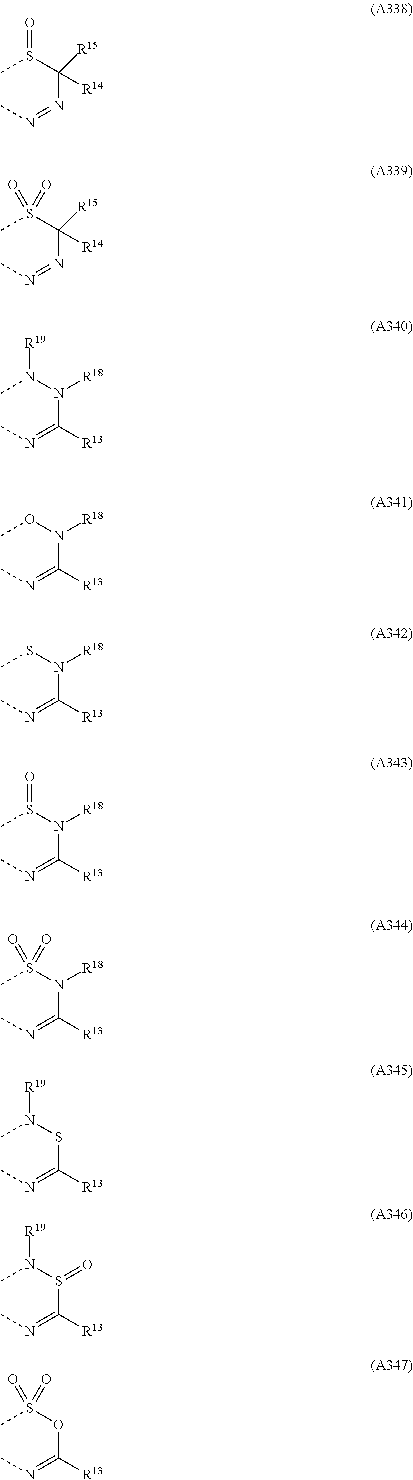

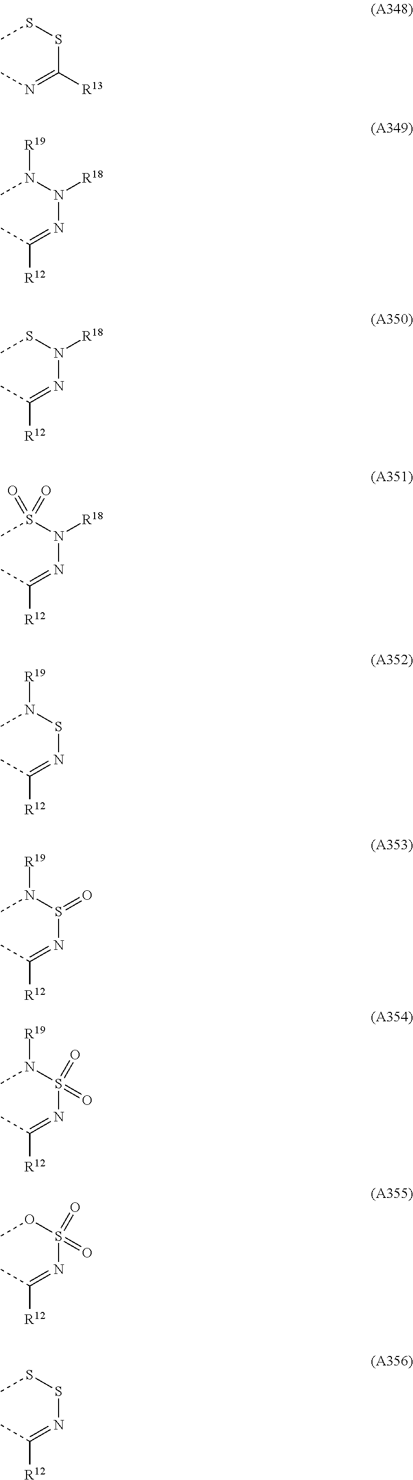

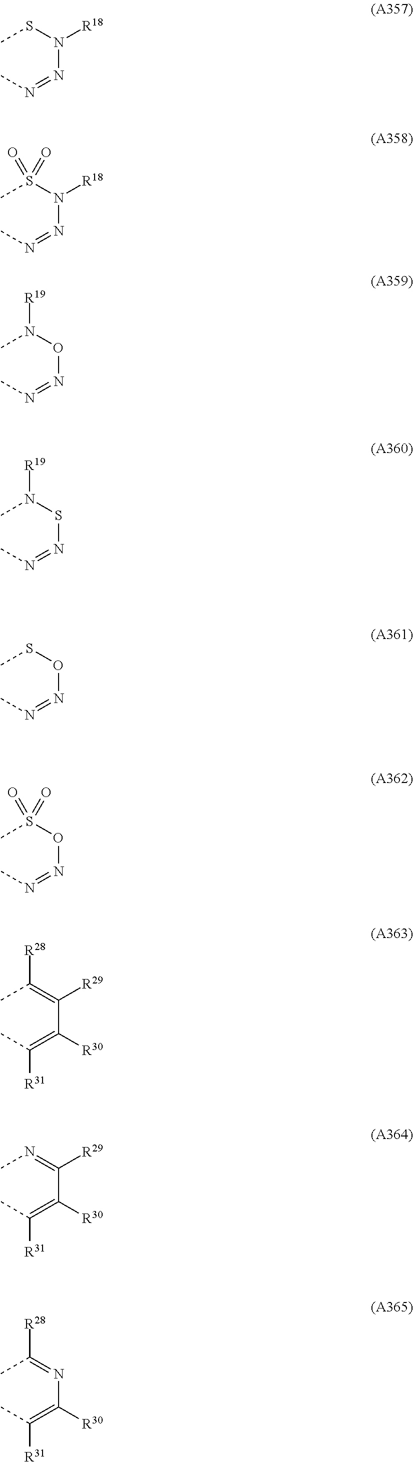

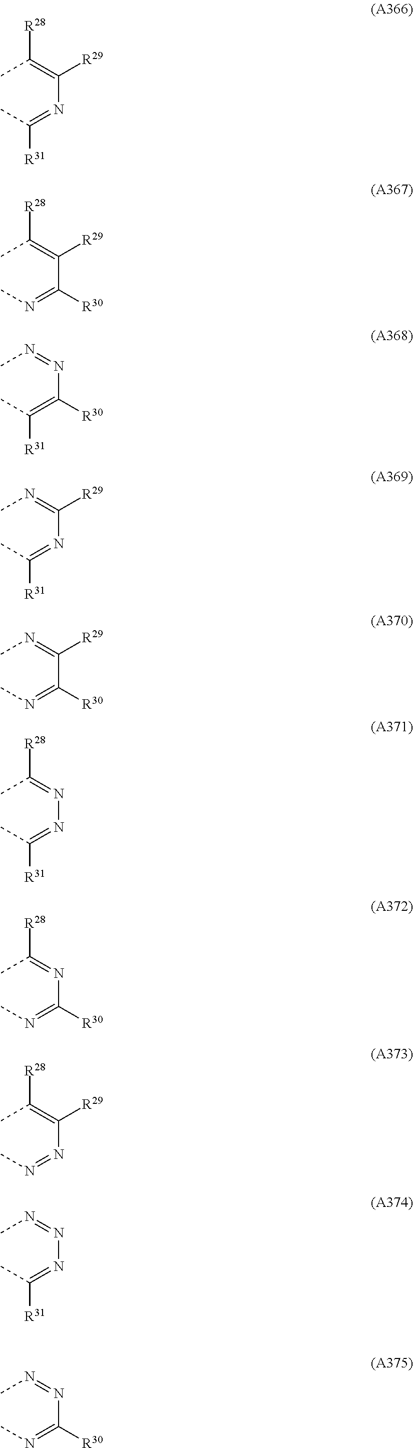

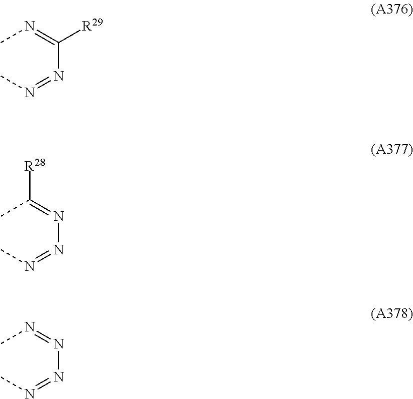

R⁷, R⁸, R¹², R¹³, R²² and R²³ are each independently hydrogen, halogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R²O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups,

R⁹, R¹⁰, R¹⁴, R¹⁵, R¹⁶, R¹⁷, R²⁹, R²¹, R²⁴ and R²⁵ are each independently hydrogen, halogen, (C₁-C₄)-alkyl, (C₁-C₄)-haloalkyl, (C₁-C₄)-alkoxy, (C₁-C₄)-haloalkoxy or (C₁-C₄)-alkoxy-(C₁-C₄)-alkyl or

any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or

any two geminal R⁹, R¹⁰, R¹⁴, R¹⁵, R¹⁶, R¹⁷, R²⁰, R²¹, R²⁴ and R²⁵ are an acetal of the formula —O—(C₂-C₄)-alkylene-O—,

R¹¹, R¹⁸, R¹⁹, R²⁶ ad R²⁷ are each independently hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of nitro, cyano, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,

or R¹¹, R¹⁸, R¹⁹, R²⁶ and R²⁷ are each independently (C₃-C₇)-cycloalkyl, heteroaryl, heterocyclyl or phenyl, where the four aforementioned radicals are each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, and where heterocyclyl bears n oxo groups,

R²⁸, R²⁹, R³⁰ and R³¹ are each independently hydrogen, nitro, halogen, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R²O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups,

and where the Q, R, X, W, R¹, R², R³, R⁴, R⁵, R⁶ and R′ radicals and the variables n and s are each as defined above.

Particular preference is given to compounds of the general formula (I) in which,

Q is a Q1, Q2, Q3 or Q4 radical,

X is nitro, halogen, cyano, (C₁-C₆)-alkyl, halo-(C₁-C₆)-alkyl, (C₂-C₆)-alkenyl, (C₂-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C, R¹(R¹ON═)C, R¹O(O)C, (R¹)₂N(O)C, R¹O, (R¹)₂N, R¹(O)C(R¹)N, R²(O)₂S(R¹)N, R²O(O)C(R¹)N, (R¹)₂N(O)C(R¹)N, R²(O)ₙS, R¹O(O)₂S, (R¹)₂N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, R¹O, (R¹)₂N, R²(O)ₙS, R¹O(O)₂S, (R¹)₂N(O)₂S and R¹O—(C₁-C₆)-alkyl, and where heterocyclyl bears n oxo groups,

W is hydrogen, halogen, nitro, cyano, (C₁-C₆)-alkyl, halo-(C₁-C₆)-alkyl, (C₃-C₇)-cycloalkyl, (C₁-C₆)-alkoxy, (C₁-C₆)-alkyl-(O)ₙS—, R¹O(O)C, (R¹)₂N, R¹(O)C(R¹)N or R²(O)₂S(R¹)N,

R is hydrogen,

R^{X} is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy and halogen, and where heterocyclyl bears n oxo groups,

or R^{X} is (C₃-C₇)-cycloalkyl, where this radical is substituted by s radicals from the group consisting of halogen, (C₁-C₆)-alkyl and halo-(C₁-C₆)-alkyl,

R^{Y} is hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,

R^{Z} is hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,

R¹ is hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine latter radicals are each substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl bears n oxo groups,

R² is (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine latter radicals are each substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl bears n oxo groups,

R³ is hydrogen or (C₁-C₆)-alkyl,

R⁴ is (C₁-C₆)-alkyl,

R⁵ is hydrogen or (C₁-C₄)-alkyl,

R′ is acetoxy, acetamido, methoxycarbonyl or (C₃-C₆)-cycloalkyl,

n is 0, 1 or 2,

s is 0, 1, 2 or 3.

L is a bridge selected from the group consisting of A1, A2, A3, A4, A5, A6, A7, A8, A9, A10, A11, A12, A13, A14, A17, A25, A26, A27, A28, A29, A30, A31, A32, A33, A34, A35, A36, A37, A38, A41, A49, A50, A51, A53, A55, A57, A59, A61, A62, A72, A139, A140, A141, A142, A143, A144, A145, A146, A147, A148, A149, A150, A151, A157, A158, A168, A274, A275, A276, A277, A278, A279, A280, A281, A282, A283, A284, A285, A286, A287, A363, A364, A365, A366, A367, A368, A369, A370, A371, A372 and A373,

R⁷, R⁸, R¹², R¹³, R²² and R²³ are each independently hydrogen, halogen, (C₁-C₆)-alkyl, halo-(C₁-C₆)-alkyl, (C₃-C₆)-cycloalkyl, halo-(C₃-C₆)-cycloalkyl, R¹(O)C, R¹(R¹ON═)C, R¹O(O)C, (R¹)₂N(O)C, R²O, R¹(O)CO, (R¹)₂N, R¹(O)C(R¹)N, R²(O)ₙS, R¹O—(C₁-C₆)-alkyl or R²(O)ₙS—(C₁-C₆)-alkyl,

R⁹, R¹⁰, R¹⁴, R¹⁵, R¹⁶, R¹⁷, R²⁰, R²¹, R²⁴ and R²⁵ are each independently hydrogen, halogen, (C₁-C₄)-alkyl, (C₁-C₄)-haloalkyl or (C₁-C₄)-alkoxy, or

any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or

any two geminal R⁹, R¹⁰, R¹⁴, R¹⁵, R¹⁶, R¹⁷, R²⁰, R²¹, R²⁴ and R²⁵ are an acetal of the formula —O—(C₂-C₄)-alkylene-O—,

R¹¹, R¹⁸, R¹⁹, R²⁶ and R²⁷ are each independently hydrogen or (C₁-C₆)-alkyl, where the (C₁-C₆)-alkyl group is substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,

or R¹¹, R¹⁸, R¹⁹, R²⁶ and R²⁷ are each independently (C₃-C₇)-cycloalkyl, heteroaryl, heterocyclyl or phenyl, where the four aforementioned radicals are each substituted by s radicals from the group consisting of halogen, nitro, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (Cᵣ C₆)-alkoxy-(C₁-C₄)-alkyl,

R²⁸, R²⁹, R³⁰ and R³¹ are each independently hydrogen, nitro, halogen, cyano, (C₁-C₆)-alkyl, halo-(C₁-C₆)-alkyl, (C₃-C₆)-cycloalkyl, halo-(C₃-C₆)-cycloalkyl, R¹(O)C, R¹(R¹ON═)C, R¹O(O)C, (R¹)₂N(O)C, R²O, R¹(O)CO, (R¹)₂N, R¹(O)C(R¹)N, R²(O)ₙS, R¹O(O)₂S, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)CO—(C₁-C₆)-alkyl, (R¹)₂N—(C₁-C₆)-alkyl, R¹(O)C(R¹)N—(C₁-C₆)-alkyl, R²(O)ₙS—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups.

Very particular preference is given to compounds of the general formula (I) in which

Q is a Q1, Q2, Q3 or Q4 radical

X is nitro, halogen, methyl, ethyl, n-propyl, isopropyl, trifluoromethyl, difluoromethyl, chlorodifluoromethyl, dichlorofluoromethyl, trichloromethyl, pentafluoroethyl, heptafluoroisopropyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl,

W is hydrogen, chlorine or methyl,

R is hydrogen,

R^{X} is methyl, ethyl, n-propyl, prop-2-en-1-yl, methoxyethyl, ethoxyethyl or methoxyethoxyethyl,

R^{Y} is methyl, ethyl, n-propyl, chlorine or amino,

R^{Z} is methyl, ethyl, n-propyl or methoxymethyl.

L is a bridge selected from the group consisting of A1, A2, A4, A5, A6, A7, A8, A25, A26, A28, A29, A30, A31, A32, A49, A50, A51, A53, A55, A57, A59, A61, A139, A140, A141, A142, A143, A145, A146, A147, A148, A149, A150, A274, A275, A278, A279, A280, A281, A282, A283, A284, A285, A286, A363, A364, A365, A366, A367, A368, A369, A370, A371, A372 and A373,

R⁷, R⁸, R¹², R¹³, R²² and R²³ are each independently hydrogen, halogen, methyl, ethyl, n-propyl, isopropyl, trifluoromethyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl,

R⁹, R¹⁰, R¹⁴, R¹⁵, R¹⁶, R¹⁷, R²⁰, R²¹, R²⁴ and R²⁵ are each independently hydrogen, halogen, methyl, methoxy, ethoxy or

any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or

any two geminal R⁹, R¹⁰, R¹⁴, R¹⁵, R¹⁶, R¹⁷, R²⁰, R²¹, R²⁴ and R²⁵ are an acetal of the formula —O—(CH₂)₂—O—,

R¹ is hydrogen, methyl or ethyl,

R¹¹, R¹⁹, R²⁶ and R²⁷ are each independently hydrogen or methyl,

R²⁸, R²⁹, R³⁰ and R³¹ are each independently hydrogen, nitro, halogen, methyl, ethyl, n-propyl, isopropyl, trifluoromethyl, difluoromethyl, chlorodifluoromethyl, dichlorofluoromethyl, trichloromethyl, pentafluoroethyl, heptafluoroisopropyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl.

In all the formulae specified hereinafter, the substituents and symbols have the same meaning as described in formula (I), unless defined differently.

Inventive compounds in which Q is Q1 or Q2 can be prepared, for example, by the method shown in scheme 1, by base-catalyzed reaction of a benzoyl chloride (II) with a 5-amino-1H-1,2,4-triazole or 5-amino-1H-tetrazole (III):

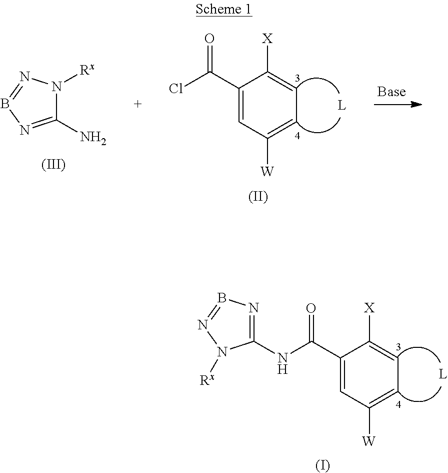

B therein is CH or N. The benzoyl chlorides of the formula (II) or their parent benzoic acids are known in principle and can be prepared, for example, by the methods described in DE 19532312 and WO 98/12192.

Inventive compounds in which Q is Q1 or Q2 can also be prepared by the method shown in scheme 2, by reaction of a benzoic acid of the formula (IV) with a 5-amino-1H-1,2,4-triazole or 5-amino-1H-tetrazole (III):

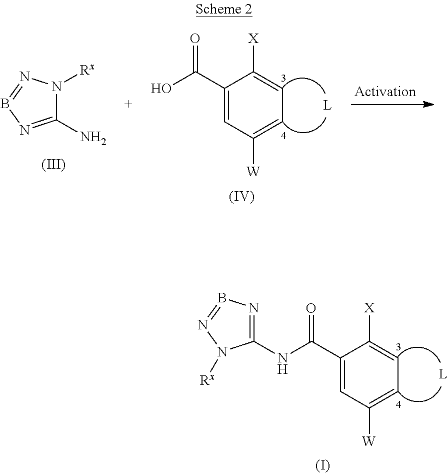

For the activation, it is possible to use dehydrating reagents which are typically used for amidation reactions, for example 1,1′-carbonyldiimidazole (CDI), dicyclohexylcarbodiimide (DCC), 2,4,6-tripropyl-1,3,5,2,4,6-trioxatriphosphinane 2,4,6-trioxide (T3P), etc.

Inventive compounds in which Q is Q1 or Q2 can also be prepared by the method shown in scheme 3, by conversion of an N-(1H-1,2,4-triazol-5-yl)benzamide or of an N-(1H-tetrazol-5-yl)benzamide:

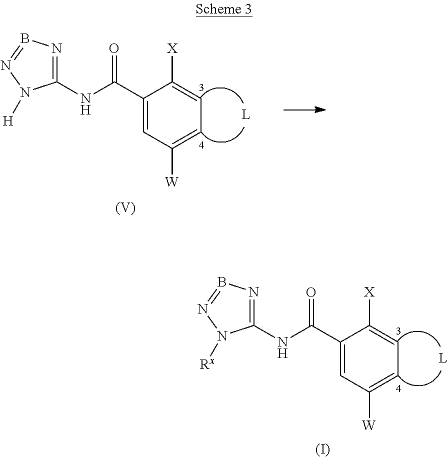

For this reaction shown above, it is possible, for example, to use alkylating agents, for example alkyl halides or sulfonates or dialkyl sulfates, in the presence of a base.

The 5-amino-1H-tetrazoles of the formula (III) are either commercially available or can be prepared analogously to methods known from the literature. For example, substituted 5-aminotetrazoles can be prepared from aminotetrazole by the method described in Journal of the American Chemical Society (1954), 76, 923-924:

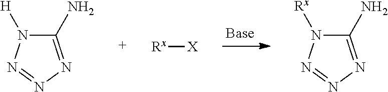

In the above reaction, X is a leaving group such as iodine. Substituted 5-aminotetrazoles can also be synthesized, for example, as described in Journal of the American Chemical Society (1954) 76, 88-89:

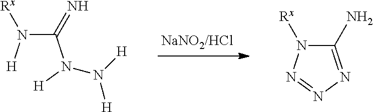

The 5-amino-1H-triazoles of the formula (III) are either commercially available or can be prepared analogously to methods known from the literature. For example, substituted 5-aminotriazoles can be prepared from aminotriazole by the method described in Zeitschrift für Chemie (1990), 30(12), 436-437:

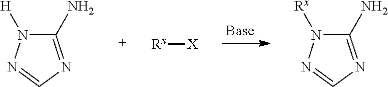

In the above reaction, X is a leaving group such as iodine. Substituted 5-aminotriazoles can also be synthesized, for example, as described in Chemische Berichte (1964), 97(2), 396-404:

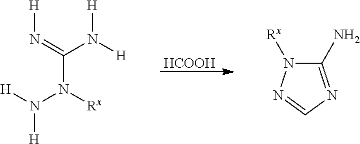

Substituted 5-aminotriazoles can also be synthesized, for example, as described in Angewandte Chemie (1963), 75, 918:

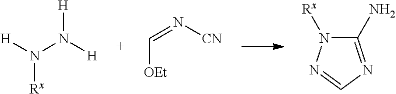

Inventive compounds in which Q is Q3 can be prepared, for example, by the method shown in scheme 4, by base-catalyzed reaction of a benzoyl chloride (II) with a 4-amino-1,2,5-oxadiazole (VI):

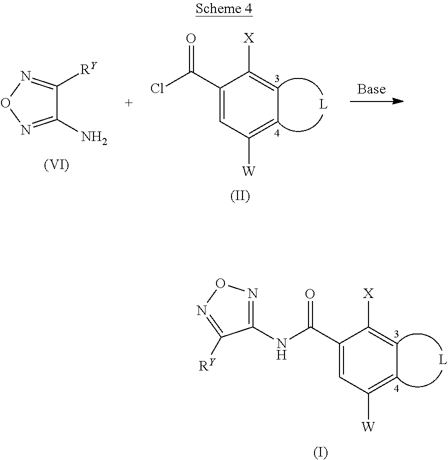

Inventive compounds can also be prepared by the method described in scheme 5, by reacting a benzoic acid of the formula (IV) with a 4-amino-1,2,5-oxadiazole (VI):

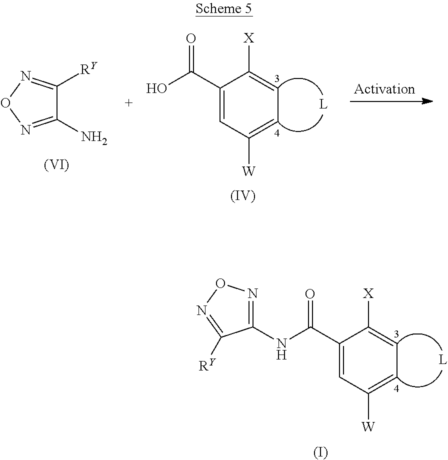

For the activation, it is possible to use dehydrating reagents which are typically used for amidation reactions, for example 1,1′-carbonyldiimidazole (CDI), dicyclohexylcarbodiimide (DCC), 2,4,6-tripropyl-1,3,5,2,4,6-trioxatriphosphinane 2,4,6-trioxide (T3P) etc.

The 4-amino-1,2,5-oxadiazoles of the formula (VI) are either commercially available or known, or can be prepared analogously to methods known from the literature.

For example, 3-alkyl-4-amino-1,2,5-oxadiazoles can be prepared from β-keto esters by the method described in Russian Chemical Bulletin, Int. Ed., vol. 54, 4, p. 1032-1037 (2005):

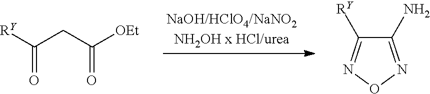

3-Aryl-4-amino-1,2,5-oxadiazoles can be synthesized, for example, as described in Russian Chemical Bulletin, 54(4), 1057-1059, (2005) or Indian Journal of Chemistry, Section B: Organic Chemistry Including Medicinal Chemistry, 26B(7), 690-2, (1987):

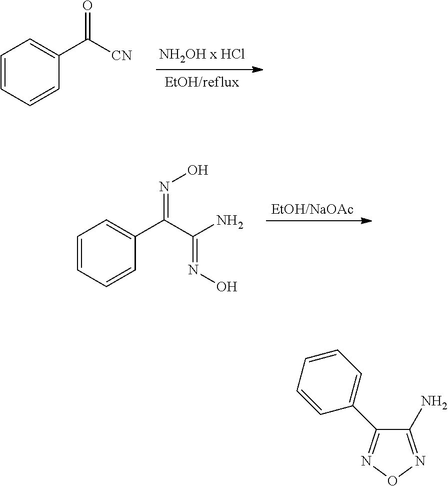 [SMILES: N#CC(=O)c1ccccc1 ; NC(=N\O)/C(=N\O)c1ccccc1 ; Nc1nonc1-c1ccccc1]

3-Amino-4-halo-1,2,5-oxadiazoles can be prepared, for example, by a Sandmeyer reaction from the commercially available 3,4-diamino-1,2,5-oxadiazole, according to the method described in Heteroatom Chemistry 15(3), 199-207 (2004):

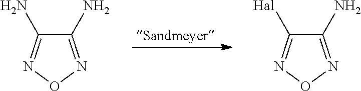

Nucleophilic R^{Y} radicals can be introduced into 3-amino-1,2,5-oxadiazoles by substitution of the leaving group L as described in Journal of Chemical Research, Synopses, (6), 190, 1985 or in or Izvestiya Akademii Nauk SSSR, Seriya Khimicheskaya, (9), 2086-8, 1986 or in Russian Chemical Bulletin (Translation of Izvestiya Akademii Nauk, Seriya Khimicheskaya), 53(3), 596-614, 2004. L is a leaving group, for example chlorine, bromine, iodine, mesyloxy, tosyloxy, trifluorosulfonyloxy, etc.

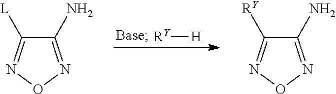

Inventive compounds in which Q is Q4 can be prepared, for example, by the method shown in scheme 6, by base-catalyzed reaction of a benzoyl chloride (II) with a 2-amino-1,3,4-oxadiazole (VII):

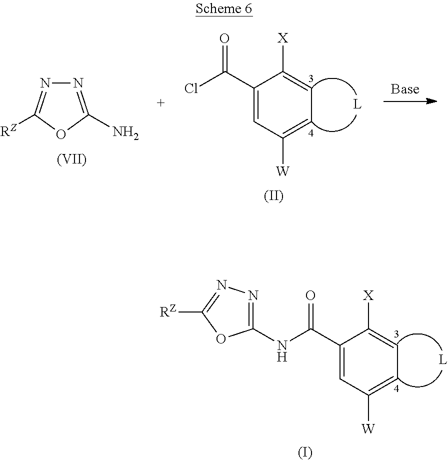

Inventive compounds can also be prepared by the method described in scheme 7, by reacting a benzoic acid of the formula (IV) with a 2-amino-1,3,4-oxadiazole (VII):

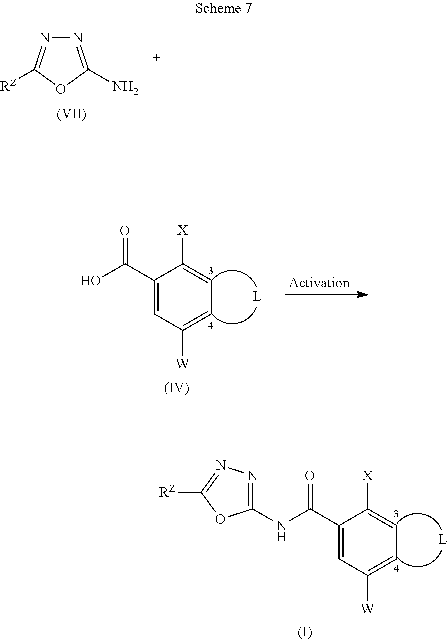

For the activation, it is possible to use dehydrating reagents which are typically used for amidation reactions, for example 1,1′-carbonyldiimidazole (CD), dicyclohexylcarbodiimide (DCC), 2,4,6-tripropyl-1,3,5,2,4,6-trioxatriphosphinane 2,4,6-trioxide (T3P), etc.

Inventive compounds can also be prepared by the method described in scheme 8, by cyclizing a compound of the formula VIII:

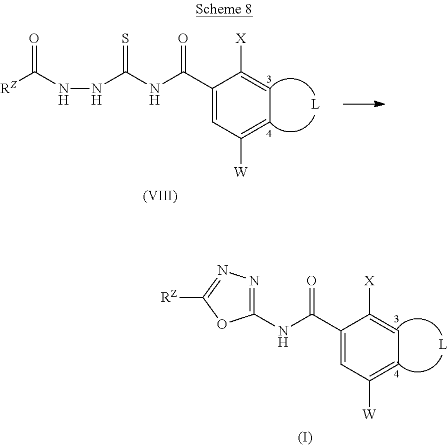

The cyclization can be performed by the methods described in Synth. Commun. 31 (12), 1907-1912 (2001) or in Indian J. Chem., Section B: Organic Chemistry Including Medicinal Chemistry; Vol. 43 (10), 2170-2174 (2004).

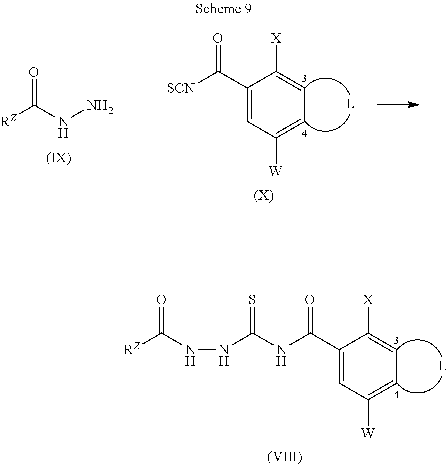

The compound of the formula VIII used in scheme 8 can be prepared by reaction of an acyl isothiocyanate of the formula X with a hydrazide of the formula IX by the method described in Synth. Commun. 25(12), 1885-1892 (1995).

Inventive compounds in which the substituent R is not hydrogen can be prepared, for example, according to the method shown in scheme 10, by reacting an N-(1,2,5-oxadiazol-3-yl)-, N-(1,3,4-oxadiazol-2-yl)-, N-(tetrazol-5-yl)- or N-(triazol-5-yl)bicycloarylcarboxamide (I) with a compound of the general formula (XI):

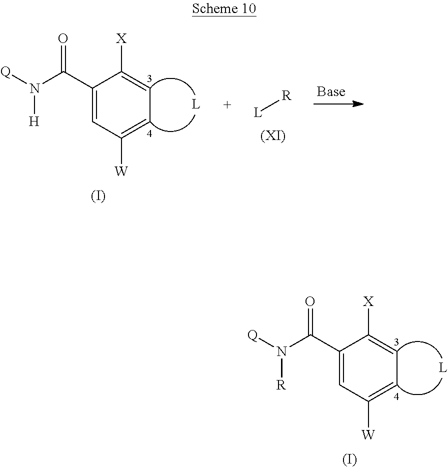

The compounds of the formula (XI) in which L is a leaving group, for example chlorine, bromine, iodine, methylsulfonyloxy, tosyloxy or trifluorosulfonyloxy are either commercially available or can be prepared by known methods described in the literature.

Inventive compounds can also be prepared according to the method shown in scheme 11 by reaction of an amine of the formula (XII) with an acid chloride (II), as described, for example, in J. Het. Chem. (1972), 9 (1), 107-109:

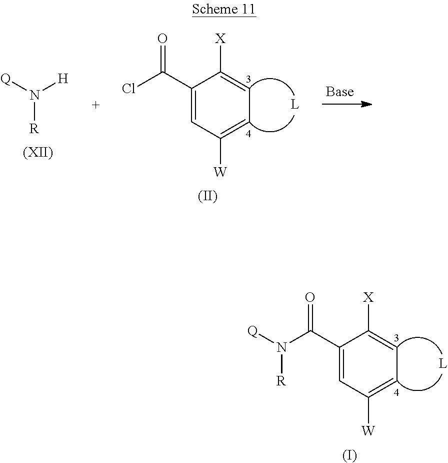

Inventive compounds can also be prepared according to the method shown in scheme 12, by reaction of an amine of the formula (XII) with an acid of the formula (IV):

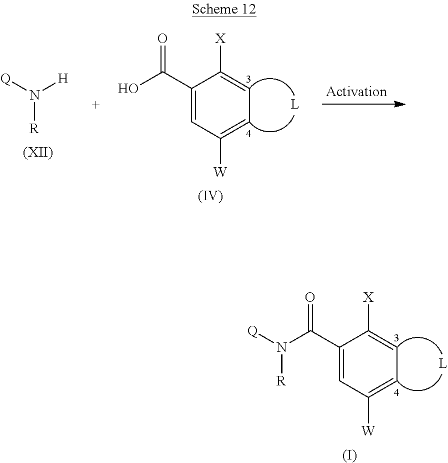

For the activation, it is possible to use dehydrating reagents which are typically used for amidation reactions, for example 1,1′-carbonyldiimidazole (CD), dicyclohexylcarbodiimide (DCC), 2,4,6-tripropyl-1,3,5,2,4,6-trioxatriphosphinane 2,4,6-trioxide (T3P), etc.

The amines of the formula (XII) are either commercially available or known in the literature or can be prepared, for example, by the method described in scheme 13, by base-catalyzed alkylation or by reductive alkylation, or according to the method described in scheme 14, by nucleophilic substitution of a leaving group L, for example chlorine, by amines R—NH₂.

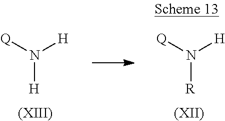

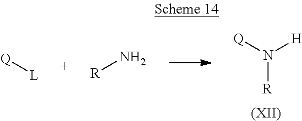

The amines of the formula (XII) can also be prepared by cyclization reactions as described, for example, in J. Org. Chem. 73(10), 3738-3744 (2008) where Q=Q1, or in Buletinul Institutului Politehnic din Iasi (1974), 20(1-2), 95-99 or in J. Org. Chem. 67(21), 7361-7364 (2002) where Q=Q4.

It may be expedient to change the order of the reaction steps. For instance, benzoic acids bearing a sulfoxide cannot be converted directly to their acid chlorides. Here, it is advisable to prepare initially, at the thioether stage, the amide and then to oxidize the thioether to the sulfoxide.

The workup of the respective reaction mixtures is generally effected by known processes, for example by crystallization, aqueous-extractive workup, by chromatographic methods or by a combination of these methods.

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the work-up or the purification of the products and/or intermediates. Overall, this is understood to mean a procedure as described, for example, by D. Tiebes in Combinatorial Chemistry—Synthesis, Analysis, Screening (editor Günther Jung), Wiley, 1999, on pages 1 to 34.

For the parallelized conduct of the reaction and workup, it is possible to use a number of commercially available instruments, for example Calypso reaction blocks from Barnstead International, Dubuque, Iowa 52004-0797, USA or reaction stations from Radleys, Shirehill, Saffron Walden, Essex, CB11 3AZ, England, or MultiPROBE Automated Workstations from PerkinElmer, Waltham, Mass. 02451, USA. For the parallelized purification of compounds of the general formula (I) and salts thereof or of intermediates which occur in the course of preparation, available apparatuses include chromatography apparatuses, for example from ISCO, Inc., 4700 Superior Street, Lincoln, Nebr. 68504, USA.

The apparatuses detailed lead to a modular procedure in which the individual working steps are automated, but manual operations have to be carried out between the working steps. This can be circumvented by using partly or fully integrated automation systems in which the respective automation modules are operated, for example, by robots. Automation systems of this type can be obtained, for example, from Caliper, Hopkinton, Mass. 01748, USA.

The implementation of single or multiple synthesis steps can be supported by the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Aside from the methods described here, the compounds of the general formula (I) and salts thereof can be prepared completely or partially by solid-phase-supported methods. For this purpose, individual intermediates or all intermediates in the synthesis or a synthesis adapted for the corresponding procedure are bound to a synthesis resin. Solid-phase-supported synthesis methods are described adequately in the technical literature, for example Barry A. Bunin in “The Combinatorial Index”, Academic Press, 1998 and Combinatorial Chemistry—Synthesis, Analysis, Screening (editor: Günther Jung), Wiley, 1999. The use of solid-phase-supported synthesis methods permits a number of protocols, which are known from the literature and which for their part may be performed manually or in an automated manner. The reactions can be performed, for example, by means of IRORI technology in microreactors from Nexus Biosystems, 12140 Community Road, Poway, Calif. 92064, USA.

Both in the solid and in the liquid phase, individual or several synthesis steps may be supported by the use of microwave technology. The specialist literature describes a series of experimental protocols, for example in Microwaves in Organic and Medicinal Chemistry (editor: C. O. Kappe and A. Stadler), Wiley, 2005.

The preparation by the processes described here gives compounds of the formula (I) and salts thereof in the form of substance collections, which are called libraries. The present invention also provides libraries comprising at least two compounds of the formula (I) and salts thereof.

The inventive compounds of the formula (I) (and/or salts thereof), referred to collectively as “inventive compounds” hereinafter, have excellent herbicidal efficacy against a broad spectrum of economically important monocotyledonous and dicotyledonous annual harmful plants. The active ingredients also have good control over perennial weed plants which are difficult to control and produce shoots from rhizomes, root stocks or other perennial organs.

The present invention therefore also provides a method for controlling unwanted plants or for regulating the growth of plants, preferably in plant crops, in which one or more inventive compound(s) is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The inventive compounds can be deployed, for example, prior to sowing (if appropriate also by incorporation into the soil), prior to emergence or after emergence. Specific examples of some representatives of the monocotyledonous and dicotyledonous weed flora which can be controlled by the inventive compounds are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the inventive compounds are applied to the soil surface before germination, either the emergence of the weed seedlings is prevented completely or the weeds grow until they have reached the cotyledon stage, but then they stop growing and ultimately die completely after three to four weeks have passed.

If the active ingredients are applied post-emergence to the green parts of the plants, growth stops after the treatment, and the harmful plants remain at the growth stage of the time of application, or they die completely after a certain time, such that competition by the weeds, which is harmful to the crop plants, is thus eliminated very early and in a lasting manner.

Although the inventive compounds have outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, will be damaged to a negligible extent only, if at all, depending on the structure of the particular inventive compound and its application rate. For these reasons, the present compounds are very suitable for selective control of unwanted plant growth in plant crops such as agriculturally useful plants or ornamental plants.

In addition, the inventive compounds (depending on their particular structure and the application rate deployed) have outstanding growth-regulating properties in crop plants. They intervene in the plants' own metabolism with regulatory effect, and can thus be used for controlled influencing of plant constituents and to facilitate harvesting, for example by triggering desiccation and stunted growth. In addition, they are also suitable for general control and inhibition of unwanted vegetative growth without killing the plants. Inhibition of vegetative growth plays a major role for many mono- and dicotyledonous plants since, for example, this can reduce or completely prevent lodging.

By virtue of their herbicidal and plant-growth-regulating properties, the active ingredients can also be used for controlling harmful plants in crops of genetically modified plants or plants modified by conventional mutagenesis. In general, transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other particular properties relate, for example, to the harvested material with regard to quantity, quality, storability, composition and specific constituents. For instance, there are known transgenic plants with an elevated starch content or altered starch quality, or those with a different fatty acid composition in the harvested material.

With regard to transgenic crops, preference is given to the use of the inventive compounds in economically important transgenic crops of useful plants and ornamentals, for example of cereals such as wheat, barley, rye, oats, millet/sorghum, rice and corn, or else crops of sugar beet, cotton, soybean, oilseed rape, potato, tomato, peas and other vegetables. Preferably, the inventive compounds can be used as herbicides in crops of useful plants which are resistant, or have been made resistant by genetic engineering, to the phytotoxic effects of the herbicides.

Preference is given to the use of the inventive compounds or salts thereof in economically important transgenic crops of useful plants and ornamentals, for example of cereals such as wheat, barley, rye, oats, millet/sorghum, rice, cassava and corn, or else crops of sugar beet, cotton, soybean, oilseed rape, potato, tomato, peas and other vegetables. Preferably, the inventive compounds can be used as herbicides in crops of useful plants which are resistant, or have been made resistant by genetic engineering, to the phytotoxic effects of the herbicides.

Conventional ways of producing novel plants which have modified properties in comparison to plants which have occurred to date consist, for example, in traditional breeding methods and the generation of mutants. Alternatively, novel plants with modified properties can be generated with the aid of recombinant methods (see, for example, EP-A-0221044, EP-A-0131624). For example, there have been many descriptions of
recombinant modifications of crop plants for the purpose of modifying the starch synthesized in the plants (for example WO 92/11376, WO 92/14827,

WO 91/19806),
transgenic crop plants which are resistant to particular herbicides of the glufosinate type (cf., for example, EP-A-0242236, EP-A-242246) or glyphosate type
(WO 92/00377) or of the sulfonylureas (EP-A-0257993, U.S. Pat. No. 5,013,659),
transgenic crop plants, for example cotton, with the ability to produce Bacillus thuringiensis toxins (Bt toxins), which make the plants resistant to particular pests (EP-A-0142924, EP-A-0193259),
transgenic crop plants with a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants with reduced photorespiration which feature higher yields and higher stress tolerance (EPA 0305398),
transgenic crop plants which produce pharmaceutically or diagnostically important proteins (“molecular pharming”),
transgenic crop plants which feature higher yields or better quality,
transgenic crop plants which feature a combination, for example, of the abovementioned novel properties (“gene stacking”).

Numerous molecular biology techniques which can be used to produce novel transgenic plants with modified properties are known in principle; see, for example, I. Potrykus and G. Spangenberg (eds.), Gene Transfer to Plants, Springer Lab Manual (1995), Springer Verlag Berlin, Heidelberg or Christou, “Trends in Plant Science” 1 (1996) 423-431.

For such recombinant manipulations, nucleic acid molecules which allow mutagenesis or sequence alteration by recombination of DNA sequences can be introduced into plasmids. With the aid of standard methods, it is possible, for example, to undertake base exchanges, remove part-sequences or add natural or synthetic sequences. For the joining of the DNA fragments to one another, adaptors or linkers can be attached to the fragments; see, for example, Sambrook et al., 1989, Molecular Cloning, A Laboratory Manual, 2nd ed. Cold Spring Harbor Laboratory Press, Cold Spring Harbor, N.Y.; or Winnacker “Gene and Klone” [Genes and Clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to link the coding region with DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

Preferably, the inventive compounds can be used in transgenic crops which are resistant to growth regulators, for example dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients.

When the inventive active ingredients are used in transgenic crops, not only do the effects toward harmful plants which are observed in other crops occur, but often also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also provides for the use of the inventive compounds as herbicides for control of harmful plants in transgenic crop plants.

The inventive compounds can be applied in the form of wettable powders, emulsifiable concentrates, sprayable solutions, dusting products or granules in the customary formulations. The invention therefore also provides herbicidal and plant-growth-regulating compositions which comprise the inventive compounds.

The inventive compounds can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusting products (DP), seed-dressing products, granules for scattering and soil application, granules (GR) in the form of microgranules, spray granules, coated granules and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [Chemical Technology], volume 7, C. Hanser Verlag Munich, 4th edition 1986, Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation assistants, such as inert materials, surfactants, solvents and further additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesell., Stuttgart 1976, Winnacker-Küchler, “Chemische Technologie”, Volume 7, C. Hanser Verlag Munich, 4th ed. 1986.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix. Suitable safeners are, for example, mefenpyr-diethyl, cyprosulfamide, isoxadifen-ethyl, cloquintocet-mexyl and dichlormid.

Wettable powders are preparations which can be dispersed uniformly in water and, in addition to the active ingredient, apart from a diluent or inert substance, also comprise surfactants of the ionic and/or nonionic type (wetting agents, dispersants), for example polyethoxylated alkylphenols, polyethoxylated fatty alcohols, polyethoxylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatus such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dustable powders are obtained by grinding the active ingredient with finely distributed solid substances, for example talc, natural clays such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be prepared either by spraying the active ingredient onto adsorptive granular inert material or by applying active ingredient concentrates to the surface of carriers, such as sand, kaolinites or granular inert material, by means of adhesives, for example polyvinyl alcohol, sodium polyacrylates or else mineral oils. Suitable active ingredients can also be granulated in the manner customary for the production of fertilizer granules—if desired as a mixture with fertilizers.

Water-dispersible granules are produced generally by the customary processes such as spray-drying, fluidized bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of inventive compounds.

In wettable powders, the active ingredient concentration is, for example, about 10 to 90% by weight, the remainder to 100% by weight consisting of customary formulation constituents. In emulsifiable concentrates, the active ingredient concentration may be about 1 to 90% and preferably 5 to 80% by weight. Dust-type formulations contain 1 to 30% by weight of active ingredient, preferably usually 5 to 20% by weight of active ingredient; sprayable solutions contain about 0.05 to 80% and preferably 2 to 50% by weight of active ingredient. In the case of water-dispersible granules, the active ingredient content depends partially on whether the active compound is present in liquid or solid form and on which granulation auxiliaries, fillers, etc., are used. In the water-dispersible granules, the content of active ingredient is, for example, between 1 and 95% by weight, preferably between 10 and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix.

For application, the formulations in commercial form are, if appropriate, diluted in a customary manner, for example in the case of wettable powders, emulsifiable concentrates, dispersions and water-dispersible granules with water. Dust-type formulations, granules for soil application or granules for scattering and sprayable solutions are not normally diluted further with other inert substances prior to application.

The required application rate of the compounds of the formula (I) varies with the external conditions, including temperature, humidity and the type of herbicide used. It can vary within wide limits, for example between 0.001 and 1.0 kg/ha or more of active substance, but it is preferably between 0.005 and 750 g/ha.

The examples below illustrate the invention.

### A. CHEMICAL EXAMPLES

### Synthesis of 7-methyl-N-(1-methyl-1H-tetrazol-5-yl)-1-benzothiophene-6-carboxamide

### Example No. 1-1

### Step 1: Synthesis of methyl 7-methyl-1-benzylthiophene-6-carboxylate

The synthesis of methyl 3-hydroxy-7-methyl-2,3-dihydro-1-benzothiophene-6-carboxylate is known and is described, for example, in DE 19532312 (example 16). 619 mg (3 mmol) of para-toluenesulfonic acid were added to a solution of 7.3 g (32 mmol) of methyl 3-hydroxy-7-methyl-2,3-dihydro-1-benzothiophene-6-carboxylate in 92 ml of toluene. The mixture was heated under reflux for 1 h. Subsequently, the mixture was cooled to room temperature (RT) and washed with 60 ml of a saturated aqueous sodium hydrogencarbonate solution. After phase separation, the organic phase was dried and the filtrate was freed from the solvent. 6.0 g of the desired product were obtained.

### Step 2: Synthesis of 7-methyl-1-benzothiophene-6-carboxylic acid

A solution of 6.0 g (28 mmol) of methyl 7-methyl-1-benzothiophene-6-carboxylate in a mixture of 50 ml of methanol and 10 ml of water was admixed with 1.73 g (43 mmol) of sodium hydroxide. The reaction mixture was heated under reflux for 1 h. Then the methanol was substantially removed and the residue was taken up in 10 ml of water. The mixture was washed twice with 10 ml each time of diethyl ether. Thereafter, the aqueous phase was acidified with 2M hydrochloric acid. The resultant suspension was filtered and the isolated solids were washed with 20 ml of water and finally dried. 4.1 g of the desired product were obtained.

### Step 3: Synthesis of 7-methyl-N-(1-methyl-1H-tetrazol-5-yl)-1-benzothiophene-6-carboxamide

500 mg (2.60 mmol) of 7-methyl-1-benzothiophene-6-carboxylic acid and 335 mg (98% purity; 3.31 mmol) of 5-amino-1-methyl-1H-tetrazole in 10 ml of dry pyridine were cooled to a temperature of 0° C.-5° C. Then 462 mg (3.64 mmol) of oxalyl chloride were added. The mixture was thawed to RT and then stirred at RT for another 2 h. Then a further 115.5 mg (0.91 mmol) of oxalyl chloride were added at RT. The reaction mixture was stirred at RT for 16 h. For workup, the solvent was removed. The residue was taken up in dichloromethane and and the mixture was washed with saturated aqueous sodium hydrogencarbonate solution. After phase separation, the organic phase was freed of the solvent. The residue was stirred with acetonitrile and filtered. The residue obtained was 355 mg of clean product.

The examples listed in the tables below were prepared analogously to the abovementioned methods or are obtainable analogously to the abovementioned methods. The compounds listed in the tables below are very particularly preferred.

The abbreviations used correspond to those that are commonly known and mean:

| | | | |
|---|---|---|---|
| Et = ethyl | Me = methyl | n-Pr = n-propyl | c-Pr = c-propyl |
| c = cyclo | t = tertiary | t-Bu = t-butyl | Ph = phenyl |

TABLE 1
Inventive compounds of the general formula (I) in which Q is Q1, R^{x} is a
methyl group and R is hydrogen, and L is the bridge A30 in which the R⁷
and R⁸ radicals are both hydrogen

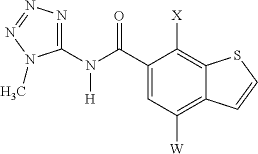

| No. | X | W | Physical data (¹H NMR) |
|---|---|---|---|
| 1-1 | Me | H | (400 MHz, DMSO-d₆ δ, ppm) 7.98 |
| | | | (d, 1H), 7.89 (d, 1H), 7.70 (d, 1H), 7.59 |
| | | | (d, 1H), 4.00 (s, 3H), 2.69 (s, 3H) |
| 1-2 | Et | H | |
| 1-3 | Cl | H | |
| 1-4 | OMe | H | |
| 1-5 | CF₃ | H | |
| 1-6 | SO₂Me | H | |
| 1-7 | CH₂OMe | H | |
| 1-8 | CH₂O(CH₂)₂OMe | H | |
| 1-9 | Me | Me | |
| 1-10 | Et | Me | |
| 1-11 | Cl | Me | |
| 1-12 | OMe | Me | |
| 1-13 | CF₃ | Me | |
| 1-14 | SO₂Me | Me | |
| 1-15 | CH₂OMe | Me | |
| 1-16 | CH₂O(CH₂)₂OMe | Me | |

TABLE 2
Inventive compounds of the general formula (I) in which Q is Q1, R^{x} is an
ethyl group and R is hydrogen, and L is the bridge A30 in which the R⁷
and R⁸ radicals are both hydrogen

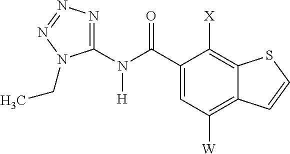

| No. | X | W | Physical data (¹H NMR) |
|---|---|---|---|
| 2-1 | Me | H | |
| 2-2 | Et | H | |
| 2-3 | Cl | H | |
| 2-4 | OMe | H | |
| 2-5 | CF₃ | H | |
| 2-6 | SO₂Me | H | |
| 2-7 | CH₂OMe | H | |
| 2-8 | CH₂O(CH₂)₂OMe | H | |
| 2-9 | Me | Me | |
| 2-10 | Et | Me | |
| 2-11 | Cl | Me | |
| 2-12 | OMe | Me | |
| 2-13 | CF₃ | Me | |
| 2-14 | SO₂Me | Me | |
| 2-15 | CH₂OMe | Me | |
| 2-16 | CH₂O(CH₂)₂OMe | Me | |

TABLE 3
Inventive compounds of the general formula (I) in which Q is Q1, R^{x} is an
n-propyl group and R is hydrogen, and L is the bridge A30 in which the
R⁷ and R⁸ radicals are both hydrogen

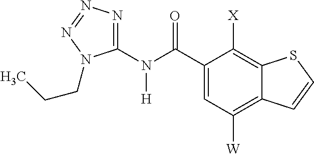

| No. | X | W | Physical data (¹H NMR) |
|---|---|---|---|
| 3-1 | Me | H | |
| 3-2 | Et | H | |
| 3-3 | Cl | H | |
| 3-4 | OMe | H | |
| 3-5 | CF₃ | H | |
| 3-6 | SO₂Me | H | |
| 3-7 | CH₂OMe | H | |
| 3-8 | CH₂O(CH₂)₂OMe | H | |
| 3-9 | Me | Me | |
| 3-10 | Et | Me | |
| 3-11 | Cl | Me | |
| 3-12 | OMe | Me | |
| 3-13 | CF₃ | Me | |
| 3-14 | SO₂Me | Me | |
| 3-15 | CH₂OMe | Me | |
| 3-16 | CH₂O(CH₂)₂OMe | Me | |

TABLE 4
Inventive compounds of the general formula (I) in which Q is Q2, R^{x} is a
methyl group and R is hydrogen, and L is the bridge A30 in which the R⁷
and R⁸ radicals are both hydrogen

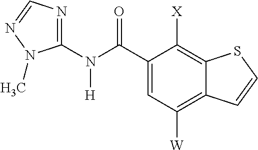

| No. | X | W | Physical data (¹H NMR) |
|---|---|---|---|
| 4-1 | Me | H | |
| 4-2 | Et | H | |
| 4-3 | Cl | H | |
| 4-4 | OMe | H | |
| 4-5 | CF₃ | H | |
| 4-6 | SO₂Me | H | |
| 4-7 | CH₂OMe | H | |
| 4-8 | CH₂O(CH₂)₂OMe | H | |
| 4-9 | Me | Me | |
| 4-10 | Et | Me | |
| 4-11 | Cl | Me | |
| 4-12 | OMe | Me | |
| 4-13 | CF₃ | Me | |
| 4-14 | SO₂Me | Me | |
| 4-15 | CH₂OMe | Me | |
| 4-16 | CH₂O(CH₂)₂OMe | Me | |

TABLE 5
Inventive compounds of the general formula (I) in which Q is Q3, R^{Y} is a
methyl group and R is hydrogen, and L is the bridge A30 in which the R⁷
and R⁸ radicals are both hydrogen

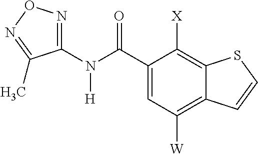

| No. | X | W | Physical data (¹H NMR) |
|---|---|---|---|
| 5-1 | Me | H | (400 MHz, CDCl₃ δ, ppm) 7.78 |
| | | | (d, 1H), 7.65 (d, 1H), 7.61 (d, 1H), 7.43 |
| | | | (d, 1H), 2.80 (s, 3H), 2.52 (s, 3H) |
| 5-2 | Et | H | |
| 5-3 | Cl | H | |
| 5-4 | OMe | H | |
| 5-5 | CF₃ | H | |
| 5-6 | SO₂Me | H | |
| 5-7 | CH₂OMe | H | |
| 5-8 | CH₂O(CH₂)₂OMe | H | |
| 5-9 | Me | Me | |
| 5-10 | Et | Me | |
| 5-11 | Cl | Me | |
| 5-12 | OMe | Me | |
| 5-13 | CF₃ | Me | |
| 5-14 | SO₂Me | Me | |
| 5-15 | CH₂OMe | Me | |
| 5-16 | CH₂O(CH₂)₂OMe | Me | |

TABLE 6
Inventive compounds of the general formula (I) in which Q is Q4, R^{Z} is a
methyl group and R is hydrogen, and L is the bridge A30 in which the R⁷
and R⁸ radicals are both hydrogen

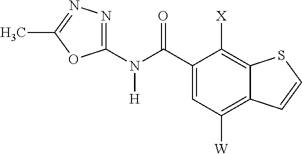

| No. | X | W | Physical data (¹H NMR) |
|---|---|---|---|
| 6-1 | Me | H | |
| 6-2 | Et | H | |
| 6-3 | Cl | H | |
| 6-4 | OMe | H | |
| 6-5 | CF₃ | H | |
| 6-6 | SO₂Me | H | |
| 6-7 | CH₂OMe | H | |
| 6-8 | CH₂O(CH₂)₂OMe | H | |
| 6-9 | Me | Me | |
| 6-10 | Et | Me | |
| 6-11 | Cl | Me | |
| 6-12 | OMe | Me | |
| 6-13 | CF₃ | Me | |
| 6-14 | SO₂Me | Me | |
| 6-15 | CH₂OMe | Me | |
| 6-16 | CH₂O(CH₂)₂OMe | Me | |

TABLE 7
Inventive compounds of the general formula (I) in which Q is Q1, R^{X} is a
methyl group and R is hydrogen, and L is the bridge A32 in which the R⁷
and R⁸ radicals are both hydrogen

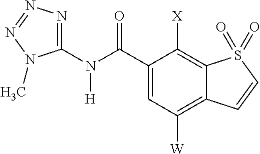

| No. | X | W | Physical data (¹H NMR) |
|---|---|---|---|
| 7-1 | Me | H | (400 MHz, DMSO-d₆ δ, ppm) 7.94 |
| | | | (d, 1H), 7.68 (d, 1H), 7.57 (d, 1H), 7.50 |
| | | | (d, 1H), 3.99 (s, 3H), 2.63 (s, 3H) |
| 7-2 | Et | H | |
| 7-3 | Cl | H | |
| 7-4 | OMe | H | |
| 7-5 | CF₃ | H | |
| 7-6 | SO₂Me | H | |
| 7-7 | CH₂OMe | H | |
| 7-8 | CH₂O(CH₂)₂OMe | H | |
| 7-9 | Me | Me | |
| 7-10 | Et | Me | |
| 7-11 | Cl | Me | |
| 7-12 | OMe | Me | |
| 7-13 | CF₃ | Me | |
| 7-14 | SO₂Me | Me | |
| 7-15 | CH₂OMe | Me | |
| 7-16 | CH₂O(CH₂)₂OMe | Me | |

TABLE 8
Inventive compounds of the general formula (I) in which Q is Q1, R^{X} is an
ethyl group and R is hydrogen, and L is the bridge A32 in which the R⁷
and R⁸ radicals are both hydrogen

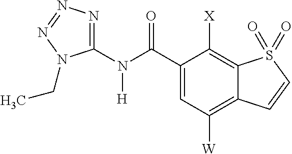

| No. | X | W | Physical data (¹H NMR) |
|---|---|---|---|
| 8-1 | Me | H | |
| 8-2 | Et | H | |
| 8-3 | Cl | H | |
| 8-4 | OMe | H | |
| 8-5 | CF₃ | H | |
| 8-6 | SO₂Me | H | |
| 8-7 | CH₂OMe | H | |
| 8-8 | CH₂O(CH₂)₂OMe | H | |
| 8-9 | Me | Me | |
| 8-10 | Et | Me | |
| 8-11 | Cl | Me | |
| 8-12 | OMe | Me | |
| 8-13 | CF₃ | Me | |
| 8-14 | SO₂Me | Me | |
| 8-15 | CH₂OMe | Me | |
| 8-16 | CH₂O(CH₂)₂OMe | Me | |

TABLE 9
Inventive compounds of the general formula (I) in which Q is Q1, R^{X} is an
n-propyl group and R is hydrogen, and L is the bridge A32 in which the
R⁷ and R⁸ radicals are both hydrogen

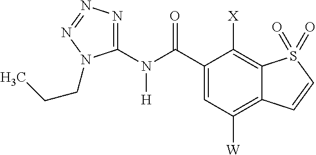

| No. | X | W | Physical data (¹H NMR) |
|---|---|---|---|
| 9-1 | Me | H | |
| 9-2 | Et | H | |
| 9-3 | Cl | H | |
| 9-4 | OMe | H | |
| 9-5 | CF₃ | H | |
| 9-6 | SO₂Me | H | |
| 9-7 | CH₂OMe | H | |
| 9-8 | CH₂O(CH₂)₂OMe | H | |
| 9-9 | Me | Me | |
| 9-10 | Et | Me | |
| 9-11 | Cl | Me | |
| 9-12 | OMe | Me | |
| 9-13 | CF₃ | Me | |
| 9-14 | SO₂Me | Me | |
| 9-15 | CH₂OMe | Me | |
| 9-16 | CH₂O(CH₂)₂OMe | Me | |

TABLE 10
Inventive compounds of the general formula (I) in which Q is Q2, R^{X} is a
methyl group and R is hydrogen, and L is the bridge A32 in which the R⁷
and R⁸ radicals are both hydrogen

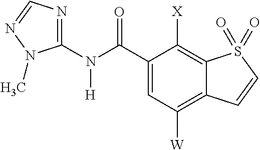

| No. | X | W | Physical data (¹H NMR) |
|---|---|---|---|
| 10-1 | Me | H | |
| 10-2 | Et | H | |
| 10-3 | Cl | H | |
| 10-4 | OMe | H | |
| 10-5 | CF₃ | H | |
| 10-6 | SO₂Me | H | |
| 10-7 | CH₂OMe | H | |
| 10-8 | CH₂O(CH₂)₂OMe | H | |
| 10-9 | Me | Me | |
| 10-10 | Et | Me | |
| 10-11 | Cl | Me | |
| 10-12 | OMe | Me | |
| 10-13 | CF₃ | Me | |
| 10-14 | SO₂Me | Me | |
| 10-15 | CH₂OMe | Me | |
| 10-16 | CH₂O(CH₂)₂OMe | Me | |

TABLE 11
Inventive compounds of the general formula (I) in which Q is Q3, R^{Y} is a
methyl group and R is hydrogen, and L is the bridge A32 in which the R⁷
and R⁸ radicals are both hydrogen

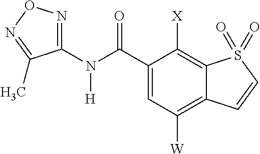

| No. | X | W | Physical data (¹H NMR) |
|---|---|---|---|
| 11-1 | Me | H | (400 MHz, CDCl₃ δ, ppm) 7.75 |
| | | | (d, 1H), 7.31 (d, 1H), 7.23 (d, 1H), |
| | | | 6.83 (d, 1H), 2.81 (s, 3H), 2.50 (s, 3H) |
| 11-2 | Et | H | |
| 11-3 | Cl | H | |
| 11-4 | OMe | H | |
| 11-5 | CF₃ | H | |
| 11-6 | SO₂Me | H | |
| 11-7 | CH₂OMe | H | |
| 11-8 | CH₂O(CH₂)₂OMe | H | |
| 11-9 | Me | Me | |
| 11-10 | Et | Me | |
| 11-11 | Cl | Me | |
| 11-12 | OMe | Me | |
| 11-13 | CF₃ | Me | |
| 11-14 | SO₂Me | Me | |
| 11-15 | CH₂OMe | Me | |
| 11-16 | CH₂O(CH₂)₂OMe | Me | |

TABLE 12
Inventive compounds of the general formula (I) in which Q is Q4, R^{Z} is a
methyl group and R is hydrogen, and L is the bridge A32 in which the R⁷
and R⁸ radicals are both hydrogen

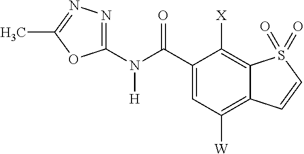

| No. | X | W | Physical data (¹H NMR) |
|---|---|---|---|
| 12-1 | Me | H | |
| 12-2 | Et | H | |
| 12-3 | Cl | H | |
| 12-4 | OMe | H | |
| 12-5 | CF₃ | H | |
| 12-6 | SO₂Me | H | |
| 12-7 | CH₂OMe | H | |
| 12-8 | CH₂O(CH₂)₂OMe | H | |
| 12-9 | Me | Me | |
| 12-10 | Et | Me | |
| 12-11 | Cl | Me | |
| 12-12 | OMe | Me | |
| 12-13 | CF₃ | Me | |
| 12-14 | SO₂Me | Me | |
| 12-15 | CH₂OMe | Me | |
| 12-16 | CH₂O(CH₂)₂OMe | Me | |

TABLE 13
Inventive compounds of the general formula (I) in which Q is Q1,
R^{X} is a methyl group and R is hydrogen, and L is the bridge
A8 in which the R⁷ radical is hydrogen

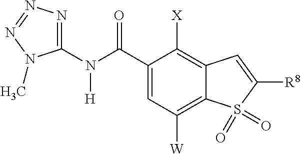

| No. | X | W | R⁸ | Physical data (¹H NMR) |
|---|---|---|---|---|
| 13-1 | Me | H | H | |
| 13-2 | Et | H | H | |
| 13-3 | Cl | H | H | |
| 13-4 | OMe | H | H | |
| 13-5 | CF₃ | H | H | |
| 13-6 | SO₂Me | H | H | |
| 13-7 | CH₂OMe | H | H | |
| 13-8 | CH₂O(CH₂)₂OMe | H | H | |
| 13-9 | Me | Me | H | |
| 13-10 | Et | Me | H | |
| 13-11 | Cl | Me | H | |
| 13-12 | OMe | Me | H | |
| 13-13 | CF₃ | Me | H | |
| 13-14 | SO₂Me | Me | H | |
| 13-15 | CH₂OMe | Me | H | |
| 13-16 | CH₂O(CH₂)₂OMe | Me | H | |
| 13-17 | Me | H | Me | |
| 13-18 | Et | H | Me | |
| 13-19 | Cl | H | Me | |
| 13-20 | OMe | H | Me | |
| 13-21 | CF₃ | H | Me | |
| 13-22 | SO₂Me | H | Me | |
| 13-23 | CH₂OMe | H | Me | |
| 13-24 | CH₂O(CH₂)₂OMe | H | Me | |
| 13-25 | Me | Me | Me | |
| 13-26 | Et | Me | Me | |
| 13-27 | Cl | Me | Me | |
| 13-28 | OMe | Me | Me | |
| 13-29 | CF₃ | Me | Me | |
| 13-30 | SO₂Me | Me | Me | |
| 13-31 | CH₂OMe | Me | Me | |
| 13-32 | CH₂O(CH₂)₂OMe | Me | Me | |

TABLE 14
Inventive compounds of the general formula (I) in which Q is Q1,
R^{X} is an ethyl group and R is hydrogen, and L is the bridge
A8 in which the R⁷ radical is hydrogen

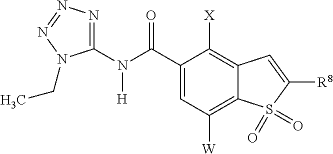

| No. | X | W | R⁸ | Physical data (¹H NMR) |
|---|---|---|---|---|
| 14-1 | Me | H | H | |
| 14-2 | Et | H | H | |
| 14-3 | Cl | H | H | |
| 14-4 | OMe | H | H | |
| 14-5 | CF₃ | H | H | |
| 14-6 | SO₂Me | H | H | |
| 14-7 | CH₂OMe | H | H | |
| 14-8 | CH₂O(CH₂)₂OMe | H | H | |
| 14-9 | Me | Me | H | |
| 14-10 | Et | Me | H | |
| 14-11 | Cl | Me | H | |
| 14-12 | OMe | Me | H | |
| 14-13 | CF₃ | Me | H | |
| 14-14 | SO₂Me | Me | H | |
| 14-15 | CH₂OMe | Me | H | |
| 14-16 | CH₂O(CH₂)₂OMe | Me | H | |
| 14-17 | Me | H | Me | |
| 14-18 | Et | H | Me | |
| 14-19 | Cl | H | Me | |
| 14-20 | OMe | H | Me | |
| 14-21 | CF₃ | H | Me | |
| 14-22 | SO₂Me | H | Me | |
| 14-23 | CH₂OMe | H | Me | |
| 14-24 | CH₂O(CH₂)₂OMe | H | Me | |
| 14-25 | Me | Me | Me | |
| 14-26 | Et | Me | Me | |
| 14-27 | Cl | Me | Me | |
| 14-28 | OMe | Me | Me | |
| 14-29 | CF₃ | Me | Me | |
| 14-30 | SO₂Me | Me | Me | |
| 14-31 | CH₂OMe | Me | Me | |
| 14-32 | CH₂O(CH₂)₂OMe | Me | Me | |

TABLE 15
Inventive compounds of the general formula (I) in which Q is Q1,
R^{X} is an n-propyl group and R is hydrogen, and L is the bridge
A8 in which the R⁷ radical is hydrogen

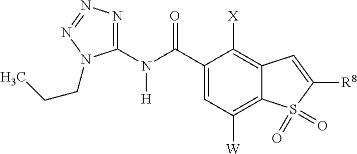

| No. | X | W | R⁸ | Physical data (¹H NMR) |
|---|---|---|---|---|
| 15-1 | Me | H | H | |
| 15-2 | Et | H | H | |
| 15-3 | Cl | H | H | |
| 15-4 | OMe | H | H | |
| 15-5 | CF₃ | H | H | |
| 15-6 | SO₂Me | H | H | |
| 15-7 | CH₂OMe | H | H | |
| 15-8 | CH₂O(CH₂)₂OMe | H | H | |
| 15-9 | Me | Me | H | |
| 15-10 | Et | Me | H | |
| 15-11 | Cl | Me | H | |
| 15-12 | OMe | Me | H | |
| 15-13 | CF₃ | Me | H | |
| 15-14 | SO₂Me | Me | H | |
| 15-15 | CH₂OMe | Me | H | |
| 15-16 | CH₂O(CH₂)₂OMe | Me | H | |
| 15-17 | Me | H | Me | |
| 15-18 | Et | H | Me | |
| 15-19 | Cl | H | Me | |
| 15-20 | OMe | H | Me | |
| 15-21 | CF₃ | H | Me | |
| 15-22 | SO₂Me | H | Me | |
| 15-23 | CH₂OMe | H | Me | |
| 15-24 | CH₂O(CH₂)₂OMe | H | Me | |
| 15-25 | Me | Me | Me | |
| 15-26 | Et | Me | Me | |
| 15-27 | Cl | Me | Me | |
| 15-28 | OMe | Me | Me | |
| 15-29 | CF₃ | Me | Me | |
| 15-30 | SO₂Me | Me | Me | |
| 15-31 | CH₂OMe | Me | Me | |
| 15-32 | CH₂O(CH₂)₂OMe | Me | Me | |

TABLE 16
Inventive compounds of the general formula (I) in which Q is Q2, R^{X}
is a methyl group and R is hydrogen, and L is the bridge A8 in which
the R⁷ radical is hydrogen

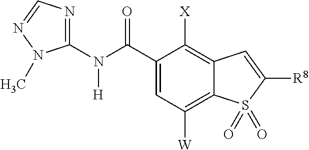

| No. | X | W | R⁸ | Physical data (¹H NMR) |
|---|---|---|---|---|
| 16-1 | Me | H | H | |
| 16-2 | Et | H | H | |
| 16-3 | Cl | H | H | |
| 16-4 | OMe | H | H | |
| 16-5 | CF₃ | H | H | |
| 16-6 | SO₂Me | H | H | |
| 16-7 | CH₂OMe | H | H | |
| 16-8 | CH₂O(CH₂)₂OMe | H | H | |
| 16-9 | Me | Me | H | |
| 16-10 | Et | Me | H | |
| 16-11 | Cl | Me | H | |
| 16-12 | OMe | Me | H | |
| 16-13 | CF₃ | Me | H | |
| 16-14 | SO₂Me | Me | H | |
| 16-15 | CH₂OMe | Me | H | |
| 16-16 | CH₂O(CH₂)₂OMe | Me | H | |
| 16-17 | Me | H | Me | |
| 16-18 | Et | H | Me | |
| 16-19 | Cl | H | Me | |
| 16-20 | OMe | H | Me | |
| 16-21 | CF₃ | H | Me | |
| 16-22 | SO₂Me | H | Me | |
| 16-23 | CH₂OMe | H | Me | |
| 16-24 | CH₂O(CH₂)₂OMe | H | Me | |
| 16-25 | Me | Me | Me | |
| 16-26 | Et | Me | Me | |
| 16-27 | Cl | Me | Me | |
| 16-28 | OMe | Me | Me | |
| 16-29 | CF₃ | Me | Me | |
| 16-30 | SO₂Me | Me | Me | |
| 16-31 | CH₂OMe | Me | Me | |
| 16-32 | CH₂O(CH₂)₂OMe | Me | Me | |

TABLE 17
Inventive compounds of the general formula (I) in which Q is Q3, R^{Y} is a
methyl group and R is hydrogen, and L is the bridge A8 in which the R⁷
radical is hydrogen

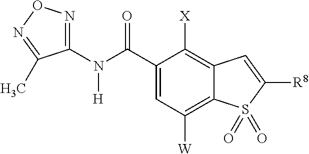

| No. | X | W | R⁸ | Physical data (¹H NMR) |
|---|---|---|---|---|
| 17-1 | Me | H | H | |
| 17-2 | Et | H | H | |
| 17-3 | Cl | H | H | |
| 17-4 | OMe | H | H | |
| 17-5 | CF₃ | H | H | |
| 17-6 | SO₂Me | H | H | |
| 17-7 | CH₂OMe | H | H | |
| 17-8 | CH₂O(CH₂)₂OMe | H | H | |
| 17-9 | Me | Me | H | |
| 17-10 | Et | Me | H | |
| 17-11 | Cl | Me | H | |
| 17-12 | OMe | Me | H | |
| 17-13 | CF₃ | Me | H | |
| 17-14 | SO₂Me | Me | H | |
| 17-15 | CH₂OMe | Me | H | |
| 17-16 | CH₂O(CH₂)₂OMe | Me | H | |
| 17-17 | Me | H | Me | |
| 17-18 | Et | H | Me | |
| 17-19 | Cl | H | Me | (400 MHz, DMSO-d₆ δ, ppm) 8.08 |
| | | | | (d, 1 H), 7.90 (d, 1H), 7.50 (s, 1H), |
| | | | | 2.40 (s, 3H), 2.23 (s, 3H) |
| 17-20 | OMe | H | Me | |
| 17-21 | CF₃ | H | Me | |
| 17-22 | SO₂Me | H | Me | |
| 17-23 | CH₂OMe | H | Me | |
| 17-24 | CH₂O(CH₂)₂OMe | H | Me | |
| 17-25 | Me | Me | Me | |
| 17-26 | Et | Me | Me | |
| 17-27 | Cl | Me | Me | |
| 17-28 | OMe | Me | Me | |
| 17-29 | CF₃ | Me | Me | |
| 17-30 | SO₂Me | Me | Me | |
| 17-31 | CH₂OMe | Me | Me | |
| 17-32 | CH₂O(CH₂)₂OMe | Me | Me | |

TABLE 18
Inventive compounds of the general formula (I) in which Q is Q4, R^{Z} is a
methyl group and R is hydrogen, and L is the bridge A8 in which the R⁷
radical is hydrogen

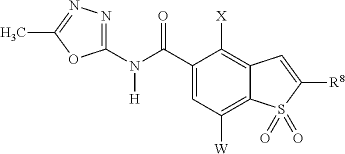

| No. | X | W | R⁸ | Physical data (¹H NMR) |
|---|---|---|---|---|
| 18-1 | Me | H | H | |
| 18-2 | Et | H | H | |
| 18-3 | Cl | H | H | |
| 18-4 | OMe | H | H | |
| 18-5 | CF₃ | H | H | |
| 18-6 | SO₂Me | H | H | |
| 18-7 | CH₂OMe | H | H | |
| 18-8 | CH₂O(CH₂)₂OMe | H | H | |
| 18-9 | Me | Me | H | |
| 18-10 | Et | Me | H | |
| 18-11 | Cl | Me | H | |
| 18-12 | OMe | Me | H | |
| 18-13 | CF₃ | Me | H | |
| 18-14 | SO₂Me | Me | H | |
| 18-15 | CH₂OMe | Me | H | |
| 18-16 | CH₂O(CH₂)₂OMe | Me | H | |
| 18-17 | Me | H | Me | |
| 18-18 | Et | H | Me | |
| 18-19 | Cl | H | Me | |
| 18-20 | OMe | H | Me | |
| 18-21 | CF₃ | H | Me | |
| 18-22 | SO₂Me | H | Me | |
| 18-23 | CH₂OMe | H | Me | |
| 18-24 | CH₂O(CH₂)₂OMe | H | Me | |
| 18-25 | Me | Me | Me | |
| 18-26 | Et | Me | Me | |
| 18-27 | Cl | Me | Me | |
| 18-28 | OMe | Me | Me | |
| 18-29 | CF₃ | Me | Me | |
| 18-30 | SO₂Me | Me | Me | |
| 18-31 | CH₂OMe | Me | Me | |
| 18-32 | CH₂O(CH₂)₂OMe | Me | Me | |

TABLE 19
Inventive compounds of the general formula (I) in which Q is Q1, R^{X} is a
methyl group and R is hydrogen, and L is the bridge A363 in which the
R²⁸, R²⁹, R³⁰ and R³¹ radicals are each hydrogen

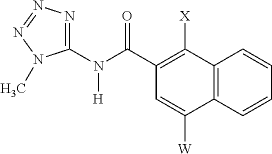

| No. | X | W | Physical data (¹H NMR) |
|---|---|---|---|
| 19-1 | Me | H | |
| 19-2 | Et | H | |
| 19-3 | Cl | H | |
| 19-4 | OMe | H | (400 MHz, DMSO-d₆ δ, ppm) 8.21 (d, 1 H), |
| | | | 7.98 (d, 1 H), 7.76 − 7.61 (m, 4H), 3.98 |
| | | | (s, 3H), 3 35 (s, 3H) |
| 19-5 | CF₃ | H | |
| 19-6 | SO₂Me | H | |
| 19-7 | CH₂OMe | H | |
| 19-8 | CH₂O(CH₂)₂OMe | H | |
| 19-9 | Me | Me | |
| 19-10 | Et | Me | |
| 19-11 | Cl | Me | |
| 19-12 | OMe | Me | |
| 19-13 | CF₃ | Me | |
| 19-14 | SO₂Me | Me | |
| 19-15 | CH₂OMe | Me | |
| 19-16 | CH₂O(CH₂)₂OMe | Me | |

TABLE 20
Inventive compounds of the general formula (I) in which Q is Q1, R^{X} is an
ethyl group and R is hydrogen, and L is the bridge A363 in which the
R²⁸, R²⁹, R³⁰ and R³¹ radicals are each hydrogen

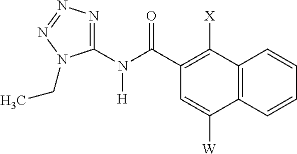

| No. | X | W | Physical data (¹H NMR) |
|---|---|---|---|
| 20-1 | Me | H | |
| 20-2 | Et | H | |
| 20-3 | Cl | H | |
| 20-4 | OMe | H | |
| 20-5 | CF₃ | H | |
| 20-6 | SO₂Me | H | |
| 20-7 | CH₂OMe | H | |
| 20-8 | CH₂O(CH₂)₂OMe | H | |
| 20-9 | Me | Me | |
| 20-10 | Et | Me | |
| 20-11 | Cl | Me | |
| 20-12 | OMe | Me | |
| 20-13 | CF₃ | Me | |
| 20-14 | SO₂Me | Me | |
| 20-15 | CH₂OMe | Me | |
| 20-16 | CH₂O(CH₂)₂OMe | Me | |

TABLE 21
Inventive compounds of the general formula (I) in which Q is Q1, R^{X} is an
n-propyl group and R is hydrogen, and L is the bridge A363 in which the
R²⁸, R²⁹, R³⁰ and R³¹ radicals are each hydrogen

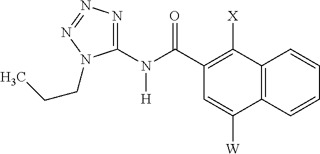

| No. | X | W | Physical data (¹H NMR) |
|---|---|---|---|
| 21-1 | Me | H | |
| 21-2 | Et | H | |
| 21-3 | Cl | H | |
| 21-4 | OMe | H | |
| 21-5 | CF₃ | H | |
| 21-6 | SO₂Me | H | |
| 21-7 | CH₂OMe | H | |
| 21-8 | CH₂O(CH₂)₂OMe | H | |
| 21-9 | Me | Me | |
| 21-10 | Et | Me | |
| 21-11 | Cl | Me | |
| 21-12 | OMe | Me | |
| 21-13 | CF₃ | Me | |
| 21-14 | SO₂Me | Me | |
| 21-15 | CH₂OMe | Me | |
| 21-16 | CH₂O(CH₂)₂OMe | Me | |

TABLE 22
Inventive compounds of the general formula (I) in which Q is Q2, R^{X} is a
methyl group and R is hydrogen, and L is the bridge A363 in which the
R²⁸, R²⁹, R³⁰ and R³¹ radicals are each hydrogen

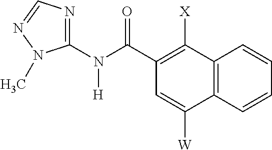

| No. | X | W | Physical data (¹H NMR) |
|---|---|---|---|
| 22-1 | Me | H | |
| 22-2 | Et | H | |
| 22-3 | Cl | H | |
| 22-4 | OMe | H | |
| 22-5 | CF₃ | H | |
| 22-6 | SO₂Me | H | |
| 22-7 | CH₂OMe | H | |
| 22-8 | CH₂O(CH₂)₂OMe | H | |
| 22-9 | Me | Me | |
| 22-10 | Et | Me | |
| 22-11 | Cl | Me | |
| 22-12 | OMe | Me | |
| 22-13 | CF₃ | Me | |
| 22-14 | SO₂Me | Me | |
| 22-15 | CH₂OMe | Me | |
| 22-16 | CH₂O(CH₂)₂OMe | Me | |

TABLE 23
Inventive compounds of the general formula (I) in which Q is Q3, R^{Y} is a
methyl group and R is hydrogen, and L is the bridge A363 in which the
R²⁸, R²⁹, R³⁰ and R³¹ radicals are each hydrogen

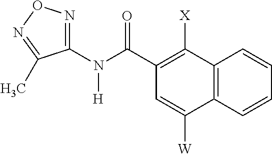

| No. | X | W | Physical data (¹H NMR) |
|---|---|---|---|
| 23-1 | Me | H | |
| 23-2 | Et | H | |
| 23-3 | Cl | H | |
| 23-4 | OMe | H | |
| 23-5 | CF₃ | H | |
| 23-6 | SO₂Me | H | |
| 23-7 | CH₂OMe | H | |
| 23-8 | CH₂O(CH₂)₂OMe | H | |
| 23-9 | Me | Me | |
| 23-10 | Et | Me | |
| 23-11 | Cl | Me | |
| 23-12 | OMe | Me | |
| 23-13 | CF₃ | Me | |
| 23-14 | SO₂Me | Me | |
| 23-15 | CH₂OMe | Me | |
| 23-16 | CH₂O(CH₂)₂OMe | Me | |

TABLE 24
Inventive compounds of the general formula (I) in which Q is Q4, R^{Z} is a
methyl group and R is hydrogen, and L is the bridge A363 in which the
R²⁸, R²⁹, R³⁰ and R³¹ radicals are each hydrogen

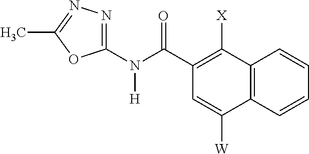

| No. | X | W | Physical data (¹H NMR) |
|---|---|---|---|
| 24-1 | Me | H | |
| 24-2 | Et | H | |
| 24-3 | Cl | H | |
| 24-4 | OMe | H | |
| 24-5 | CF₃ | H | |
| 24-6 | SO₂Me | H | |
| 24-7 | CH₂OMe | H | |
| 24-8 | CH₂O(CH₂)₂OMe | H | |
| 24-9 | Me | Me | |
| 24-10 | Et | Me | |
| 24-11 | Cl | Me | |
| 24-12 | OMe | Me | |
| 24-13 | CF₃ | Me | |
| 24-14 | SO₂Me | Me | |
| 24-15 | CH₂OMe | Me | |
| 24-16 | CH₂O(CH₂)₂OMe | Me | |

TABLE 25
Inventive compounds of the general formula (I) in which Q is Q1, R^{X} is a
methyl group and R is hydrogen, and L is the bridge A364 in which the
R²⁹, R³⁰ and R³¹ radicals are each hydrogen

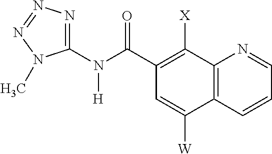

| No. | X | W | Physical data (¹H NMR) |
|---|---|---|---|
| 25-1 | Me | H | |
| 25-2 | Et | H | |
| 25-3 | Cl | H | |
| 25-4 | OMe | H | |
| 25-5 | CF₃ | H | |
| 25-6 | SO₂Me | H | |
| 25-7 | CH₂OMe | H | |
| 25-8 | CH₂O(CH₂)₂OMe | H | |
| 25-9 | Me | Me | |
| 25-10 | Et | Me | |
| 25-11 | Cl | Me | |
| 25-12 | OMe | Me | |
| 25-13 | CF₃ | Me | |
| 25-14 | SO₂Me | Me | |
| 25-15 | CH₂OMe | Me | |
| 25-16 | CH₂O(CH₂)₂OMe | Me | |

TABLE 26
Inventive compounds of the general formula (I) in which Q is Q1, R^{X} is an
ethyl group and R is hydrogen, and L is the bridge A364 in which the R²⁹,
R³⁰ and R³¹ radicals are each hydrogen

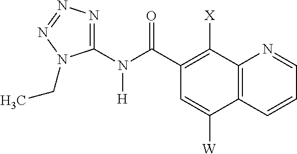

| No. | X | W | Physical data (¹H NMR) |
|---|---|---|---|
| 26-1 | Me | H | |
| 26-2 | Et | H | |
| 26-3 | Cl | H | |
| 26-4 | OMe | H | |
| 26-5 | CF₃ | H | |
| 26-6 | SO₂Me | H | |
| 26-7 | CH₂OMe | H | |
| 26-8 | CH₂O(CH₂)₂OMe | H | |
| 26-9 | Me | Me | |
| 26-10 | Et | Me | |
| 26-11 | Cl | Me | |
| 26-12 | OMe | Me | |
| 26-13 | CF₃ | Me | |
| 26-14 | SO₂Me | Me | |
| 26-15 | CH₂OMe | Me | |
| 26-16 | CH₂O(CH₂)₂OMe | Me | |

TABLE 27
Inventive compounds of the general formula (I) in which Q is Q1, R^{X} is an
n-propyl group and R is hydrogen, and L is the bridge A364 in which the
R²⁹, R³⁰ and R³¹ radicals are each hydrogen

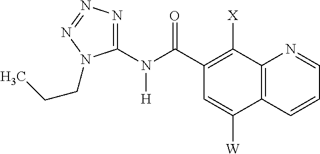

| No. | X | W | Physical data (¹H NMR) |
|---|---|---|---|
| 27-1 | Me | H | |
| 27-2 | Et | H | |
| 27-3 | Cl | H | |
| 27-4 | OMe | H | |
| 27-5 | CF₃ | H | |
| 27-6 | SO₂Me | H | |
| 27-7 | CH₂OMe | H | |
| 27-8 | CH₂O(CH₂)₂OMe | H | |
| 27-9 | Me | Me | |
| 27-10 | Et | Me | |
| 27-11 | Cl | Me | |
| 27-12 | OMe | Me | |
| 27-13 | CF₃ | Me | |
| 27-14 | SO₂Me | Me | |
| 27-15 | CH₂OMe | Me | |
| 27-16 | CH₂O(CH₂)₂OMe | Me | |

TABLE 28
Inventive compounds of the general formula (I) in which Q is Q2, R^{X} is a
methyl group and R is hydrogen, and L is the bridge A364 in which the
R²⁹, R³⁰ and R³¹ radicals are each hydrogen

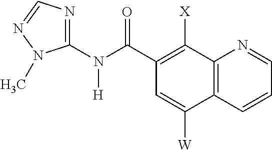

| No. | X | W | Physical data (¹H NMR) |
|---|---|---|---|
| 28-1 | Me | H | |
| 28-2 | Et | H | |
| 28-3 | Cl | H | |
| 28-4 | OMe | H | |
| 28-5 | CF₃ | H | |
| 28-6 | SO₂Me | H | |
| 28-7 | CH₂OMe | H | |
| 28-8 | CH₂O(CH₂)₂OMe | H | |
| 28-9 | Me | Me | |
| 28-10 | Et | Me | |
| 28-11 | Cl | Me | |
| 28-12 | OMe | Me | |
| 28-13 | CF₃ | Me | |
| 28-14 | SO₂Me | Me | |
| 28-15 | CH₂OMe | Me | |
| 28-16 | CH₂O(CH₂)₂OMe | Me | |

TABLE 29
Inventive compounds of the general formula (I) in which Q is Q3, R^{Y} is a
methyl group and R is hydrogen, and L is the bridge A364 in which the
R²⁹, R³⁰ and R³¹ radicals are each hydrogen

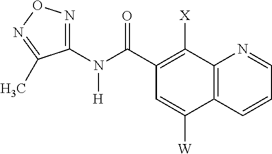

| No. | X | W | Physical data (¹H NMR) |
|---|---|---|---|
| 29-1 | Me | H | |
| 29-2 | Et | H | |
| 29-3 | Cl | H | |
| 29-4 | OMe | H | |
| 29-5 | CF₃ | H | |
| 29-6 | SO₂Me | H | |
| 29-7 | CH₂OMe | H | |
| 29-8 | CH₂O(CH₂)₂OMe | H | |
| 29-9 | Me | Me | |
| 29-10 | Et | Me | |
| 29-11 | Cl | Me | |
| 29-12 | OMe | Me | |
| 29-13 | CF₃ | Me | |
| 29-14 | SO₂Me | Me | |
| 29-15 | CH₂OMe | Me | |
| 29-16 | CH₂O(CH₂)₂OMe | Me | |

TABLE 30
Inventive compounds of the general formula (I) in which Q is Q4, R^{Z} is a
methyl group and R is hydrogen, and L is the bridge A364 in which the
R²⁹, R³⁰ and R³¹ radicals are each hydrogen

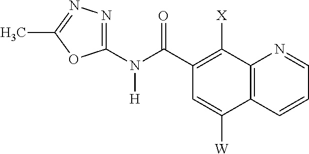

| No. | X | W | Physical data (¹H NMR) |
|---|---|---|---|
| 30-1 | Me | H | |
| 30-2 | Et | H | |
| 30-3 | Cl | H | |
| 30-4 | OMe | H | |
| 30-5 | CF₃ | H | |
| 30-6 | SO₂Me | H | |
| 30-7 | CH₂OMe | H | |
| 30-8 | CH₂O(CH₂)₂OMe | H | |
| 30-9 | Me | Me | |
| 30-10 | Et | Me | |
| 30-11 | Cl | Me | |
| 30-12 | OMe | Me | |
| 30-13 | CF₃ | Me | |
| 30-14 | SO₂Me | Me | |
| 30-15 | CH₂OMe | Me | |
| 30-16 | CH₂O(CH₂)₂OMe | Me | |

TABLE 31
Inventive compounds of the general formula (I) in which Q is Q1, R^{X} is a
methyl group and R is hydrogen, and L is the bridge A365 in which the
R²⁸, R³⁰ and R³¹ radicals are each hydrogen

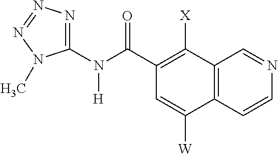

| No. | X | W | Physical data (¹H NMR) |
|---|---|---|---|
| 31-1 | Me | H | |
| 31-2 | Et | H | |
| 31-3 | Cl | H | |
| 31-4 | OMe | H | |
| 31-5 | CF₃ | H | |
| 31-6 | SO₂Me | H | |
| 31-7 | CH₂OMe | H | |
| 31-8 | CH₂O(CH₂)₂OMe | H | |
| 31-9 | Me | Me | |
| 31-10 | Et | Me | |
| 31-11 | Cl | Me | |
| 31-12 | OMe | Me | |
| 31-13 | CF₃ | Me | |
| 31-14 | SO₂Me | Me | |
| 31-15 | CH₂OMe | Me | |
| 31-16 | CH₂O(CH₂)₂OMe | Me | |

TABLE 32
Inventive compounds of the general formula (I) in which Q is Q1, R^{X} is an
ethyl group and R is hydrogen, and L is the bridge A365 in which the R²⁸,
R³⁰ and R³¹ radicals are each hydrogen

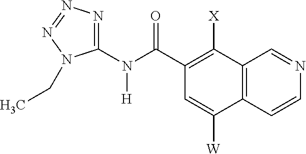

| No. | X | W | Physical data (¹H NMR) |
|---|---|---|---|
| 32-1 | Me | H | |
| 32-2 | Et | H | |
| 32-3 | Cl | H | |
| 32-4 | OMe | H | |
| 32-5 | CF₃ | H | |
| 32-6 | SO₂Me | H | |
| 32-7 | CH₂OMe | H | |
| 32-8 | CH₂O(CH₂)₂OMe | H | |
| 32-9 | Me | Me | |
| 32-10 | Et | Me | |
| 32-11 | Cl | Me | |
| 32-12 | OMe | Me | |
| 32-13 | CF₃ | Me | |
| 32-14 | SO₂Me | Me | |
| 32-15 | CH₂OMe | Me | |
| 32-16 | CH₂O(CH₂)₂OMe | Me | |

TABLE 33
Inventive compounds of the general formula (I) in which Q is Q1, R^{X} is an
n-propyl group and R is hydrogen, and L is the bridge A365 in which the
R²⁸, R³⁰ and R³¹ radicals are each hydrogen

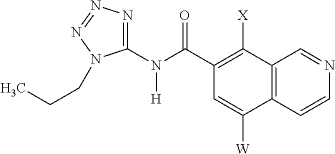

| No. | X | W | Physical data (¹H NMR) |
|---|---|---|---|
| 33-1 | Me | H | |
| 33-2 | Et | H | |
| 33-3 | Cl | H | |
| 33-4 | OMe | H | |
| 33-5 | CF₃ | H | |
| 33-6 | SO₂Me | H | |
| 33-7 | CH₂OMe | H | |
| 33-8 | CH₂O(CH₂)₂OMe | H | |
| 33-9 | Me | Me | |
| 33-10 | Et | Me | |
| 33-11 | Cl | Me | |
| 33-12 | OMe | Me | |
| 33-13 | CF₃ | Me | |
| 33-14 | SO₂Me | Me | |
| 33-15 | CH₂OMe | Me | |
| 33-16 | CH₂O(CH₂)₂OMe | Me | |

TABLE 34
Inventive compounds of the general formula (I) in which Q is Q2, R^{X} is a
methyl group and R is hydrogen, and L is the bridge A365 in which the
R²⁸, R³⁰ and R³¹ radicals are each hydrogen

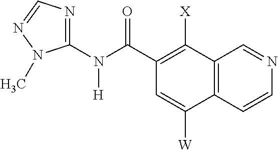

| No. | X | W | Physical data (¹H NMR) |
|---|---|---|---|
| 34-1 | Me | H | |
| 34-2 | Et | H | |
| 34-3 | Cl | H | |
| 34-4 | OMe | H | |
| 34-5 | CF₃ | H | |
| 34-6 | SO₂Me | H | |
| 34-7 | CH₂OMe | H | |
| 34-8 | CH₂O(CH₂)₂OMe | H | |
| 34-9 | Me | Me | |
| 34-10 | Et | Me | |
| 34-11 | Cl | Me | |
| 34-12 | OMe | Me | |
| 34-13 | CF₃ | Me | |
| 34-14 | SO₂Me | Me | |
| 34-15 | CH₂OMe | Me | |
| 34-16 | CH₂O(CH₂)₂OMe | Me | |

TABLE 35
Inventive compounds of the general formula (I) in which Q is Q3, R^{Y} is a
methyl group and R is hydrogen, and L is the bridge A365 in which the
R²⁸, R³⁰ and R³¹ radicals are each hydrogen

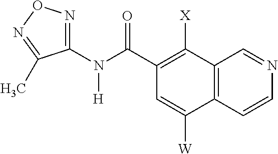

| No. | X | W | Physical data (¹H NMR) |
|---|---|---|---|
| 35-1 | Me | H | |
| 35-2 | Et | H | |
| 35-3 | Cl | H | |
| 35-4 | OMe | H | |
| 35-5 | CF₃ | H | |
| 35-6 | SO₂Me | H | |
| 35-7 | CH₂OMe | H | |
| 35-8 | CH₂O(CH₂)₂OMe | H | |
| 35-9 | Me | Me | |
| 35-10 | Et | Me | |
| 35-11 | Cl | Me | |
| 35-12 | OMe | Me | |
| 35-13 | CF₃ | Me | |
| 35-14 | SO₂Me | Me | |
| 35-15 | CH₂OMe | Me | |
| 35-16 | CH₂O(CH₂)₂OMe | Me | |

TABLE 36
Inventive compounds of the general formula (I) in which Q is Q4, R^{Z} is a
methyl group and R is hydrogen, and L is the bridge A365 in which the
R²⁸, R³⁰ and R³¹ radicals are each hydrogen

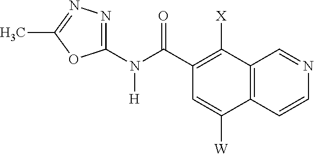

| No. | X | W | Physical data (¹H NMR) |
|---|---|---|---|
| 36-1 | Me | H | |
| 36-2 | Et | H | |
| 36-3 | Cl | H | |
| 36-4 | OMe | H | |
| 36-5 | CF₃ | H | |
| 36-6 | SO₂Me | H | |
| 36-7 | CH₂OMe | H | |
| 36-8 | CH₂O(CH₂)₂OMe | H | |
| 36-9 | Me | Me | |
| 36-10 | Et | Me | |
| 36-11 | Cl | Me | |
| 36-12 | OMe | Me | |
| 36-13 | CF₃ | Me | |
| 36-14 | SO₂Me | Me | |
| 36-15 | CH₂OMe | Me | |
| 36-16 | CH₂O(CH₂)₂OMe | Me | |

TABLE 37
Inventive compounds of the general formula (I) in which Q is Q1, R^{X} is a
methyl group and R is hydrogen, and L is the bridge A366 in which the
R²⁸, R²⁹ and R³¹ radicals are each hydrogen

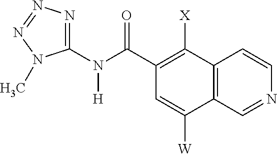

| No. | X | W | Physical data (¹H NMR) |
|---|---|---|---|
| 37-1 | Me | H | |
| 37-2 | Et | H | |
| 37-3 | Cl | H | |
| 37-4 | OMe | H | |
| 37-5 | CF₃ | H | |
| 37-6 | SO₂Me | H | |
| 37-7 | CH₂OMe | H | |
| 37-8 | CH₂O(CH₂)₂OMe | H | |
| 37-9 | Me | Me | |
| 37-10 | Et | Me | |
| 37-11 | Cl | Me | |
| 37-12 | OMe | Me | |
| 37-13 | CF₃ | Me | |
| 37-14 | SO₂Me | Me | |
| 37-15 | CH₂OMe | Me | |
| 37-16 | CH₂O(CH₂)₂OMe | Me | |

TABLE 38
Inventive compounds of the general formula (I) in which Q is Q1, R^{X} is an
ethyl group and R is hydrogen, and L is the bridge A366 in which the R²⁸,
R²⁹ and R³¹ radicals are each hydrogen

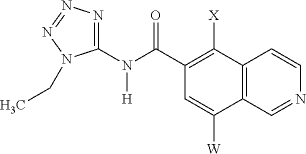

| No. | X | W | Physical data (¹H NMR) |
|---|---|---|---|
| 38-1 | Me | H | |
| 38-2 | Et | H | |
| 38-3 | Cl | H | |
| 38-4 | OMe | H | |
| 38-5 | CF₃ | H | |
| 38-6 | SO₂Me | H | |
| 38-7 | CH₂OMe | H | |
| 38-8 | CH₂O(CH₂)₂OMe | H | |
| 38-9 | Me | Me | |
| 38-10 | Et | Me | |
| 38-11 | Cl | Me | |
| 38-12 | OMe | Me | |
| 38-13 | CF₃ | Me | |
| 38-14 | SO₂Me | Me | |
| 38-15 | CH₂OMe | Me | |
| 38-16 | CH₂O(CH₂)₂OMe | Me | |

TABLE 39
Inventive compounds of the general formula (I) in which Q is Q1, R^{X} is an
n-propyl group and R is hydrogen, and L is the bridge A366 in which the
R²⁸, R²⁹ and R³¹ radicals are each hydrogen

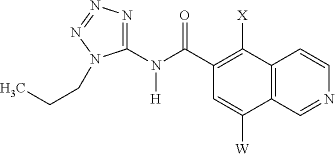

| No. | X | W | Physical data (¹H NMR) |
|---|---|---|---|
| 39-1 | Me | H | |
| 39-2 | Et | H | |
| 39-3 | Cl | H | |
| 39-4 | OMe | H | |
| 39-5 | CF₃ | H | |
| 39-6 | SO₂Me | H | |
| 39-7 | CH₂OMe | H | |
| 39-8 | CH₂O(CH₂)₂OMe | H | |
| 39-9 | Me | Me | |
| 39-10 | Et | Me | |
| 39-11 | Cl | Me | |
| 39-12 | OMe | Me | |
| 39-13 | CF₃ | Me | |
| 39-14 | SO₂Me | Me | |
| 39-15 | CH₂OMe | Me | |
| 39-16 | CH₂O(CH₂)₂OMe | Me | |

TABLE 40
Inventive compounds of the general formula (I) in which Q is Q2, R^{X} is a
methyl group and R is hydrogen, and L is the bridge A366 in which the
R²⁸, R²⁹ and R³¹ radicals are each hydrogen

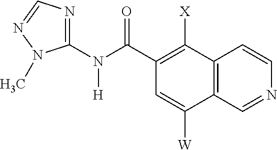

| No. | X | W | Physical data (¹H NMR) |
|---|---|---|---|
| 40-1 | Me | H | |
| 40-2 | Et | H | |
| 40-3 | Cl | H | |
| 40-4 | OMe | H | |
| 40-5 | CF₃ | H | |
| 40-6 | SO₂Me | H | |
| 40-7 | CH₂OMe | H | |
| 40-8 | CH₂O(CH₂)₂OMe | H | |
| 40-9 | Me | Me | |
| 40-10 | Et | Me | |
| 40-11 | Cl | Me | |
| 40-12 | OMe | Me | |
| 40-13 | CF₃ | Me | |
| 40-14 | SO₂Me | Me | |
| 40-15 | CH₂OMe | Me | |
| 40-16 | CH₂O(CH₂)₂OMe | Me | |

TABLE 41
Inventive compounds of the general formula (I) in which Q is Q3, R^{Y} is a
methyl group and R is hydrogen, and L is the bridge A366 in which the
R²⁸, R²⁹ and R³¹ radicals are each hydrogen

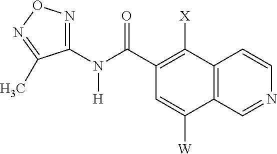

| No. | X | W | Physical data (¹H NMR) |
|---|---|---|---|
| 41-1 | Me | H | |
| 41-2 | Et | H | |
| 41-3 | Cl | H | |
| 41-4 | OMe | H | |
| 41-5 | CF₃ | H | |
| 41-6 | SO₂Me | H | |
| 41-7 | CH₂OMe | H | |
| 41-8 | CH₂O(CH₂)₂OMe | H | |
| 41-9 | Me | Me | |
| 41-10 | Et | Me | |
| 41-11 | Cl | Me | |
| 41-12 | OMe | Me | |
| 41-13 | CF₃ | Me | |
| 41-14 | SO₂Me | Me | |
| 41-15 | CH₂OMe | Me | |
| 41-16 | CH₂O(CH₂)₂OMe | Me | |

TABLE 42
Inventive compounds of the general formula (I) in which Q is Q⁴, R^{Z} is a
methyl group and R is hydrogen, and L is the bridge A366 in which the
R²⁸, R²⁹ and R³¹ radicals are each hydrogen

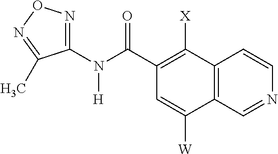

| No. | X | W | Physical data (¹H NMR) |
|---|---|---|---|
| 42-1 | Me | H | |
| 42-2 | Et | H | |
| 42-3 | Cl | H | |
| 42-4 | OMe | H | |
| 42-5 | CF₃ | H | |
| 42-6 | SO₂Me | H | |
| 42-7 | CH₂OMe | H | |
| 42-8 | CH₂O(CH₂)₂OMe | H | |
| 42-9 | Me | Me | |
| 42-10 | Et | Me | |
| 42-11 | Cl | Me | |
| 42-12 | OMe | Me | |
| 42-13 | CF₃ | Me | |
| 42-14 | SO₂Me | Me | |
| 42-15 | CH₂OMe | Me | |
| 42-16 | CH₂O(CH₂)₂OMe | Me | |

TABLE 43
Inventive compounds of the general formula (I) in which Q is Q1, R^{X} is a
methyl group and R is hydrogen, and L is the bridge A367 in which the
R²⁸, R²⁹ and R³⁰ radicals are each hydrogen

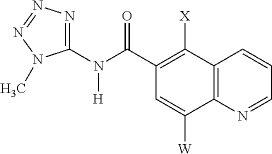

| No. | X | W | Physical data (¹H NMR) |
|---|---|---|---|
| 43-1 | Me | H | |
| 43-2 | Et | H | |
| 43-3 | Cl | H | |
| 43-4 | OMe | H | |
| 43-5 | CF₃ | H | |
| 43-6 | SO₂Me | H | |
| 43-7 | CH₂OMe | H | |
| 43-8 | CH₂O(CH₂)₂OMe | H | |
| 43-9 | Me | Me | |
| 43-10 | Et | Me | |
| 43-11 | Cl | Me | |
| 43-12 | OMe | Me | |
| 43-13 | CF₃ | Me | |
| 43-14 | SO₂Me | Me | |
| 43-15 | CH₂OMe | Me | |
| 43-16 | CH₂O(CH₂)₂OMe | Me | |

TABLE 44
Inventive compounds of the general formula (I) in which Q is Q1, R^{X} is an
ethyl group and R is hydrogen, and L is the bridge A367 in which the R²⁸,
R²⁹ and R³⁰ radicals are each hydrogen

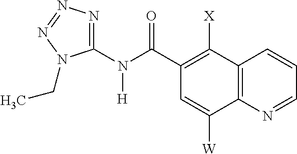

| No. | X | W | Physical data (¹H NMR) |
|---|---|---|---|
| 44-1 | Me | H | |
| 44-2 | Et | H | |
| 44-3 | Cl | H | |
| 44-4 | OMe | H | |
| 44-5 | CF₃ | H | |
| 44-6 | SO₂Me | H | |
| 44-7 | CH₂OMe | H | |
| 44-8 | CH₂O(CH₂)₂OMe | H | |
| 44-9 | Me | Me | |
| 44-10 | Et | Me | |
| 44-11 | Cl | Me | |
| 44-12 | OMe | Me | |
| 44-13 | CF₃ | Me | |
| 44-14 | SO₂Me | Me | |
| 44-15 | CH₂OMe | Me | |
| 44-16 | CH₂O(CH₂)₂OMe | Me | |

TABLE 45
Inventive compounds of the general formula (I) in which Q is Q1, R^{X} is an
n-propyl group and R is hydrogen, and L is the bridge A367 in which the
R²⁸, R²⁹ and R³⁰ radicals are each hydrogen

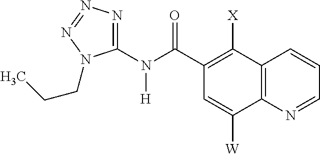

| No. | X | W | Physical data (¹H NMR) |
|---|---|---|---|
| 45-1 | Me | H | |
| 45-2 | Et | H | |
| 45-3 | Cl | H | |
| 45-4 | OMe | H | |
| 45-5 | CF₃ | H | |
| 45-6 | SO₂Me | H | |
| 45-7 | CH₂OMe | H | |
| 45-8 | CH₂O(CH₂)₂OMe | H | |
| 45-9 | Me | Me | |
| 45-10 | Et | Me | |
| 45-11 | Cl | Me | |
| 45-12 | OMe | Me | |
| 45-13 | CF₃ | Me | |
| 45-14 | SO₂Me | Me | |
| 45-15 | CH₂OMe | Me | |
| 45-16 | CH₂O(CH₂)₂OMe | Me | |

TABLE 46
Inventive compounds of the general formula (I) in which Q is Q2, R^{X} is a
methyl group and R is hydrogen, and L is the bridge A367 in which the
R²⁸, R²⁹ and R³⁰ radicals are each hydrogen

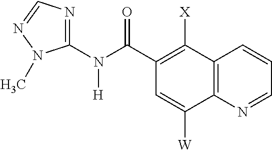

| No. | X | W | Physical data (¹H NMR) |
|---|---|---|---|
| 46-1 | Me | H | |
| 46-2 | Et | H | |
| 46-3 | Cl | H | |
| 46-4 | OMe | H | |
| 46-5 | CF₃ | H | |
| 46-6 | SO₂Me | H | |
| 46-7 | CH₂OMe | H | |
| 46-8 | CH₂O(CH₂)₂OMe | H | |
| 46-9 | Me | Me | |
| 46-10 | Et | Me | |
| 46-11 | Cl | Me | |
| 46-12 | OMe | Me | |
| 46-13 | CF₃ | Me | |
| 46-14 | SO₂Me | Me | |
| 46-15 | CH₂OMe | Me | |
| 46-16 | CH₂O(CH₂)₂OMe | Me | |

TABLE 47
Inventive compounds of the general formula (I) in which Q is Q3, R^{Y} is a
methyl group and R is hydrogen, and L is the bridge A367 in which the
R²⁸, R²⁹ and R³⁰ radicals are each hydrogen

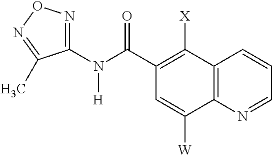

| No. | X | W | Physical data (¹H NMR) |
|---|---|---|---|
| 47-1 | Me | H | |
| 47-2 | Et | H | |
| 47-3 | Cl | H | |
| 47-4 | OMe | H | |
| 47-5 | CF₃ | H | |
| 47-6 | SO₂Me | H | |
| 47-7 | CH₂OMe | H | |
| 47-8 | CH₂O(CH₂)₂OMe | H | |
| 47-9 | Me | Me | |
| 47-10 | Et | Me | |
| 47-11 | Cl | Me | |
| 47-12 | OMe | Me | |
| 47-13 | CF₃ | Me | |
| 47-14 | SO₂Me | Me | |
| 47-15 | CH₂OMe | Me | |
| 47-16 | CH₂O(CH₂)₂OMe | Me | |

TABLE 48
Inventive compounds of the general formula (I) in which Q is Q4, R^{Z} is a
methyl group and R is hydrogen, and L is the bridge A367 in which the
R²⁸, R²⁹ and R³⁰ radicals are each hydrogen

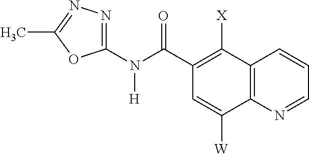

| No. | X | W | Physical data (¹H NMR) |
|---|---|---|---|
| 48-1 | Me | H | |
| 48-2 | Et | H | |
| 48-3 | Cl | H | |
| 48-4 | OMe | H | |
| 48-5 | CF₃ | H | |
| 48-6 | SO₂Me | H | |
| 48-7 | CH₂OMe | H | |
| 48-8 | CH₂O(CH₂)₂OMe | H | |
| 48-9 | Me | Me | |
| 48-10 | Et | Me | |
| 48-11 | Cl | Me | |
| 48-12 | OMe | Me | |
| 48-13 | CF₃ | Me | |
| 48-14 | SO₂Me | Me | |
| 48-15 | CH₂OMe | Me | |
| 48-16 | CH₂O(CH₂)₂OMe | Me | |

TABLE 49
Inventive compounds of the general formula (I) in the form of the sodium
salts, in which Q is Q1, R^{x} is a methyl group, and L is the bridge A30 in
which the R⁷ and R⁸ radicals are both hydrogen

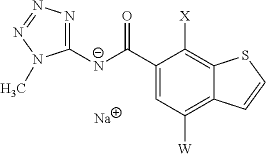

| No. | X | W | Physical data (¹H NMR) |
|---|---|---|---|
| 49-1 | Me | H | |
| 49-2 | Et | H | |
| 49-3 | Cl | H | |
| 49-4 | OMe | H | |
| 49-5 | CF₃ | H | |
| 49-6 | SO₂Me | H | |
| 49-7 | CH₂OMe | H | |
| 49-8 | CH₂O(CH₂)₂OMe | H | |
| 49-9 | Me | Me | |
| 49-10 | Et | Me | |
| 49-11 | Cl | Me | |
| 49-12 | OMe | Me | |
| 49-13 | CF₃ | Me | |
| 49-14 | SO₂Me | Me | |
| 49-15 | CH₂OMe | Me | |
| 49-16 | CH₂O(CH₂)₂OMe | Me | |

TABLE 50
Inventive compounds of the general formula (I) in the form of the sodium
salts, in which Q is Q1, R^{X} is a methyl group, and L is the bridge A32 in
which the R⁷ and R⁸ radicals are both hydrogen

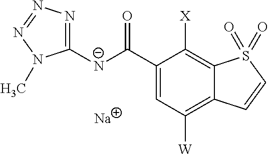

| No. | X | W | Physical data (¹H NMR) |
|---|---|---|---|
| 50-1 | Me | H | |
| 50-2 | Et | H | |
| 50-3 | Cl | H | |
| 50-4 | OMe | H | |
| 50-5 | CF₃ | H | |
| 50-6 | SO₂Me | H | |
| 50-7 | CH₂OMe | H | |
| 50-8 | CH₂O(CH₂)₂OMe | H | |
| 50-9 | Me | Me | |
| 50-10 | Et | Me | |
| 50-11 | Cl | Me | |
| 50-12 | OMe | Me | |
| 50-13 | CF₃ | Me | |
| 50-14 | SO₂Me | Me | |
| 50-15 | CH₂OMe | Me | |
| 50-16 | CH₂O(CH₂)₂OMe | Me | |

TABLE 51
Inventive compounds of the general formula (I) in the form of the
sodium salts, in which Q is Q1, R^{x} is a methyl group, and L is the
bridge A8 in which the R⁷ radical is hydrogen

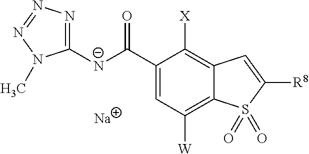

| No. | X | W | R⁸ | Physical data (¹H NMR) |
|---|---|---|---|---|
| 51-1 | Me | H | H | |
| 51-2 | Et | H | H | |
| 51-3 | Cl | H | H | |
| 51-4 | OMe | H | H | |
| 51-5 | CF₃ | H | H | |
| 51-6 | SO₂Me | H | H | |
| 51-7 | CH₂OMe | H | H | |
| 51-8 | CH₂O(CH₂)₂OMe | H | H | |
| 51-9 | Me | Me | H | |
| 51-10 | Et | Me | H | |
| 51-11 | Cl | Me | H | |
| 51-12 | OMe | Me | H | |
| 51-13 | CF₃ | Me | H | |
| 51-14 | SO₂Me | Me | H | |
| 51-15 | CH₂OMe | Me | H | |
| 51-16 | CH₂O(CH₂)₂OMe | Me | H | |
| 51-17 | Me | H | Me | |
| 51-18 | Et | H | Me | |
| 51-19 | Cl | H | Me | |
| 51-20 | OMe | H | Me | |
| 51-21 | CF₃ | H | Me | |
| 51-22 | SO₂Me | H | Me | |
| 51-23 | CH₂OMe | H | Me | |
| 51-24 | CH₂O(CH₂)₂OMe | H | Me | |
| 51-25 | Me | Me | Me | |
| 51-26 | Et | Me | Me | |
| 51-27 | Cl | Me | Me | |
| 51-28 | OMe | Me | Me | |
| 51-29 | CF₃ | Me | Me | |
| 51-30 | SO₂Me | Me | Me | |
| 51-31 | CH₂OMe | Me | Me | |
| 51-32 | CH₂O(CH₂)₂OMe | Me | Me | |

TABLE 52
Inventive compounds of the general formula (I) in the form of the sodium
salts, in which Q is Q1, R^{x} is a methyl group, and L is the bridge A363 in
which the R²⁸, R²⁹, R³⁰ and R³¹ radicals are each hydrogen

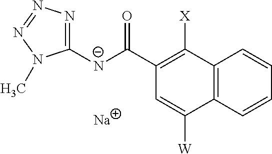

| No. | X | W | Physical data (¹H NMR) |
|---|---|---|---|
| 52-1 | Me | H | |
| 52-2 | Et | H | |
| 52-3 | Cl | H | |
| 52-4 | OMe | H | |
| 52-5 | CF₃ | H | |
| 52-6 | SO₂Me | H | |
| 52-7 | CH₂OMe | H | |
| 52-8 | CH₂O(CH₂)₂OMe | H | |
| 52-9 | Me | Me | |
| 52-10 | Et | Me | |
| 52-11 | Cl | Me | |
| 52-12 | OMe | Me | |
| 52-13 | CF₃ | Me | |
| 52-14 | SO₂Me | Me | |
| 52-15 | CH₂OMe | Me | |
| 52-16 | CH₂O(CH₂)₂OMe | Me | |

TABLE 53
Inventive compounds of the general formula (I) in the form of the sodium
salts, in which Q is Q1, R^{x} is a methyl group, and L is the bridge A364 in
which the R²⁹, R³⁰ and R³¹ radicals are each hydrogen

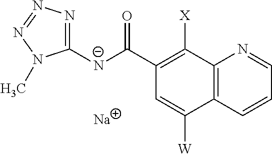

| No. | X | W | Physical data (¹H NMR) |
|---|---|---|---|
| 53-1 | Me | H | |
| 53-2 | Et | H | |
| 53-3 | Cl | H | |
| 53-4 | OMe | H | |
| 53-5 | CF₃ | H | |
| 53-6 | SO₂Me | H | |
| 53-7 | CH₂OMe | H | |
| 53-8 | CH₂O(CH₂)₂OMe | H | |
| 53-9 | Me | Me | |
| 53-10 | Et | Me | |
| 53-11 | Cl | Me | |
| 53-12 | OMe | Me | |
| 53-13 | CF₃ | Me | |
| 53-14 | SO₂Me | Me | |
| 53-15 | CH₂OMe | Me | |
| 53-16 | CH₂O(CH₂)₂OMe | Me | |

TABLE 54
Inventive compounds of the general formula (I) in the form of the sodium
salts, in which Q is Q1, R^{x} is a methyl group, and L is the bridge A365 in
which the R²⁸, R³⁰ and R³¹ radicals are each hydrogen

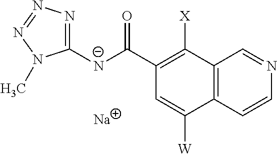

| No. | X | W | Physical data (¹H NMR) |
|---|---|---|---|
| 54-1 | Me | H | |
| 54-2 | Et | H | |
| 54-3 | Cl | H | |
| 54-4 | OMe | H | |
| 54-5 | CF₃ | H | |
| 54-6 | SO₂Me | H | |
| 54-7 | CH₂OMe | H | |
| 54-8 | CH₂O(CH₂)₂OMe | H | |
| 54-9 | Me | Me | |
| 54-10 | Et | Me | |
| 54-11 | Cl | Me | |
| 54-12 | OMe | Me | |
| 54-13 | CF₃ | Me | |
| 54-14 | SO₂Me | Me | |
| 54-15 | CH₂OMe | Me | |
| 54-16 | CH₂O(CH₂)₂OMe | Me | |

TABLE 55
Inventive compounds of the general formula (I) in the form of the sodium
salts, in which Q is Q1, R^{x} is a methyl group, and L is the bridge A366 in
which the R²⁸, R²⁹ and R³¹ radicals are each hydrogen

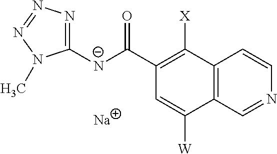

| No. | X | W | Physical data (¹H NMR) |
|---|---|---|---|
| 55-1 | Me | H | |
| 55-2 | Et | H | |
| 55-3 | Cl | H | |
| 55-4 | OMe | H | |
| 55-5 | CF₃ | H | |
| 55-6 | SO₂Me | H | |
| 55-7 | CH₂OMe | H | |
| 55-8 | CH₂O(CH₂)₂OMe | H | |
| 55-9 | Me | Me | |
| 55-10 | Et | Me | |
| 55-11 | Cl | Me | |
| 55-12 | OMe | Me | |
| 55-13 | CF₃ | Me | |
| 55-14 | SO₂Me | Me | |
| 55-15 | CH₂OMe | Me | |
| 55-16 | CH₂O(CH₂)₂OMe | Me | |

TABLE 56
Inventive compounds of the general formula (I) in the form of the sodium
salts, in which Q is Q1, R^{x} is a methyl group, and L is the bridge A367 in
which the R²⁸, R²⁹ and R³⁰ radicals are each hydrogen

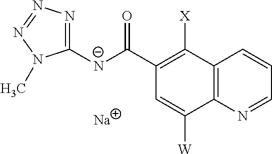

| No. | X | W | Physical data (¹H NMR) |
|---|---|---|---|
| 56-1 | Me | H | |
| 56-2 | Et | H | |
| 56-3 | Cl | H | |
| 56-4 | OMe | H | |
| 56-5 | CF₃ | H | |
| 56-6 | SO₂Me | H | |
| 56-7 | CH₂OMe | H | |
| 56-8 | CH₂O(CH₂)₂OMe | H | |
| 56-9 | Me | Me | |
| 56-10 | Et | Me | |
| 56-11 | Cl | Me | |
| 56-12 | OMe | Me | |
| 56-13 | CF₃ | Me | |
| 56-14 | SO₂Me | Me | |
| 56-15 | CH₂OMe | Me | |
| 56-16 | CH₂O(CH₂)₂OMe | Me | |

TABLE 57
Inventive compounds of the general formula (I) in which Q is Q1 and R is
hydrogen, and L is the bridge A6 in which the R⁷ and R⁸ radicals are
each hydrogen

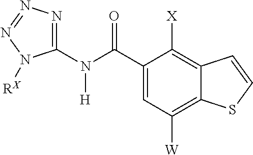

| No. | R^{X} | X | W | Physical data (¹H NMR) |
|---|---|---|---|---|
| 57-1 | Me | Me | H | |
| 57-2 | Me | Et | H | |
| 57-3 | Me | Cl | H | |
| 57-4 | Me | OMe | H | |
| 57-5 | Me | CF₃ | H | |
| 57-6 | Me | SO₂Me | H | |
| 57-7 | Me | CH₂OMe | H | |
| 57-8 | Me | CH₂O(CH₂)₂OMe | H | |
| 57-9 | Me | Me | Me | (400 MHz, DMSO-d₆ δ, ppm) 11.49 (brs, 1H), 7.90 (d, 1H), 7.72 (d, 1H), |
| | | | | 7.49 (s, 1H), 4.01 (s, 3H), 2.69 (s, 3H), 2.55 (s, 3H) |
| 57-10 | Me | Et | Me | |
| 57-11 | Me | Cl | Me | |
| 57-12 | Me | OMe | Me | |
| 57-13 | Me | CF₃ | Me | |
| 57-14 | Me | SO₂Me | Me | |
| 57-15 | Me | CH₂OMe | Me | |
| 57-16 | Me | CH₂O(CH₂)₂OMe | Me | |
| 57-17 | Et | Me | H | |
| 57-18 | Et | Et | H | |
| 57-19 | Et | Cl | H | |
| 57-20 | Et | OMe | H | |
| 57-21 | Et | CF₃ | H | |
| 57-22 | Et | SO₂Me | H | |
| 57-23 | Et | CH₂OMe | H | |
| 57-24 | Et | CH₂O(CH₂)₂OMe | H | |
| 57-25 | Et | Me | Me | (400 MHz, DMSO-d₆ δ, ppm) 11.38 (brs, 1H), 7.90 (d, 1H), 7.72 (d, 1H), |
| | | | | 7.48 (s, 1H), 4.36 (q, 2H), 2.69 (s, 3H), 2.56 (s, 3H), 1.49 (t, 3H) |
| 57-26 | Et | Et | Me | |
| 57-27 | Et | Cl | Me | |
| 57-28 | Et | OMe | Me | |
| 57-29 | Et | CF₃ | Me | |
| 57-30 | Et | SO₂Me | Me | |
| 57-31 | Et | CH₂OMe | Me | |
| 57-32 | Et | CH₂O(CH₂)₂OMe | Me | |

TABLE 58
Inventive compounds of the general formula (I) in which Q is Q3, R^{Y} is
a methyl group and R is hydrogen, and L is the bridge A6 in which the
R⁷ and R⁸ radicals are each hydrogen

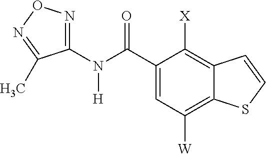

| No. | X | W | Physical data (¹H NMR) |
|---|---|---|---|
| 58-1 | Me | H | |
| 58-2 | Et | H | |
| 58-3 | Cl | H | |
| 58-4 | OMe | H | |
| 58-5 | CF₃ | H | |
| 58-6 | SO₂Me | H | |
| 58-7 | CH₂OMe | H | |
| 58-8 | CH₂O(CH₂)₂OMe | H | |
| 58-9 | Me | Me | (400 MHz, DMSO-d₆ δ, ppm) 7.99 (d, |
| | | | 1H), 7.70 (d, 1H), 7.43 (s, 1H), 3.30 |
| | | | (s, 3H), 2.67 (s, 3H), 2.40 (s, 3H) |
| 58-10 | Et | Me | |
| 58-11 | Cl | Me | |
| 58-12 | OMe | Me | |
| 58-13 | CF₃ | Me | |
| 58-14 | SO₂Me | Me | |
| 58-15 | CH₂OMe | Me | |
| 58-16 | CH₂O(CH₂)₂OMe | Me | |

TABLE 59
Inventive compounds of the general formula (I) in which Q is Q1 and R is
hydrogen, and L is the bridge A8 in which the R⁷ radical is hydrogen

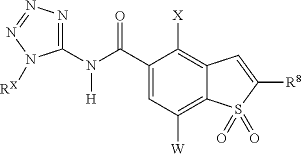

| | | | | | Physical data |
|---|---|---|---|---|---|
| No. | R^{X} | X | W | R⁸ | (¹H NMR) |
| 59-1 | CH₂COOEt | Me | H | H | |
| 59-2 | CH₂COOEt | Et | H | H | |
| 59-3 | CH₂COOEt | Cl | H | H | |
| 59-4 | CH₂COOEt | OMe | H | H | |
| 59-5 | CH₂COOEt | CF₃ | H | H | |
| 59-6 | CH₂COOEt | SO₂Me | H | H | |
| 59-7 | CH₂COOEt | CH₂OMe | H | H | |
| 59-8 | CH₂COOEt | CH₂O(CH₂)₂OMe | H | H | |
| 59-9 | CH₂COOEt | Me | Me | H | |
| 59-10 | CH₂COOEt | Et | Me | H | |
| 59-11 | CH₂COOEt | Cl | Me | H | |
| 59-12 | CH₂COOEt | OMe | Me | H | |
| 59-13 | CH₂COOEt | CF₃ | Me | H | |
| 59-14 | CH₂COOEt | SO₂Me | Me | H | |
| 59-15 | CH₂COOEt | CH₂OMe | Me | H | |
| 59-16 | CH₂COOEt | CH₂O(CH₂)₂OMe | Me | H | |
| 59-17 | CH₂COOEt | Me | H | Me | |
| 59-18 | CH₂COOEt | Et | H | Me | |
| 59-19 | CH₂COOEt | Cl | H | Me | (400 MHz, DMSO-d₆ δ, |
| | | | | | ppm) 7.77 (d, 1H), 7.74 |
| | | | | | (d, 1H), 7.10 (s, 1H), 5.41 |
| | | | | | (s, 2H), 4.27 (q, 2H), 2.20 |
| | | | | | (s, 3H), 1.30 (t, 3H) |
| 59-20 | CH₂COOEt | OMe | H | Me | |
| 59-21 | CH₂COOEt | CF₃ | H | Me | |
| 59-22 | CH₂COOEt | SO₂Me | H | Me | |
| 59-23 | CH₂COOEt | CH₂OMe | H | Me | |
| 59-24 | CH₂COOEt | CH₂O(CH₂)₂OMe | H | Me | |
| 59-25 | CH₂COOEt | Me | Me | Me | |
| 59-26 | CH₂COOEt | Et | Me | Me | |
| 59-27 | CH₂COOEt | Cl | Me | Me | |
| 59-28 | CH₂COOEt | OMe | Me | Me | |
| 59-29 | CH₂COOEt | CF₃ | Me | Me | |
| 59-30 | CH₂COOEt | SO₂Me | Me | Me | |
| 59-31 | CH₂COOEt | CH₂OMe | Me | Me | |
| 59-32 | CH₂COOEt | CH₂O(CH₂)₂OMe | Me | Me | |
| 59-33 | 4-Cl-benzyl | Me | H | H | |
| 59-34 | 4-Cl-benzyl | Et | H | H | |
| 59-35 | 4-Cl-benzyl | Cl | H | H | |
| 59-36 | 4-Cl-benzyl | OMe | H | H | |
| 59-37 | 4-Cl-benzyl | CF₃ | H | H | |
| 59-38 | 4-Cl-benzyl | SO₂Me | H | H | |
| 59-39 | 4-Cl-benzyl | CH₂OMe | H | H | |
| 59-40 | 4-Cl-benzyl | CH₂O(CH₂)₂OMe | H | H | |
| 59-41 | 4-Cl-benzyl | Me | Me | H | |
| 59-42 | 4-Cl-benzyl | Et | Me | H | |
| 59-43 | 4-Cl-benzyl | Cl | Me | H | |
| 59-44 | 4-Cl-benzyl | OMe | Me | H | |
| 59-45 | 4-Cl-benzyl | CF₃ | Me | H | |
| 59-46 | 4-Cl-benzyl | SO₂Me | Me | H | |
| 59-47 | 4-Cl-benzyl | CH₂OMe | Me | H | |
| 59-48 | 4-Cl-benzyl | CH₂O(CH₂)₂OMe | Me | H | |
| 59-49 | 4-Cl-benzyl | Me | H | Me | |
| 59-50 | 4-Cl-benzyl | Et | H | Me | |
| 59-51 | 4-Cl-benzyl | Cl | H | Me | (400 MHz, DMSO-d₆ δ, |
| | | | | | ppm) 8.07 (d, 1H), |
| | | | | | 7.83 (d, 1H), 7.48 |
| | | | | | (d, 2H), 7.32 (d, 2H), |
| | | | | | 5.66 (s, 2H), |
| | | | | | 2.27 (s, 3H) |
| 59-52 | 4-Cl-benzyl | OMe | H | Me | |
| 59-53 | 4-Cl-benzyl | CF₃ | H | Me | |
| 59-54 | 4-Cl-benzyl | SO₂Me | H | Me | |
| 59-55 | 4-Cl-benzyl | CH₂OMe | H | Me | |
| 59-56 | 4-Cl-benzyl | CH₂O(CH₂)₂OMe | H | Me | |
| 59-57 | 4-Cl-benzyl | Me | Me | Me | |
| 59-58 | 4-Cl-benzyl | Et | Me | Me | |
| 59-59 | 4-Cl-benzyl | Cl | Me | Me | |
| 59-60 | 4-Cl-benzyl | OMe | Me | Me | |
| 59-61 | 4-Cl-benzyl | CF₃ | Me | Me | |
| 59-62 | 4-Cl-benzyl | SO₂Me | Me | Me | |
| 59-63 | 4-Cl-benzyl | CH₂OMe | Me | Me | |
| 59-64 | 4-Cl-benzyl | CH₂O(CH₂)₂OMe | Me | Me | |

TABLE 60
Inventive compounds of the general formula (I) in which Q is Q3 and R is
hydrogen, and L is the bridge A8 in which the R⁷ radical is hydrogen

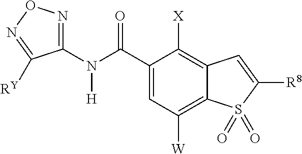

| No. | R^{Y} | X | W | R⁸ | Physical data (¹H NMR) |
|---|---|---|---|---|---|
| 60-1 | Et | Me | H | H | |
| 60-2 | Et | Et | H | H | |
| 60-3 | Et | Cl | H | H | |
| 60-4 | Et | OMe | H | H | |
| 60-5 | Et | CF₃ | H | H | |
| 60-6 | Et | SO₂Me | H | H | |
| 60-7 | Et | CH₂OMe | H | H | |
| 60-8 | Et | CH₂O(CH₂)₂OMe | H | H | |
| 60-9 | Et | Me | Me | H | |
| 60-10 | Et | Et | Me | H | |
| 60-11 | Et | Cl | Me | H | |
| 60-12 | Et | OMe | Me | H | |
| 60-13 | Et | CF₃ | Me | H | |
| 60-14 | Et | SO₂Me | Me | H | |
| 60-15 | Et | CH₂OMe | Me | H | |
| 60-16 | Et | CH₂O(CH₂)₂OMe | Me | H | |
| 60-17 | Et | Me | H | Me | |
| 60-18 | Et | Et | H | Me | |
| 60-19 | Et | Cl | H | Me | (400 MHz, DMSO-d₆ δ, ppm) 8.07 (d, 1H), 7.88 (d, 1H), 7.50 |
| | | | | | (s, 1H), 2.82 (q, 2H), 2.08 (s, 3H), 1.28 (t, 3H) |
| 60-20 | Et | OMe | H | Me | |
| 60-21 | Et | CF₃ | H | Me | |
| 60-22 | Et | SO₂Me | H | Me | |
| 60-23 | Et | CH₂OMe | H | Me | |
| 60-24 | Et | CH₂O(CH₂)₂OMe | H | Me | |
| 60-25 | Et | Me | Me | Me | |
| 60-26 | Et | Et | Me | Me | |
| 60-27 | Et | Cl | Me | Me | |
| 60-28 | Et | OMe | Me | Me | |
| 60-29 | Et | CF₃ | Me | Me | |
| 60-30 | Et | SO₂Me | Me | Me | |
| 60-31 | Et | CH₂OMe | Me | Me | |
| 60-32 | Et | CH₂O(CH₂)₂OMe | Me | Me | |
| 60-33 | OMe | Me | H | H | |
| 60-34 | OMe | Et | H | H | |
| 60-35 | OMe | Cl | H | H | |
| 60-36 | OMe | OMe | H | H | |
| 60-37 | OMe | CF₃ | H | H | |
| 60-38 | OMe | SO₂Me | H | H | |
| 60-39 | OMe | CH₂OMe | H | H | |
| 60-40 | OMe | CH₂O(CH₂)₂OMe | H | H | |
| 60-41 | OMe | Me | Me | H | |
| 60-42 | OMe | Et | Me | H | |
| 60-43 | OMe | Cl | Me | H | |
| 60-44 | OMe | OMe | Me | H | |
| 60-45 | OMe | CF₃ | Me | H | |
| 60-46 | OMe | SO₂Me | Me | H | |
| 60-47 | OMe | CH₂OMe | Me | H | |
| 60-48 | OMe | CH₂O(CH₂)₂OMe | Me | H | |
| 60-49 | OMe | Me | H | Me | |
| 60-50 | OMe | Et | H | Me | |
| 60-51 | OMe | Cl | H | Me | (400 MHz, DMSO-d₆ δ, ppm) 8.03 (d, 1H), 7.80 (d, 1H), 7.48 |
| | | | | | (s, 1H), 4.10 (s, 3H), 2.21 (s, 3H) |
| 60-52 | OMe | OMe | H | Me | |
| 60-53 | OMe | CF₃ | H | Me | |
| 60-54 | OMe | SO₂Me | H | Me | |
| 60-55 | OMe | CH₂OMe | H | Me | |
| 60-56 | OMe | CH₂O(CH₂)₂OMe | H | Me | |
| 60-57 | OMe | Me | Me | Me | |
| 60-58 | OMe | Et | Me | Me | |
| 60-59 | OMe | Cl | Me | Me | |
| 60-60 | OMe | OMe | Me | Me | |
| 60-61 | OMe | CF₃ | Me | Me | |
| 60-62 | OMe | SO₂Me | Me | Me | |
| 60-63 | OMe | CH₂OMe | Me | Me | |
| 60-64 | OMe | CH₂O(CH₂)₂OMe | Me | Me | |
| 60-65 | 4-Cl-phenyl | Me | H | H | |
| 60-66 | 4-Cl-phenyl | Et | H | H | |
| 60-67 | 4-Cl-phenyl | Cl | H | H | |
| 60-68 | 4-Cl-phenyl | OMe | H | H | |
| 60-69 | 4-Cl-phenyl | CF₃ | H | H | |
| 60-70 | 4-Cl-phenyl | SO₂Me | H | H | |
| 60-71 | 4-Cl-phenyl | CH₂OMe | H | H | |
| 60-72 | 4-Cl-phenyl | CH₂O(CH₂)₂OMe | H | H | |
| 60-73 | 4-Cl-phenyl | Me | Me | H | |
| 60-74 | 4-Cl-phenyl | Et | Me | H | |
| 60-75 | 4-Cl-phenyl | Cl | Me | H | |
| 60-76 | 4-Cl-phenyl | OMe | Me | H | |
| 60-77 | 4-Cl-phenyl | CF₃ | Me | H | |
| 60-78 | 4-Cl-phenyl | SO₂Me | Me | H | |
| 60-79 | 4-Cl-phenyl | CH₂OMe | Me | H | |
| 60-80 | 4-Cl-phenyl | CH₂O(CH₂)₂OMe | Me | H | |
| 60-81 | 4-Cl-phenyl | Me | H | Me | |
| 60-82 | 4-Cl-phenyl | Et | H | Me | |
| 60-83 | 4-Cl-phenyl | Cl | H | Me | (400 MHz, DMSO-d₆ δ, ppm) 8.06 (d, 1H), 7.87 (d, 1H), 7.82 |
| | | | | | (d, 2H), 7.43 (s, 1H), 7.66 (d, 2H), 7.48 (s, 1H), 3.31 (s, 3H), |
| | | | | | 2.20 (s, 3H) |
| 60-84 | 4-Cl-phenyl | OMe | H | Me | |
| 60-85 | 4-Cl-phenyl | CF₃ | H | Me | |
| 60-86 | 4-Cl-phenyl | SO₂Me | H | Me | |
| 60-87 | 4-Cl-phenyl | CH₂OMe | H | Me | |
| 60-88 | 4-Cl-phenyl | CH₂O(CH₂)₂OMe | H | Me | |
| 60-89 | 4-Cl-phenyl | Me | Me | Me | |
| 60-90 | 4-Cl-phenyl | Et | Me | Me | |
| 60-91 | 4-Cl-phenyl | Cl | Me | Me | |
| 60-92 | 4-Cl-phenyl | OMe | Me | Me | |
| 60-93 | 4-Cl-phenyl | CF₃ | Me | Me | |
| 60-94 | 4-Cl-phenyl | SO₂Me | Me | Me | |
| 60-95 | 4-Cl-phenyl | CH₂OMe | Me | Me | |
| 60-96 | 4-Cl-phenyl | CH₂O(CH₂)₂OMe | Me | Me | |
| 60-97 | 1,2,4-triazol-1-yl | Me | H | H | |
| 60-98 | 1,2,4-triazol-1-yl | Et | H | H | |
| 60-99 | 1,2,4-triazol-1-yl | Cl | H | H | |
| 60-100 | 1,2,4-triazol-1-yl | OMe | H | H | |
| 60-101 | 1,2,4-triazol-1-yl | CF₃ | H | H | |
| 60-102 | 1,2,4-triazol-1-yl | SO₂Me | H | H | |
| 60-103 | 1,2,4-triazol-1-yl | CH₂OMe | H | H | |
| 60-104 | 1,2,4-triazol-1-yl | CH₂O(CH₂)₂OMe | H | H | |
| 60-105 | 1,2,4-triazol-1-yl | Me | Me | H | |
| 60-106 | 1,2,4-triazol-1-yl | Et | Me | H | |
| 60-107 | 1,2,4-triazol-1-yl | Cl | Me | H | |
| 60-108 | 1,2,4-triazol-1-yl | OMe | Me | H | |
| 60-109 | 1,2,4-triazol-1-yl | CF₃ | Me | H | |
| 60-110 | 1,2,4-triazol-1-yl | SO₂Me | Me | H | |
| 60-111 | 1,2,4-triazol-1-yl | CH₂OMe | Me | H | |
| 60-112 | 1,2,4-triazol-1-yl | CH₂O(CH₂)₂OMe | Me | H | |
| 60-113 | 1,2,4-triazol-1-yl | Me | H | Me | |
| 60-114 | 1,2,4-triazol-1-yl | Et | H | Me | |
| 60-115 | 1,2,4-triazol-1-yl | Cl | H | Me | (400 MHz, DMSO-d₆ δ, ppm) 9.35 (s, 1H), 8.47 (s, 1H), 8.07 |
| | | | | | (d, 1H), 7.86 (d, 1H), 7.48 (s, 1H), 2.20 (s, 3H) |
| 60-116 | 1,2,4-triazol-1-yl | OMe | H | Me | |
| 60-117 | 1,2,4-triazol-1-yl | CF₃ | H | Me | |
| 60-118 | 1,2,4-triazol-1-yl | SO₂Me | H | Me | |
| 60-119 | 1,2,4-triazol-1-yl | CH₂OMe | H | Me | |
| 60-120 | 1,2,4-triazol-1-yl | CH₂O(CH₂)₂OMe | H | Me | |
| 60-121 | 1,2,4-triazol-1-yl | Me | Me | Me | |
| 60-122 | 1,2,4-triazol-1-yl | Et | Me | Me | |
| 60-123 | 1,2,4-triazol-1-yl | Cl | Me | Me | |
| 60-124 | 1,2,4-triazol-1-yl | OMe | Me | Me | |
| 60-125 | 1,2,4-triazol-1-yl | CF₃ | Me | Me | |
| 60-126 | 1,2,4-triazol-1-yl | SO₂Me | Me | Me | |
| 60-127 | 1,2,4-triazol-1-yl | CH₂OMe | Me | Me | |
| 60-128 | 1,2,4-triazol-1-yl | CH₂O(CH₂)₂OMe | Me | Me | |
| 60-129 | t-Bu | Me | H | H | |
| 60-130 | t-Bu | Et | H | H | |
| 60-131 | t-Bu | Cl | H | H | |
| 60-132 | t-Bu | OMe | H | H | |
| 60-133 | t-Bu | CF₃ | H | H | |
| 60-134 | t-Bu | SO₂Me | H | H | |
| 60-135 | t-Bu | CH₂OMe | H | H | |
| 60-136 | t-Bu | CH₂O(CH₂)₂OMe | H | H | |
| 60-137 | t-Bu | Me | Me | H | |
| 60-138 | t-Bu | Et | Me | H | |
| 60-139 | t-Bu | Cl | Me | H | |
| 60-140 | t-Bu | OMe | Me | H | |
| 60-141 | t-Bu | CF₃ | Me | H | |
| 60-142 | t-Bu | SO₂Me | Me | H | |
| 60-143 | t-Bu | CH₂OMe | Me | H | |
| 60-144 | t-Bu | CH₂O(CH₂)₂OMe | Me | H | |
| 60-145 | t-Bu | Me | H | Me | |
| 60-146 | t-Bu | Et | H | Me | |
| 60-147 | t-Bu | Cl | H | Me | (400 MHz, DMSO-d₆ δ, ppm) 8.06 (d, 1H), 7.79 (d, 1H), 7.49 |
| | | | | | (s, 1H), 2.21 (s, 3H), 1.14 (s, 9H) |
| 60-148 | t-Bu | OMe | H | Me | |
| 60-149 | t-Bu | CF₃ | H | Me | |
| 60-150 | t-Bu | SO₂Me | H | Me | |
| 60-151 | t-Bu | CH₂OMe | H | Me | |
| 60-152 | t-Bu | CH₂O(CH₂)₂OMe | H | Me | |
| 60-153 | t-Bu | Me | Me | Me | |
| 60-154 | t-Bu | Et | Me | Me | |
| 60-155 | t-Bu | Cl | Me | Me | |
| 60-156 | t-Bu | OMe | Me | Me | |
| 60-157 | t-Bu | CF₃ | Me | Me | |
| 60-158 | t-Bu | SO₂Me | Me | Me | |
| 60-159 | t-Bu | CH₂OMe | Me | Me | |
| 60-160 | t-Bu | CH₂O(CH₂)₂OMe | Me | Me | |
| 60-161 | c-Pr | Me | H | H | |
| 60-162 | c-Pr | Et | H | H | |
| 60-163 | c-Pr | Cl | H | H | |
| 60-164 | c-Pr | OMe | H | H | |
| 60-165 | c-Pr | CF₃ | H | H | |
| 60-166 | c-Pr | SO₂Me | H | H | |
| 60-167 | c-Pr | CH₂OMe | H | H | |
| 60-168 | c-Pr | CH₂O(CH₂)₂OMe | H | H | |
| 60-169 | c-Pr | Me | Me | H | |
| 60-170 | c-Pr | Et | Me | H | |
| 60-171 | c-Pr | Cl | Me | H | |
| 60-172 | c-Pr | OMe | Me | H | |
| 60-173 | c-Pr | CF₃ | Me | H | |
| 60-174 | c-Pr | SO₂Me | Me | H | |
| 60-175 | c-Pr | CH₂OMe | Me | H | |
| 60-176 | c-Pr | CH₂O(CH₂)₂OMe | Me | H | |
| 60-177 | c-Pr | Me | H | Me | |
| 60-178 | c-Pr | Et | H | Me | |
| 60-179 | c-Pr | Cl | H | Me | (400 MHz, DMSO-d₆ δ, ppm) 8.06 (d, 1H), 7.89 (d, 1H), 7.49 |
| | | | | | (s, 1H), 2.20 (s, 3H), 2.07 (m, 1H), 1.13 (m, 2H), 0.96 (m, 2H) |
| 60-180 | c-Pr | OMe | H | Me | |
| 60-181 | c-Pr | CF₃ | H | Me | |
| 60-182 | c-Pr | SO₂Me | H | Me | |
| 60-183 | c-Pr | CH₂OMe | H | Me | |
| 60-184 | c-Pr | CH₂O(CH₂)₂OMe | H | Me | |
| 60-185 | c-Pr | Me | Me | Me | |
| 60-186 | c-Pr | Et | Me | Me | |
| 60-187 | c-Pr | Cl | Me | Me | |
| 60-188 | c-Pr | OMe | Me | Me | |
| 60-189 | c-Pr | CF₃ | Me | Me | |
| 60-190 | c-Pr | SO₂Me | Me | Me | |
| 60-191 | c-Pr | CH₂OMe | Me | Me | |
| 60-192 | c-Pr | CH₂O(CH₂)₂OMe | Me | Me | |

TABLE 61
Inventive compounds of the general formula (I) in which Q is Q1,
R^{X} is a methyl group and R is hydrogen, and L is the bridge A282
in which the R¹², R¹³, R¹⁴ and R¹⁵ radicals are each hydrogen

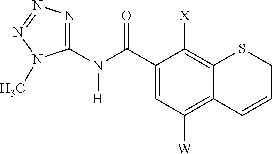

| No. | X | W | Physical data (¹H NMR) |
|---|---|---|---|
| 61-1 | Me | H | (400 MHz, CDCl₃ δ, ppm) 7.39 (d, |
| | | | 1H), 7.03 (d, 1H), 6.53 (dt, 1H), 6.06 |
| | | | (m, 1H), 4.10 (s, 3H), 3.51 (dd, 2H), |
| | | | 2.47 (s, 3H) |
| 61-2 | Et | H | |
| 61-3 | Cl | H | |
| 61-4 | OMe | H | |
| 61-5 | CF₃ | H | |
| 61-6 | SO₂Me | H | |
| 61-7 | CH₂OMe | H | |
| 61-8 | CH₂O(CH₂)₂OMe | H | |
| 61-9 | Me | Me | |
| 61-10 | Et | Me | |
| 61-11 | Cl | Me | |
| 61-12 | OMe | Me | |
| 61-13 | CF₃ | Me | |
| 61-14 | SO₂Me | Me | |
| 61-15 | CH₂OMe | Me | |
| 61-16 | CH₂O(CH₂)₂OMe | Me | |

TABLE 62
Inventive compounds of the general formula (I) in which Q is Q1, R^{X} is
an ethyl group and R is hydrogen, and L is the bridge A282 in which the
R¹², R¹³, R¹⁴ and R¹⁵ radicals are each hydrogen

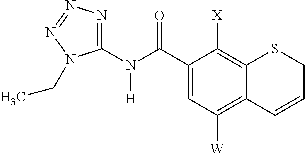

| No. | X | W | Physical data (¹H NMR) |
|---|---|---|---|
| 62-1 | Me | H | |
| 62-2 | Et | H | |
| 62-3 | Cl | H | |
| 62-4 | OMe | H | |
| 62-5 | CF₃ | H | |
| 62-6 | SO₂Me | H | |
| 62-7 | CH₂OMe | H | |
| 62-8 | CH₂O(CH₂)₂OMe | H | |
| 62-9 | Me | Me | |
| 62-10 | Et | Me | |
| 62-11 | Cl | Me | |
| 62-12 | OMe | Me | |
| 62-13 | CF₃ | Me | |
| 62-14 | SO₂Me | Me | |
| 62-15 | CH₂OMe | Me | |
| 62-16 | CH₂O(CH₂)₂OMe | Me | |

TABLE 63
Inventive compounds of the general formula (I) in which Q is Q1, R^{X} is an
n-propyl group and R is hydrogen, and L is the bridge A282 in which the
R¹², R¹³, R¹⁴ and R¹⁵ radicals are each hydrogen

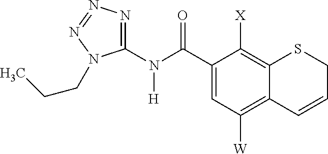

| No. | X | W | Physical data (¹H NMR) |
|---|---|---|---|
| 63-1 | Me | H | |
| 63-2 | Et | H | |
| 63-3 | Cl | H | |
| 63-4 | OMe | H | |
| 63-5 | CF₃ | H | |
| 63-6 | SO₂Me | H | |
| 63-7 | CH₂OMe | H | |
| 63-8 | CH₂O(CH₂)₂OMe | H | |
| 63-9 | Me | Me | |
| 63-10 | Et | Me | |
| 63-11 | Cl | Me | |
| 63-12 | OMe | Me | |
| 63-13 | CF₃ | Me | |
| 63-14 | SO₂Me | Me | |
| 63-15 | CH₂OMe | Me | |
| 63-16 | CH₂O(CH₂)₂OMe | Me | |

TABLE 64
Inventive compounds of the general formula (I) in which Q is Q2, R^{X} is a
methyl group and R is hydrogen, and L is the bridge A282 in which the
R¹², R¹³, R¹⁴ and R¹⁵ radicals are each hydrogen

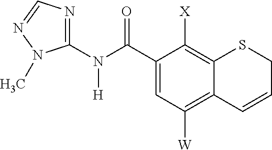

| No. | X | W | Physical data (¹H NMR) |
|---|---|---|---|
| 64-1 | Me | H | |
| 64-2 | Et | H | |
| 64-3 | Cl | H | |
| 64-4 | OMe | H | |
| 64-5 | CF₃ | H | |
| 64-6 | SO₂Me | H | |
| 64-7 | CH₂OMe | H | |
| 64-8 | CH₂O(CH₂)₂OMe | H | |
| 64-9 | Me | Me | |
| 64-10 | Et | Me | |
| 64-11 | Cl | Me | |
| 64-12 | OMe | Me | |
| 64-13 | CF₃ | Me | |
| 64-14 | SO₂Me | Me | |
| 64-15 | CH₂OMe | Me | |
| 64-16 | CH₂O(CH₂)₂OMe | Me | |

TABLE 65
Inventive compounds of the general formula (I) in which Q is Q3, R^{Y} is a
methyl group and R is hydrogen, and L is the bridge A282 in which the
R¹², R¹³, R¹⁴ and R¹⁵ radicals are each hydrogen

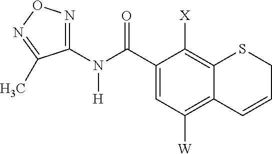

| No. | X | W | Physical data (¹H NMR) |
|---|---|---|---|
| 65-1 | Me | H | |
| 65-2 | Et | H | |
| 65-3 | Cl | H | |
| 65-4 | OMe | H | |
| 65-5 | CF₃ | H | |
| 65-6 | SO₂Me | H | |
| 65-7 | CH₂OMe | H | |
| 65-8 | CH₂O(CH₂)₂OMe | H | |
| 65-9 | Me | Me | |
| 65-10 | Et | Me | |
| 65-11 | Cl | Me | |
| 65-12 | OMe | Me | |
| 65-13 | CF₃ | Me | |
| 65-14 | SO₂Me | Me | |
| 65-15 | CH₂OMe | Me | |
| 65-16 | CH₂O(CH₂)₂OMe | Me | |

TABLE 66
Inventive compounds of the general formula (I) in which Q is Q4, R^{Z} is a
methyl group and R is hydrogen, and L is the bridge A282 in which the
R¹², R¹³, R¹⁴ and R¹⁵ radicals are each hydrogen

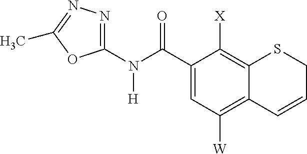

| No. | X | W | Physical data (¹H NMR) |
|---|---|---|---|
| 66-1 | Me | H | |
| 66-2 | Et | H | |
| 66-3 | Cl | H | |
| 66-4 | OMe | H | |
| 66-5 | CF₃ | H | |
| 66-6 | SO₂Me | H | |
| 66-7 | CH₂OMe | H | |
| 66-8 | CH₂O(CH₂)₂OMe | H | |
| 66-9 | Me | Me | |
| 66-10 | Et | Me | |
| 66-11 | Cl | Me | |
| 66-12 | OMe | Me | |
| 66-13 | CF₃ | Me | |
| 66-14 | SO₂Me | Me | |
| 66-15 | CH₂OMe | Me | |
| 66-16 | CH₂O(CH₂)₂OMe | Me | |

TABLE 67
Inventive compounds of the general formula (I) in which Q is Q1, R^{X} is
a methyl group and R is hydrogen, and L is the bridge A286 in which
the R¹², R¹³, R¹⁴ and R¹⁵ radicals are each hydrogen

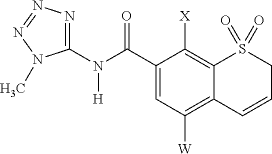

| No. | X | W | Physical data (¹H NMR) |
|---|---|---|---|
| 67-1 | Me | H | (400 MHz, CDCl₃ δ, ppm) 7.82 (d, 1H), |
| | | | 7.30 (d, 1H), 6.72 (d, 1H), 6.26 (m, 1H), |
| | | | 4.13 (s, 3H), 4.08 (dd, 2H), 2.88 (s, 3H) |
| 67-2 | Et | H | |
| 67-3 | Cl | H | |
| 67-4 | OMe | H | |
| 67-5 | CF₃ | H | |
| 67-6 | SO₂Me | H | |
| 67-7 | CH₂OMe | H | |
| 67-8 | CH₂O(CH₂)₂OMe | H | |
| 67-9 | Me | Me | |
| 67-10 | Et | Me | |
| 67-11 | Cl | Me | |
| 67-12 | OMe | Me | |
| 67-13 | CF₃ | Me | |
| 67-14 | SO₂Me | Me | |
| 67-15 | CH₂OMe | Me | |
| 67-16 | CH₂O(CH₂)₂OMe | Me | |

TABLE 68
Inventive compounds of the general formula (I) in which Q is Q1, R^{X} is
an ethyl group and R is hydrogen, and L is the bridge A286 in which the
R¹², R¹³, R¹⁴ and R¹⁵ radicals are each hydrogen

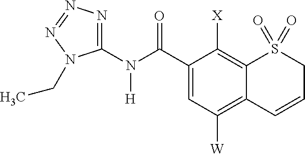

| No. | X | W | Physical data (¹H NMR) |
|---|---|---|---|
| 68-1 | Me | H | |
| 68-2 | Et | H | |
| 68-3 | Cl | H | |
| 68-4 | OMe | H | |
| 68-5 | CF₃ | H | |
| 68-6 | SO₂Me | H | |
| 68-7 | CH₂OMe | H | |
| 68-8 | CH₂O(CH₂)₂OMe | H | |
| 68-9 | Me | Me | |
| 68-10 | Et | Me | |
| 68-11 | Cl | Me | |
| 68-12 | OMe | Me | |
| 68-13 | CF₃ | Me | |
| 68-14 | SO₂Me | Me | |
| 68-15 | CH₂OMe | Me | |
| 68-16 | CH₂O(CH₂)₂OMe | Me | |

TABLE 69
Inventive compounds of the general formula (I) in which Q is Q1, R^{X} is
an n-propyl group and R is hydrogen, and L is the bridge A286 in which
the R¹², R¹³, R¹⁴ and R¹⁵ radicals are each hydrogen

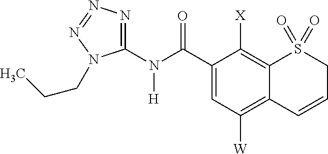

| No. | X | W | Physical data (¹H NMR) |
|---|---|---|---|
| 69-1 | Me | H | |
| 69-2 | Et | H | |
| 69-3 | Cl | H | |
| 69-4 | OMe | H | |
| 69-5 | CF₃ | H | |
| 69-6 | SO₂Me | H | |
| 69-7 | CH₂OMe | H | |
| 69-8 | CH₂O(CH₂)₂OMe | H | |
| 69-9 | Me | Me | |
| 69-10 | Et | Me | |
| 69-11 | Cl | Me | |
| 69-12 | OMe | Me | |
| 69-13 | CF₃ | Me | |
| 69-14 | SO₂Me | Me | |
| 69-15 | CH₂OMe | Me | |
| 69-16 | CH₂O(CH₂)₂OMe | Me | |

TABLE 70
Inventive compounds of the general formula (I) in which Q is Q2, R^{X} is
a methyl group and R is hydrogen, and L is the bridge A286 in which
the R¹², R¹³, R¹⁴ and R¹⁵ radicals are each hydrogen

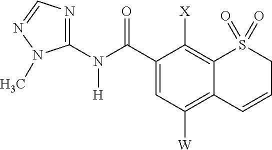

| No. | X | W | Physical data (¹H NMR) |
|---|---|---|---|
| 70-1 | Me | H | |
| 70-2 | Et | H | |
| 70-3 | Cl | H | |
| 70-4 | OMe | H | |
| 70-5 | CF₃ | H | |
| 70-6 | SO₂Me | H | |
| 70-7 | CH₂OMe | H | |
| 70-8 | CH₂O(CH₂)₂OMe | H | |
| 70-9 | Me | Me | |
| 70-10 | Et | Me | |
| 70-11 | Cl | Me | |
| 70-12 | OMe | Me | |
| 70-13 | CF₃ | Me | |
| 70-14 | SO₂Me | Me | |
| 70-15 | CH₂OMe | Me | |
| 70-16 | CH₂O(CH₂)₂OMe | Me | |

TABLE 71
Inventive compounds of the general formula (I) in which Q is Q3, R^{Y} is a
methyl group and R is hydrogen, and L is the bridge A286 in which the
R¹², R¹³, R¹⁴ and R¹⁵ radicals are each hydrogen

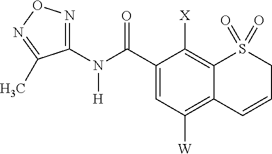

| No. | X | W | Physical data (¹H NMR) |
|---|---|---|---|
| 71-1 | Me | H | |
| 71-2 | Et | H | |
| 71-3 | Cl | H | |
| 71-4 | OMe | H | |
| 71-5 | CF₃ | H | |
| 71-6 | SO₂Me | H | |
| 71-7 | CH₂OMe | H | |
| 71-8 | CH₂O(CH₂)₂OMe | H | |
| 71-9 | Me | Me | |
| 71-10 | Et | Me | |
| 71-11 | Cl | Me | |
| 71-12 | OMe | Me | |
| 71-13 | CF₃ | Me | |
| 71-14 | SO₂Me | Me | |
| 71-15 | CH₂OMe | Me | |
| 71-16 | CH₂O(CH₂)₂OMe | Me | |

TABLE 72
Inventive compounds of the general formula (I) in which Q is Q4, R^{Z} is a
methyl group and R is hydrogen, and L is the bridge A286 in which the
R¹², R¹³, R¹⁴ and R¹⁵ radicals are each hydrogen

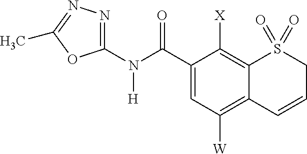

| No. | X | W | Physical data (¹H NMR) |
|---|---|---|---|
| 72-1 | Me | H | |
| 72-2 | Et | H | |
| 72-3 | Cl | H | |
| 72-4 | OMe | H | |
| 72-5 | CF₃ | H | |
| 72-6 | SO₂Me | H | |
| 72-7 | CH₂OMe | H | |
| 72-8 | CH₂O(CH₂)₂OMe | H | |
| 72-9 | Me | Me | |
| 72-10 | Et | Me | |
| 72-11 | Cl | Me | |
| 72-12 | OMe | Me | |
| 72-13 | CF₃ | Me | |
| 72-14 | SO₂Me | Me | |
| 72-15 | CH₂OMe | Me | |
| 72-16 | CH₂O(CH₂)₂OMe | Me | |

TABLE 73
Inventive compounds of the general formula (I) in which Q is Q3, R^{Y} is
chlorine and R is hydrogen, and L is the bridge A282 in which the R¹²,
R¹³, R¹⁴ and R¹⁵ radicals are each hydrogen

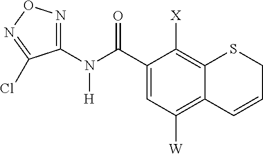

| No. | X | W | Physical data (¹H NMR) |
|---|---|---|---|
| 73-1 | Me | H | |
| 73-2 | Et | H | |
| 73-3 | Cl | H | |
| 73-4 | OMe | H | |
| 73-5 | CF₃ | H | |
| 73-6 | SO₂Me | H | |
| 73-7 | CH₂OMe | H | |
| 73-8 | CH₂O(CH₂)₂OMe | H | |
| 73-9 | Me | Me | |
| 73-10 | Et | Me | |
| 73-11 | Cl | Me | |
| 73-12 | OMe | Me | |
| 73-13 | CF₃ | Me | |
| 73-14 | SO₂Me | Me | |
| 73-15 | CH₂OMe | Me | |
| 73-16 | CH₂O(CH₂)₂OMe | Me | |

TABLE 74
Inventive compounds of the general formula (I) in which Q is Q3, R^{Y} is
chlorine and R is hydrogen, and L is the bridge A286 in which the R¹²,
R¹³, R¹⁴ and R¹⁵ radicals are each hydrogen

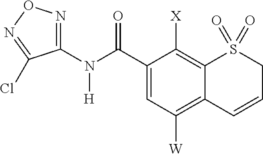

| No. | X | W | Physical data (¹H NMR) |
|---|---|---|---|
| 74-1 | Me | H | |
| 74-2 | Et | H | |
| 74-3 | Cl | H | |
| 74-4 | OMe | H | |
| 74-5 | CF₃ | H | |
| 74-6 | SO₂Me | H | |
| 74-7 | CH₂OMe | H | |
| 74-8 | CH₂O(CH₂)₂OMe | H | |
| 74-9 | Me | Me | |
| 74-10 | Et | Me | |
| 74-11 | Cl | Me | |
| 74-12 | OMe | Me | |
| 74-13 | CF₃ | Me | |
| 74-14 | SO₂Me | Me | |
| 74-15 | CH₂OMe | Me | |
| 74-16 | CH₂O(CH₂)₂OMe | Me | |

### B. FORMULATION EXAMPLES

a) A dusting product is obtained by mixing 10 parts by weight of a compound of the formula (I) and/or salts thereof and 90 parts by weight of talc as an inert substance and comminuting the mixture in a hammer mill.
b) A readily water-dispersible, wettable powder is obtained by mixing 25 parts by weight of a compound of the formula (I) and/or salts thereof, 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277 C), and grinding the mixture in a ball mill to a fineness of below 5 microns.
d) An emulsifiable concentrate is obtained from 15 parts by weight of a compound of the formula (I) and/or salts thereof, 75 parts by weight of cyclohexanone as a solvent and 10 parts by weight of ethoxylated nonylphenol as an emulsifier.
e) Water-dispersible granules are obtained by mixing
   75 parts by weight of a compound of the formula (I) and/or salts thereof,
   10 parts by weight of calcium lignosulfonate,
   5 parts by weight of sodium laurylsulfate,
   3 parts by weight of polyvinyl alcohol and
   7 parts by weight of kaolin,
   grinding the mixture in a pinned-disk mill, and granulating the powder in a fluidized bed by spray application of water as a granulating liquid.
f) Water-dispersible granules are also obtained by homogenizing and precomminuting, in a colloid mill,
   25 parts by weight of a compound of the formula (I) and/or salts thereof,
   5 parts by weight of sodium 2,2′-dinaphthylmethane-6,6′-disulfonate,
   2 parts by weight of sodium oleoylmethyltaurate,
   1 part by weight of polyvinyl alcohol,
   17 parts by weight of calcium carbonate and
   50 parts by weight of water,
   then grinding the mixture in a bead mill and atomizing and drying the resulting suspension in a spray tower by means of a one-phase nozzle.

### C. BIOLOGICAL EXAMPLES

### 1. Pre-Emergence Herbicidal Action Against Harmful Plants

Seeds of monocotyledonous and dicotyledonous weed plants and crop plants are laid out in wood-fiber pots in sandy loam and covered with soil. The inventive compounds, formulated in the form of wettable powders (WP) or as emulsion concentrates (EC), are then applied to the surface of the soil cover in the form of an aqueous suspension or emulsion at a water application rate equating to 600 to 800 l/ha, with addition of 0.2% wetting agent. After the treatment, the pots are placed in a greenhouse and kept under good growth conditions for the test plants. The damage to the trial plants is scored visually after a test period of 3 weeks by comparison with untreated controls (herbicidal activity in percent (%): 100% efficacy=the plants have died, 0% efficacy=like control plants). In this test, for example, compounds no. 17-19, 19-4, 57-25, 60-19, 60-51, 60-147, 61-1 and 67-1 at an application rate of 320 g/ha showed at least 80% efficacy against Veronica persica. Compounds no. 1-1, 17-19 and 60-115 at an application rate of 320 g/ha showed at least 80% efficacy against Polygonum convolvulus, and did not cause any damage at all in corn and wheat. Compounds no. 7-1 and 61-1 at an application rate of 320 g/ha showed at least 80% efficacy against Cyperus serotinus, and did not cause any damage at all in wheat.

### 2. Post-Emergence Herbicidal Action Against Harmful Plants

Seeds of monocotyledonous and dicotyledonous weed and crop plants are laid out in sandy loam in wood-fiber pots, covered with soil and cultivated in a greenhouse under good growth conditions. 2 to 3 weeks after sowing, the test plants are treated at the one-leaf stage. The inventive compounds, formulated in the form of wettable powders (WP) or as emulsion concentrates (EC), are then sprayed as aqueous suspension or emulsion at a water application rate equating to 600 to 800 I/ha with the addition of 0.2% of wetting agent onto the green parts of the plants. After the trial plants have been left to stand in a greenhouse under optimal growth conditions for about 3 weeks, the efficacy of the formulations is scored visually in comparison to untreated controls (herbicidal action in percent (%): 100% efficacy=the plants have died, 0% efficacy=like control plants). In this test, for example, compounds no. 60-19, 60-147 and 67-1 at an application rate of 80 g/ha showed at least 80% efficacy against Veronica persica. Compounds no. 59-51 and 67-1 at an application rate of 80 g/ha showed at least 80% efficacy against Abutilon theophrasti and Pharbitis purpureum, and did not cause any damage at all in corn and wheat. Compounds no. 17-19 and 61-1 at an application rate of 80 g/ha showed at least 80% efficacy against Amaranthus retroflexus, and did not cause any damage at all in rice and wheat.

1. An N-(tetrazol-5-yl)-, N-(triazol-5-yl)-, N-(1,2,5-oxadiazol-3-yl)- or N-(1,3,4-oxadiazol-2-yl)bicycloarylcarboxamide of formula (I) and/or a salt thereof 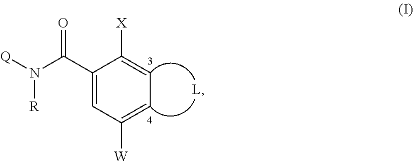
where symbols and indices are each defined as follows:
Q is a Q1, Q2, Q3 or Q4 radical 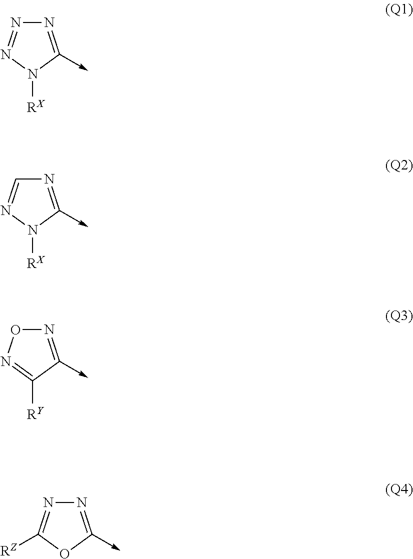
X is nitro, halogen, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R¹O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups,
W is hydrogen, halogen, nitro, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, halo-(C₃-C₇)-cycloalkyl, (C₁-C₆)-alkoxy, halo-(C₁-C₆)-alkoxy, (C₁-C₆)-alkyl-(O)ₙS—, (C₁-C₆)-haloalkyl-(O)ₙS—, (C₁-C₆)-alkoxy-(C₁-C₄)-alkyl, (C₁-C₆)-alkoxy-(C₁-C₄)-haloalkyl, R¹(O)C, R¹(R¹ON═)C, R¹O(O)C, (R¹)₂N, R¹(O)C(R¹)N or R²(O)₂S(R¹)N,
R is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C, R¹O(O)C, (R¹)₂N(O)C, R¹O, (R¹)₂N, R²O(O)C(R¹)N, (R¹)₂N(O)C(R¹)N, R²(O)₂S,
or benzyl substituted in each case by s radicals from the group consisting of methyl, ethyl, methoxy, nitro, trifluoromethyl and halogen,
R^{X} is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of nitro, cyano, (R⁶)₃Si, (R⁵O)₂(O)P, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, and where the four latter radicals are each substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,
or
R^{X} is (C₃-C₇)-cycloalkyl, heteroaryl, heterocyclyl or phenyl, where the four aforementioned radicals are each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl,
R^{Y} is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₇)-cycloalkyl, (C₁-C₆)-alkoxy, halo-(C₁-C₆)-alkoxy, (C₂-C₆)-alkenyloxy, (C₂-C₆)-alkynyloxy, cyano, nitro, methylsulfenyl, methylsulfinyl, methylsulfonyl, acetylamino, benzoylamino, methoxycarbonyl, ethoxycarbonyl, methoxycarbonylmethyl, ethoxycarbonylmethyl, benzoyl, methylcarbonyl, piperidinylcarbonyl, trifluoromethylcarbonyl, halogen, amino, aminocarbonyl, methylaminocarbonyl, dimethylaminocarbonyl, methoxymethyl, or heteroaryl, heterocyclyl or phenyl, each of which is substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,
R^{Z} is hydrogen, (C₁-C₆)-alkyl, R¹O—(C₁-C₆)-alkyl, R¹CH₂, (C₃-C₇)-cycloalkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, R¹O, R¹(H)N, methoxycarbonyl, ethoxycarbonyl, methylcarbonyl, dimethylamino, trifluoromethylcarbonyl, acetylamino, methylsulfenyl, methylsulfinyl, methylsulfonyl, or heteroaryl, heterocyclyl, benzyl oder phenyl each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, where heterocyclyl bears n oxo groups,
R¹ is hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl, heterocyclyl-S(O)ₙ—(C₁-C₆)-alkyl, where the fifteen latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl bears n oxo groups,
R² is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, (C₃-C₆)-cycloalkenyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, phenyl-N(R³)—(C₁-C₆)-alkyl, heteroaryl-N(R³)—(C₁-C₆)-alkyl, heterocyclyl-N(R³)—(C₁-C₆)-alkyl, phenyl-S(O)ₙ—(C₁-C₆)-alkyl, heteroaryl-S(O)ₙ—(C₁-C₆)-alkyl, heterocyclyl-S(O)ₙ—(C₁-C₆)-alkyl, where the fifteen latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R³O(O)C, (R³)₂N(O)C, R³O, (R³)₂N, R⁴(O)ₙS, R³O(O)₂S, (R³)₂N(O)₂S and R³O—(C₁-C₆)-alkyl, and where heterocyclyl bears n oxo groups,
R³ is hydrogen, (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,
R⁴ is (C₁-C₆)-alkyl, halo-(C₁-C₆)-alkyl, (C₂-C₆)-alkenyl, (C₂-C₆)-alkynyl, (C₃-C₆)-cycloalkyl, (C₃-C₆)-cycloalkyl-(C₁-C₆)-alkyl or phenyl,
R⁵ is hydrogen or (C₁-C₄)-alkyl,
R⁶ is (C₁-C₄)-alkyl,
R′ is acetoxy, acetamido, N-methylacetamido, benzoyloxy, benzamido, N-methylbenzamido, methoxycarbonyl, ethoxycarbonyl, benzoyl, methylcarbonyl, piperidinylcarbonyl, morpholinylcarbonyl, trifluoromethylcarbonyl, aminocarbonyl, methylaminocarbonyl, dimethylaminocarbonyl, (C₃-C₆)-cycloalkyl, or heteroaryl or heterocyclyl each substituted by s radicals from the group consisting of methyl, ethyl, methoxy, trifluoromethyl and halogen;
n is 0, 1 or 2,
m is 0, 1, 2, 3 or 4,
s is 0, 1, 2 or 3.
t is 0, 1, 2, 3, 4 or 5,
L is a 3-, 4- or 5-membered fused-on unsaturated bridge wherein the bridge atoms consist of t carbon atoms and m heteroatoms from the group consisting of O, S and N.

2. An N-(tetrazol-5-yl)-, N-(triazol-5-yl)-, N-(1,2,5-oxadiazol-3-yl)- or N-(1,3,4-oxadiazol-2-yl)bicycloarylcarboxamide and/or salt thereof as claimed in claim 1, in which the L bridge represents the A1 to A378 radicals, where dotted bonds represent bonds that bind the L bridge to the benzoyl radical, the upper dotted line represents the bond to carbon atom 3 in the formula (I), and the lower dotted line, the bond to carbon atom 4 in the formula (I): 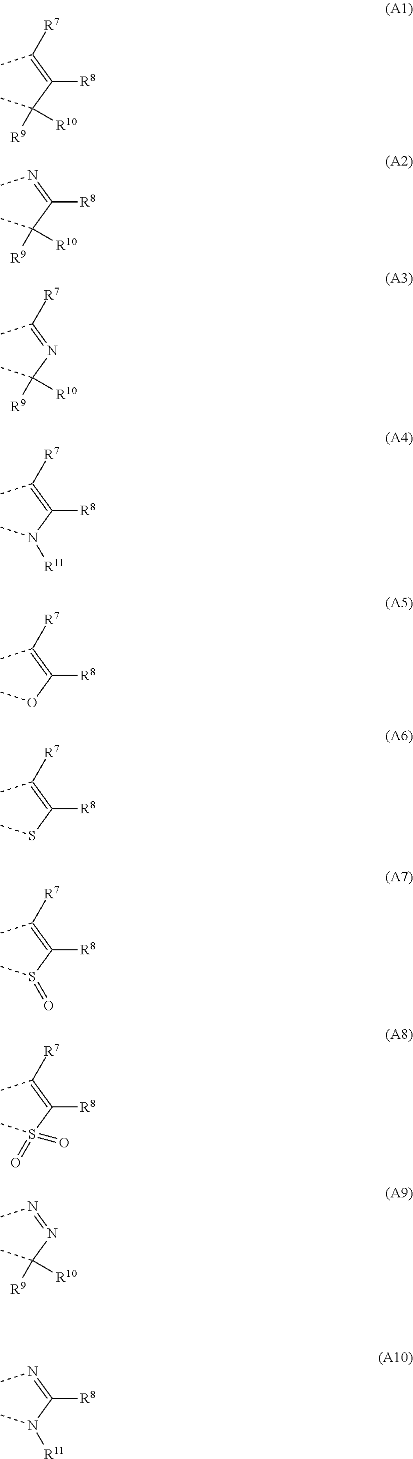 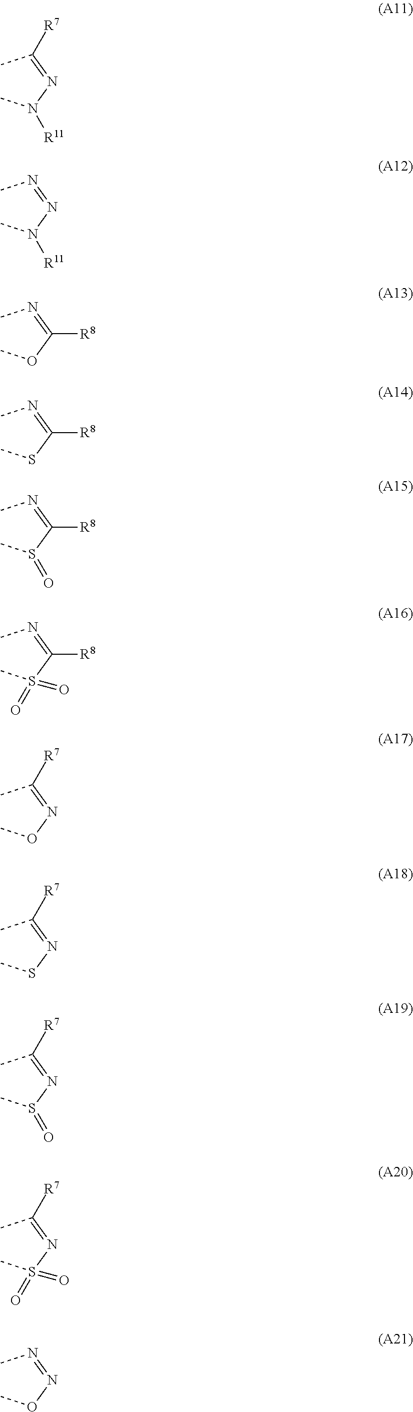 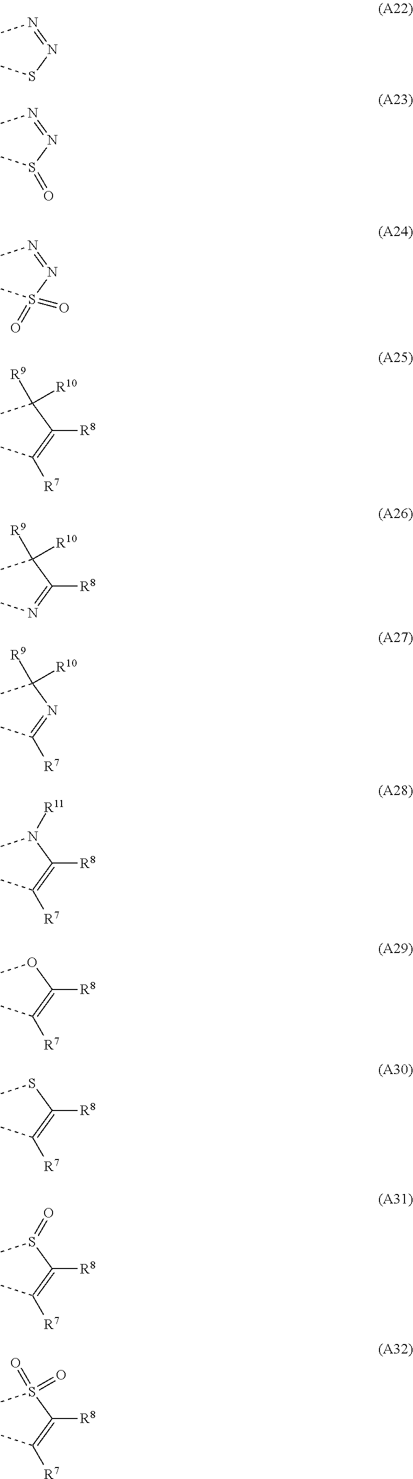 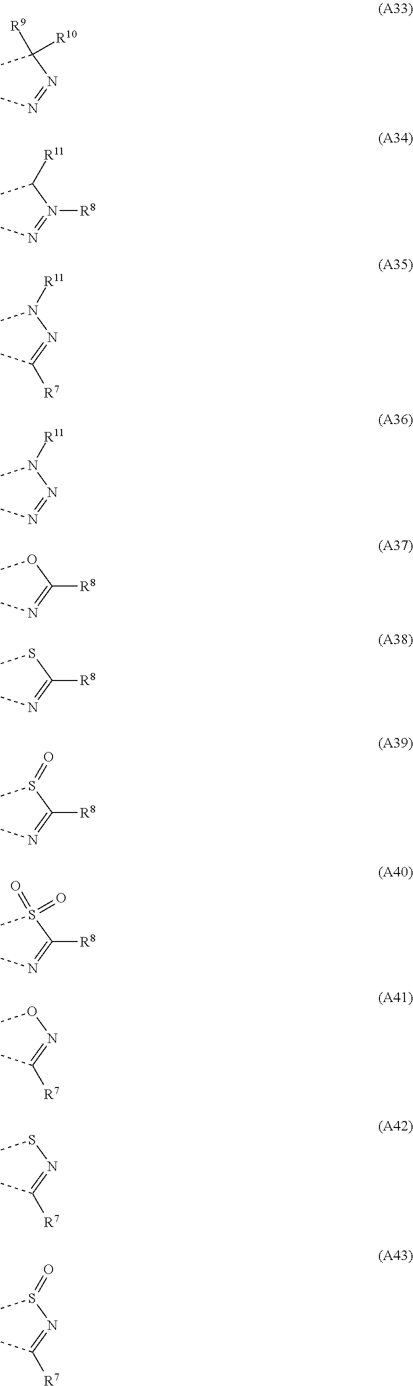 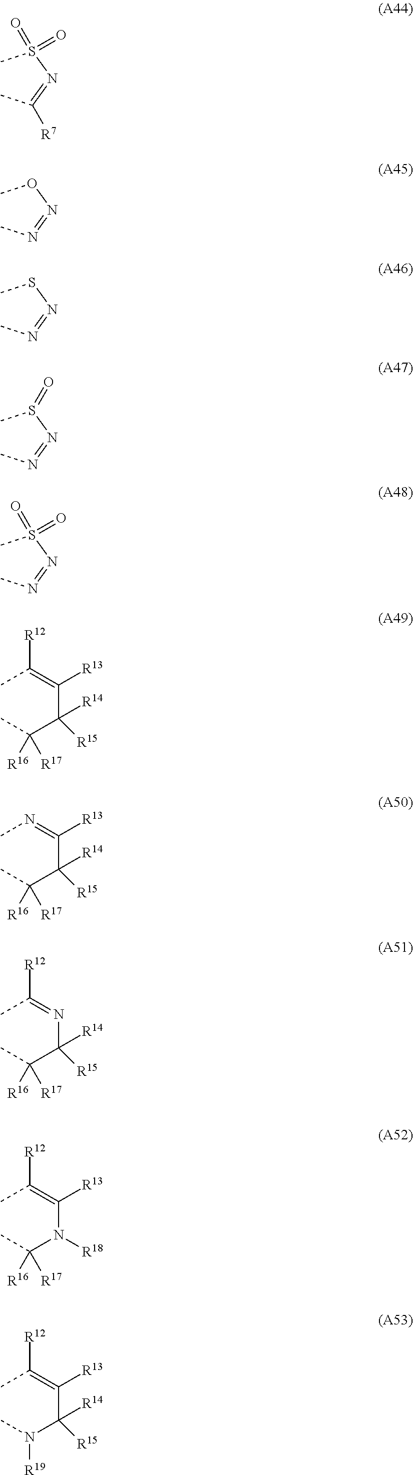 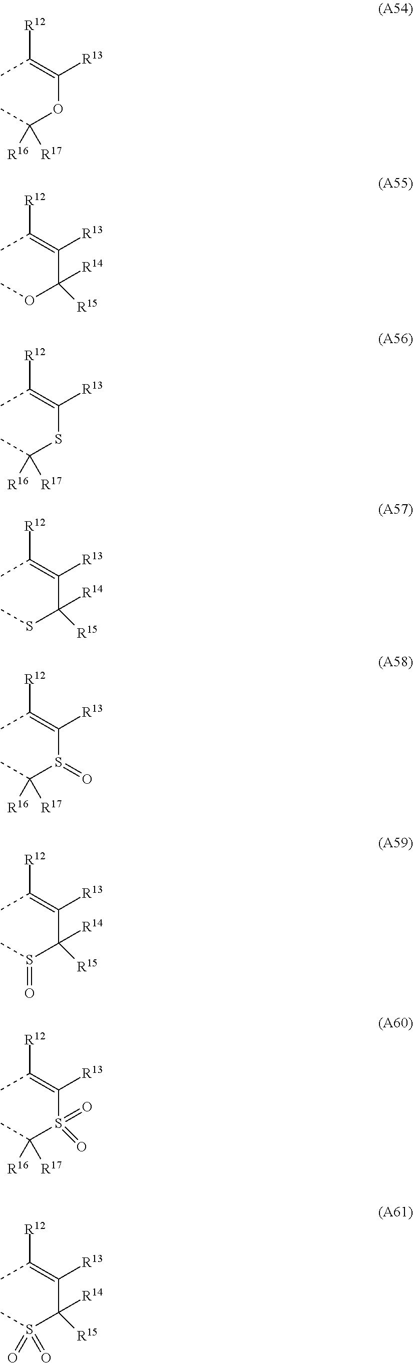 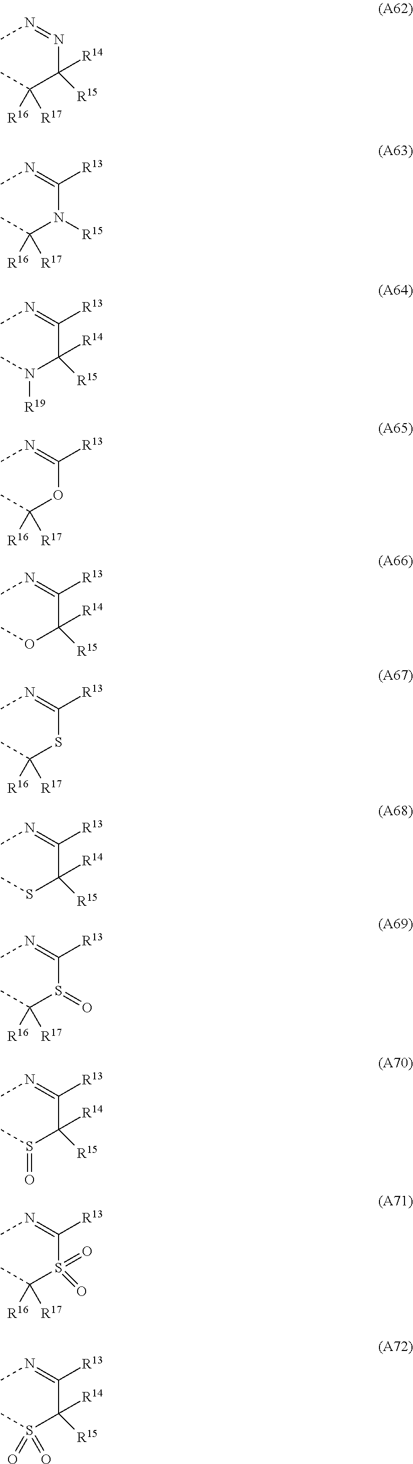 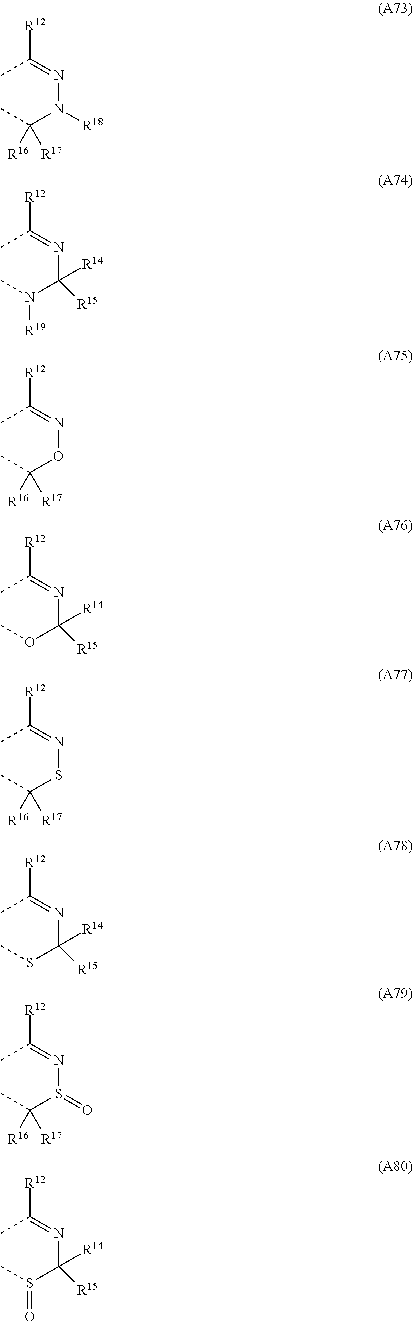 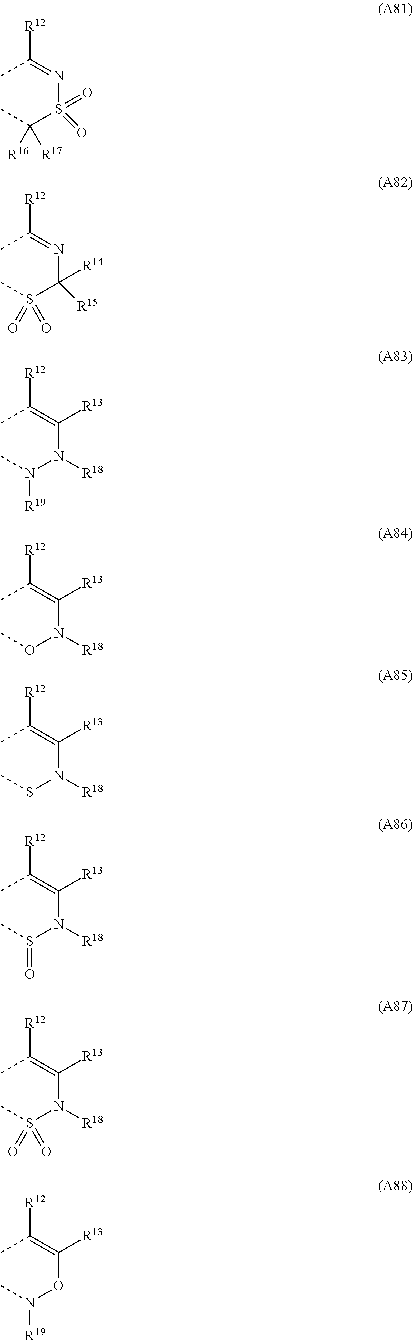 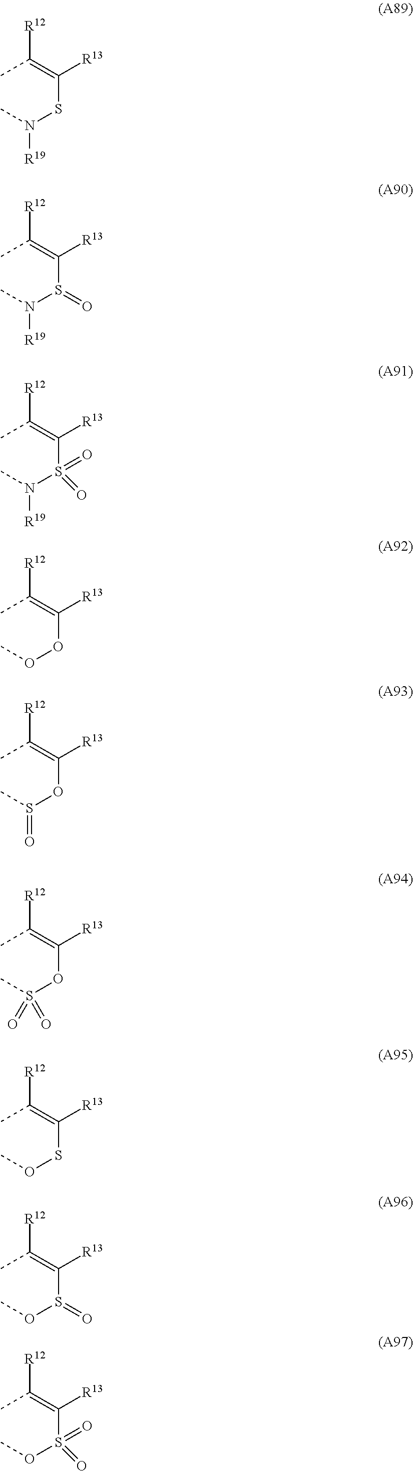 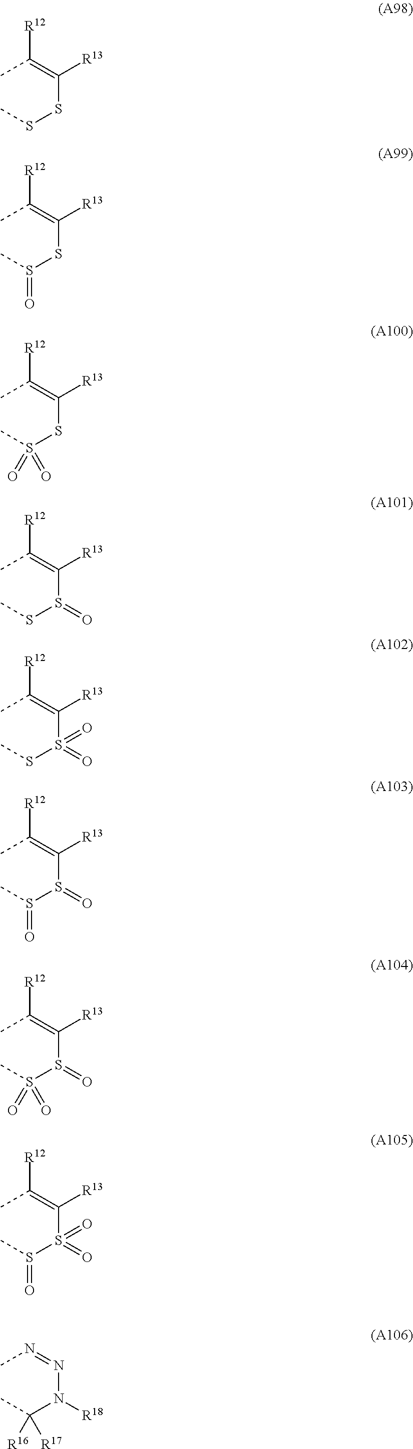 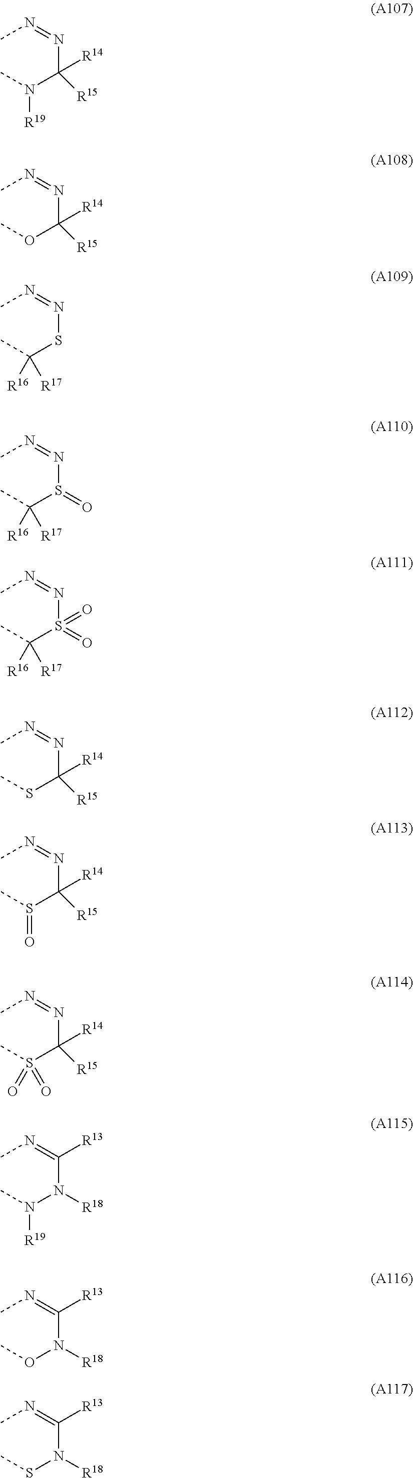 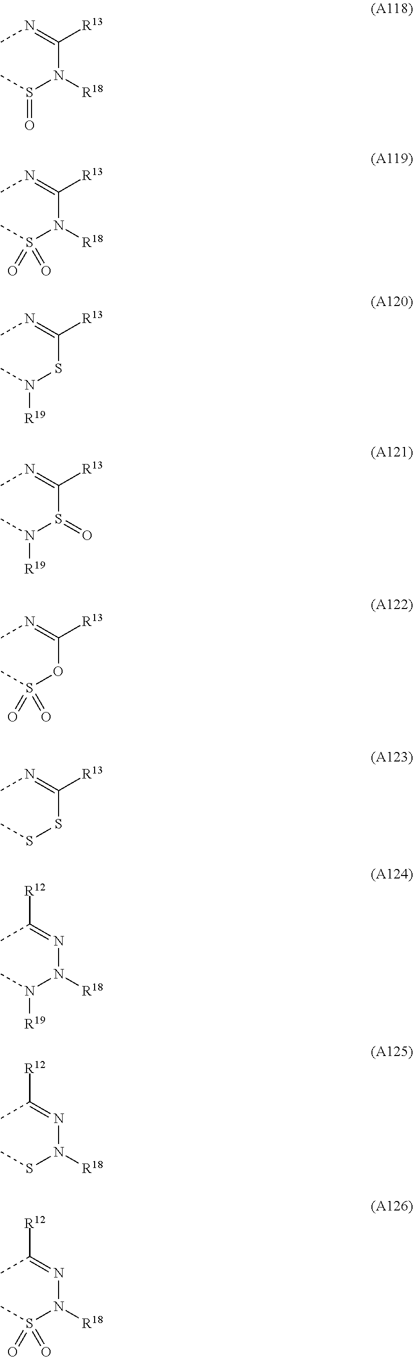 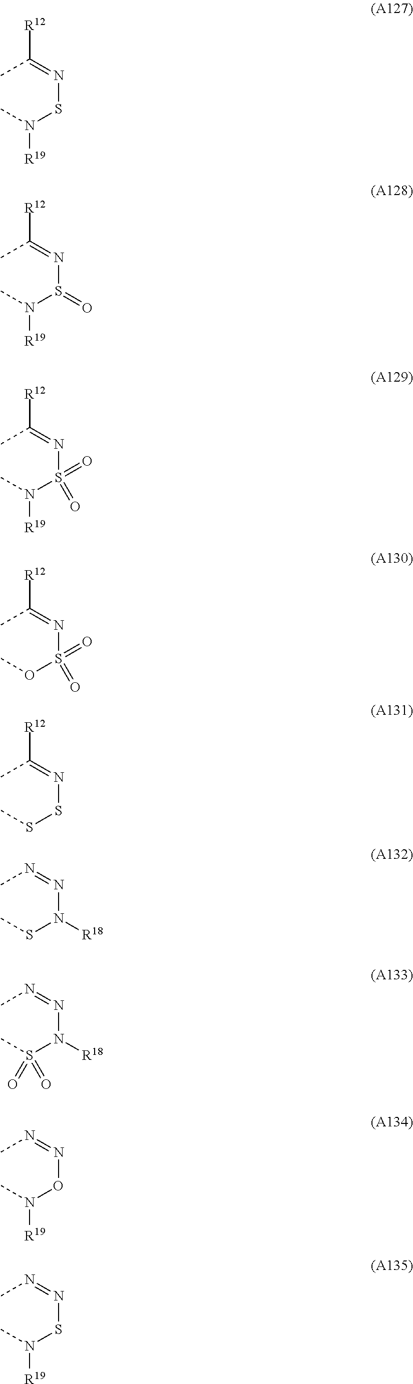 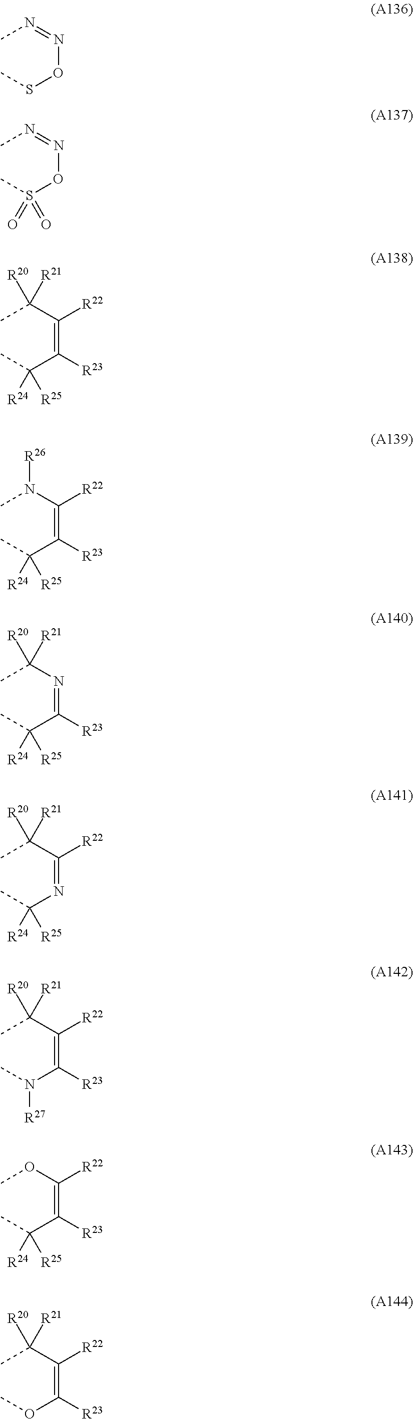 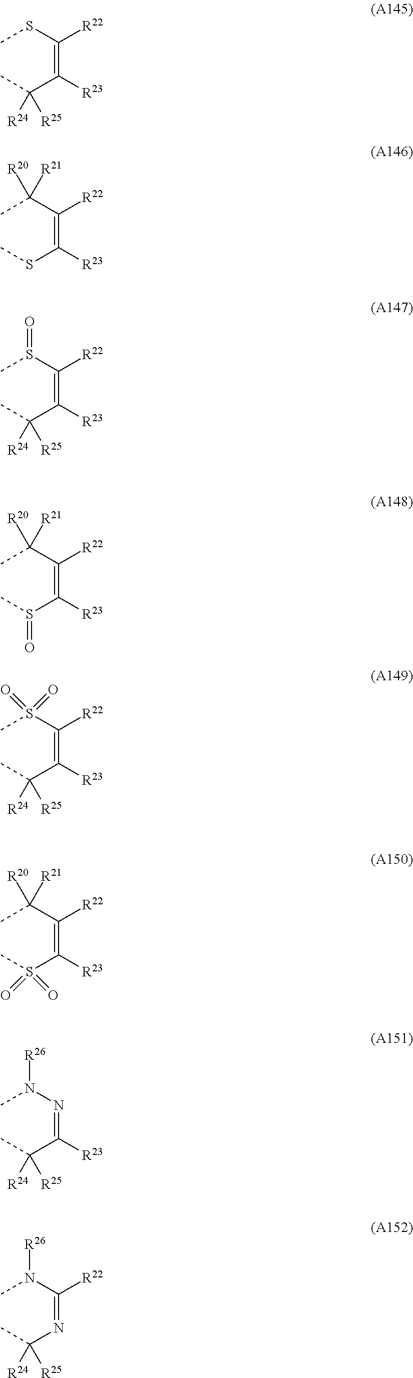 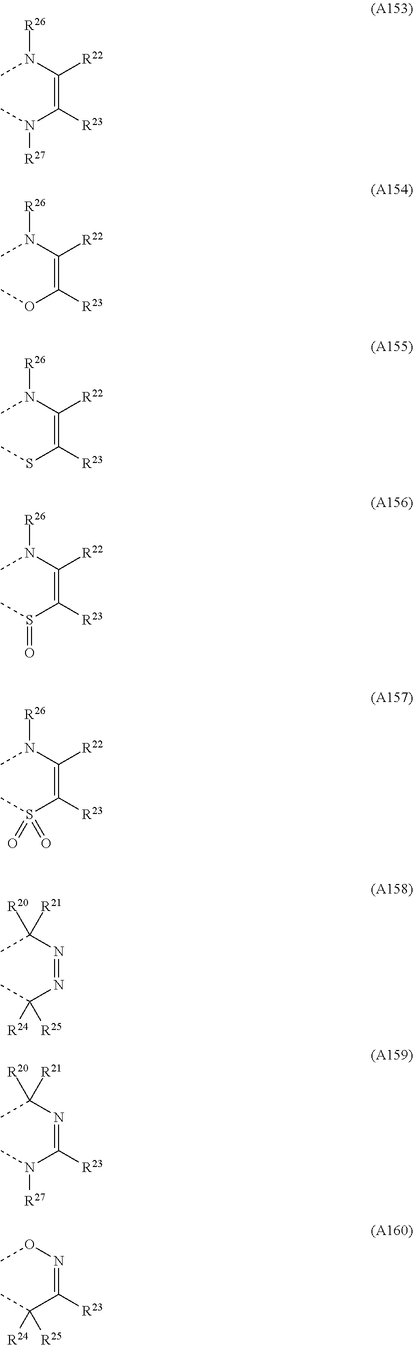 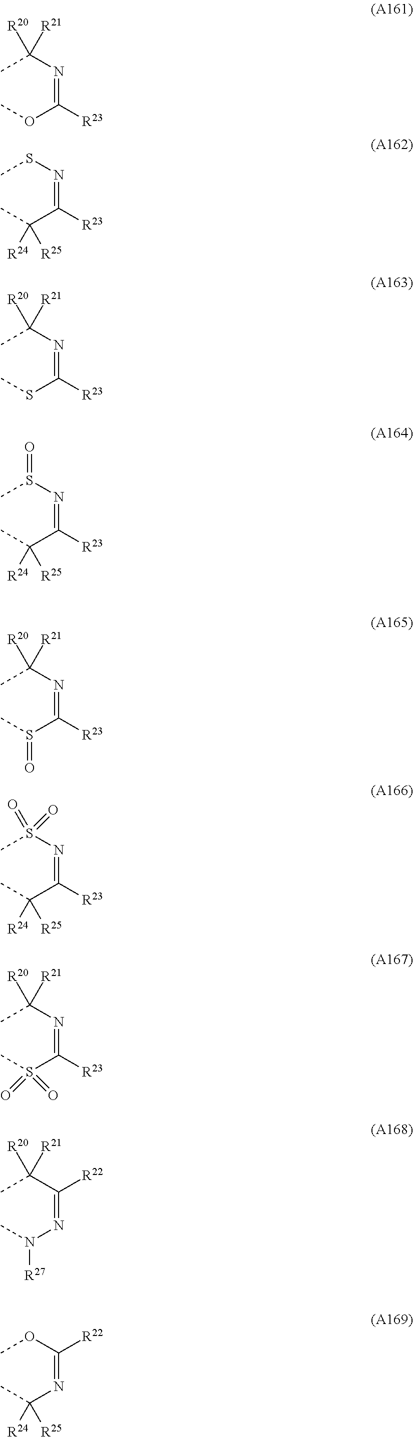 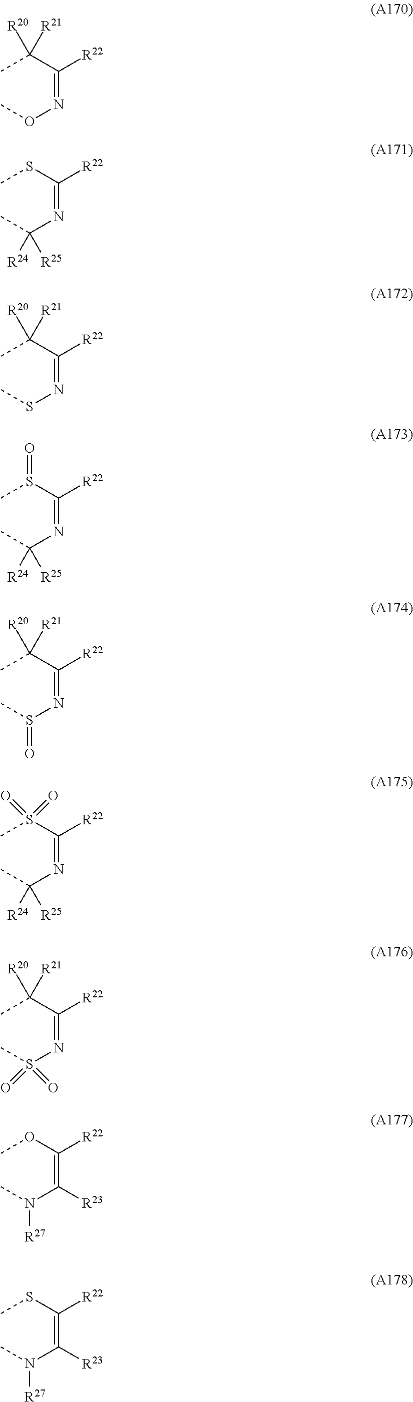 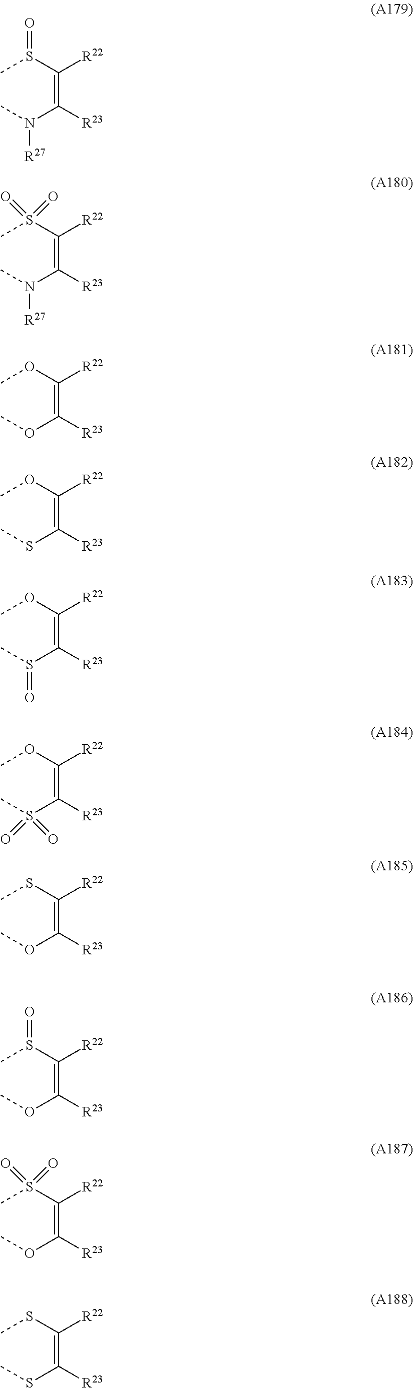 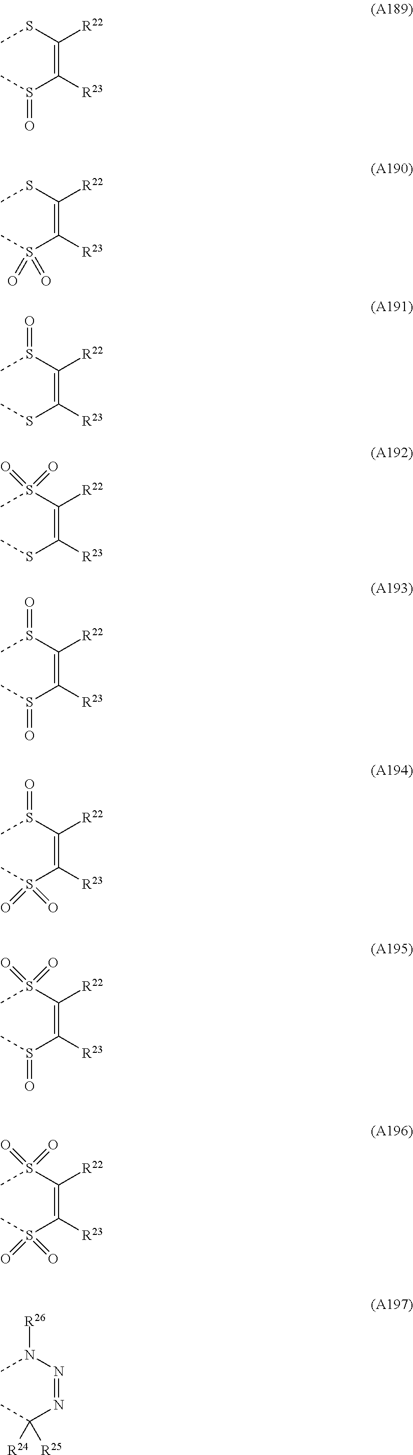 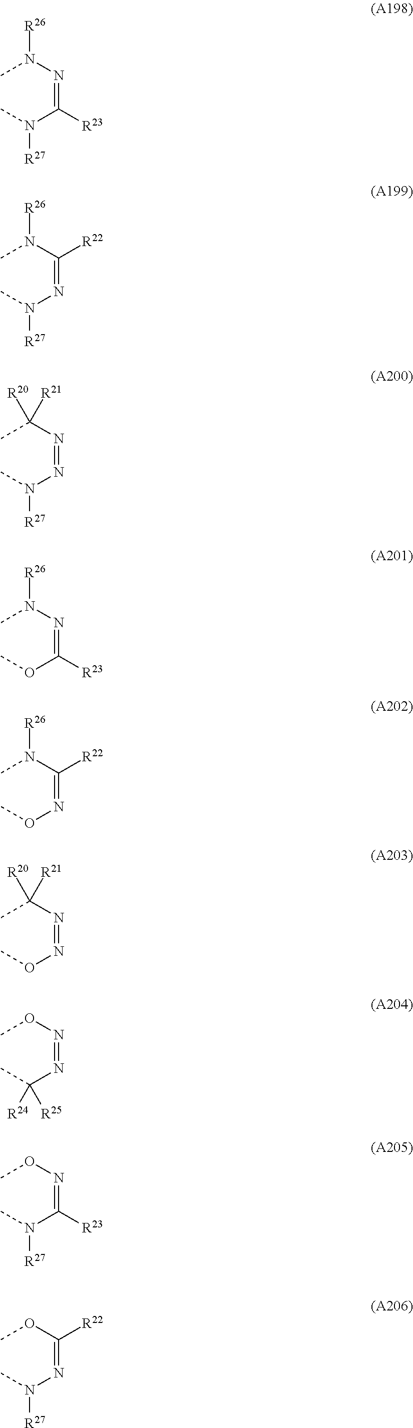 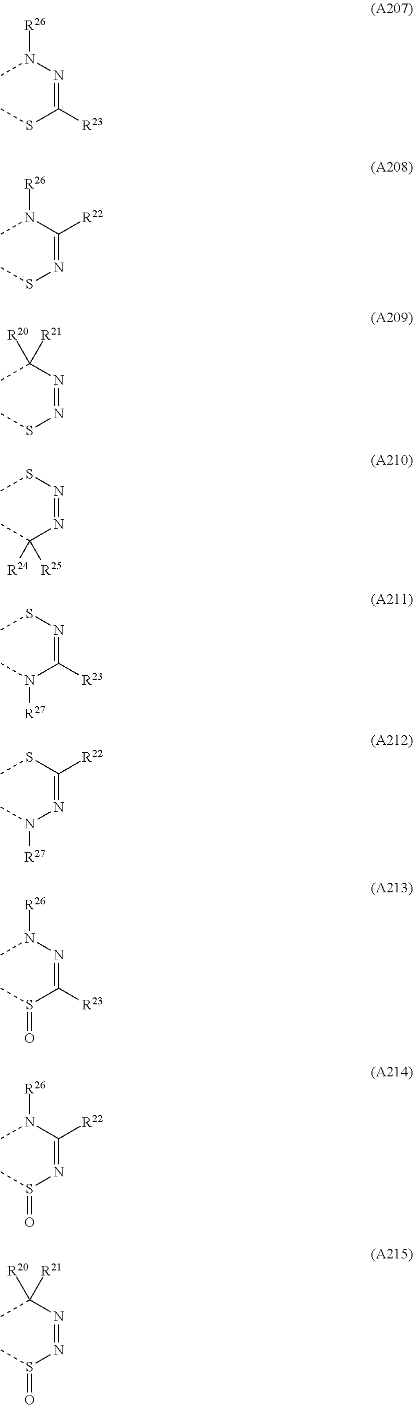 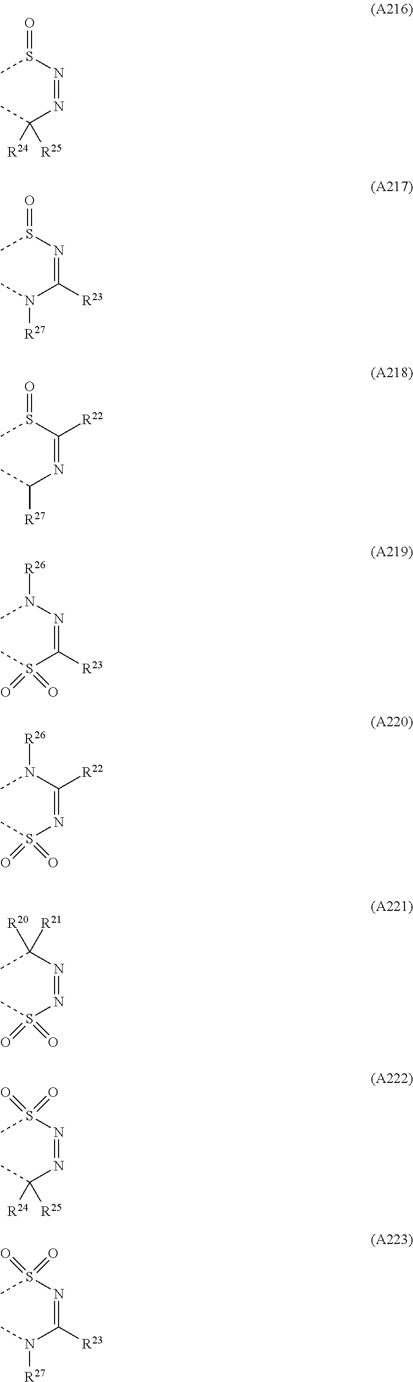 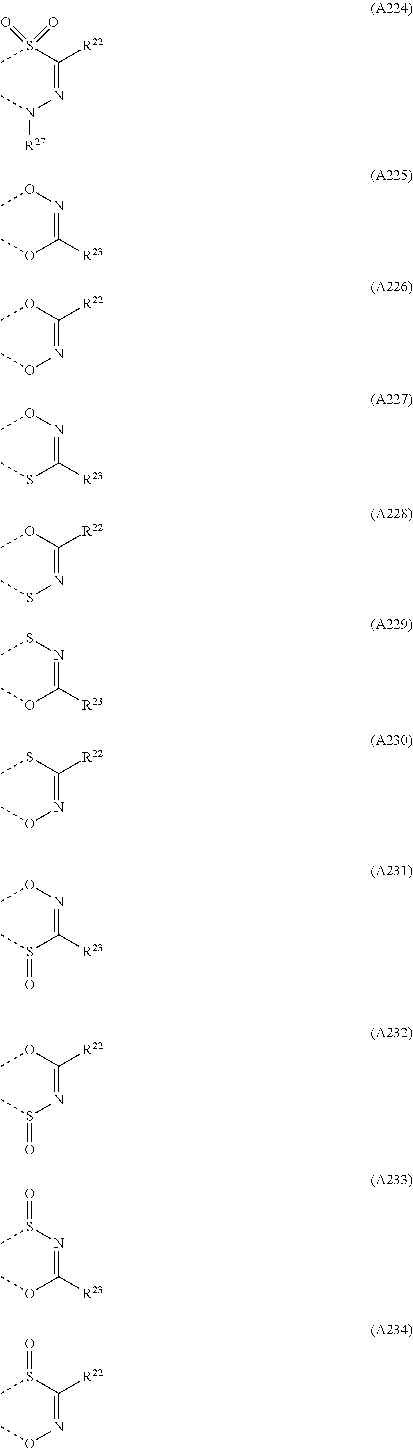 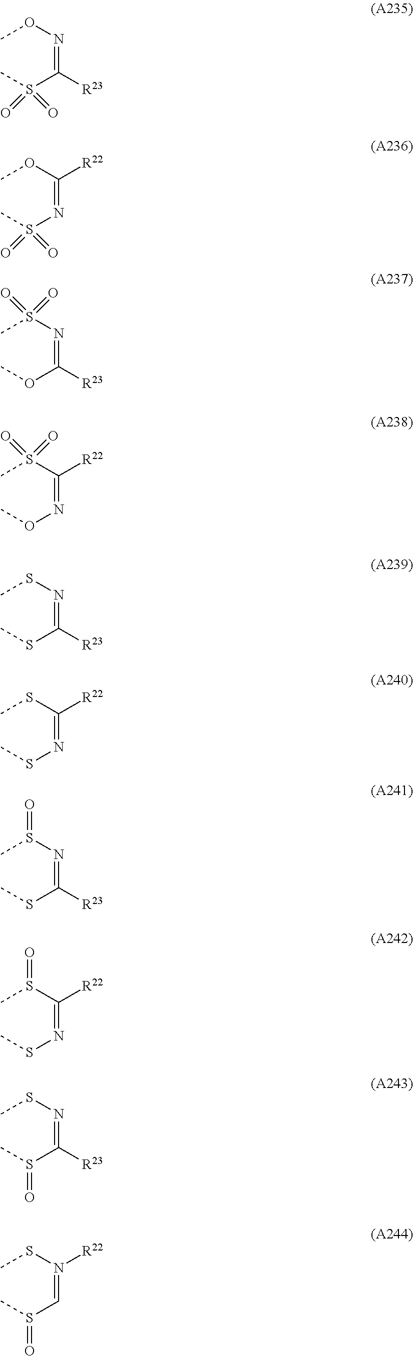 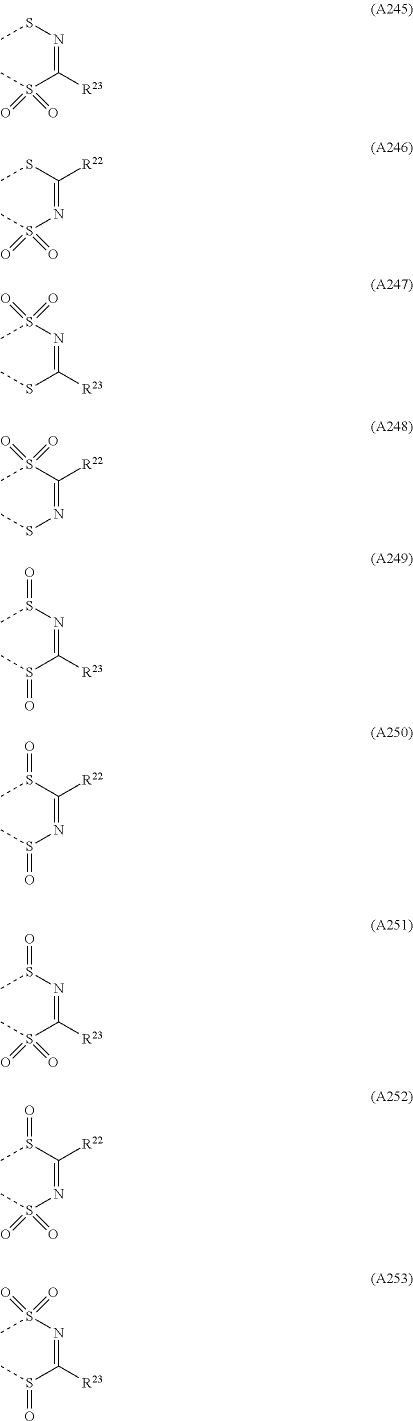 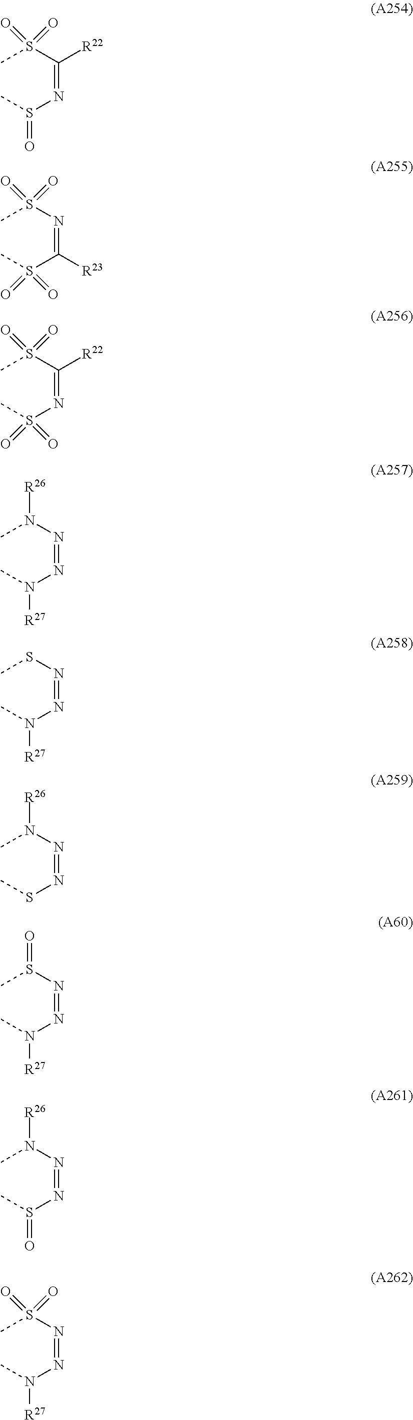 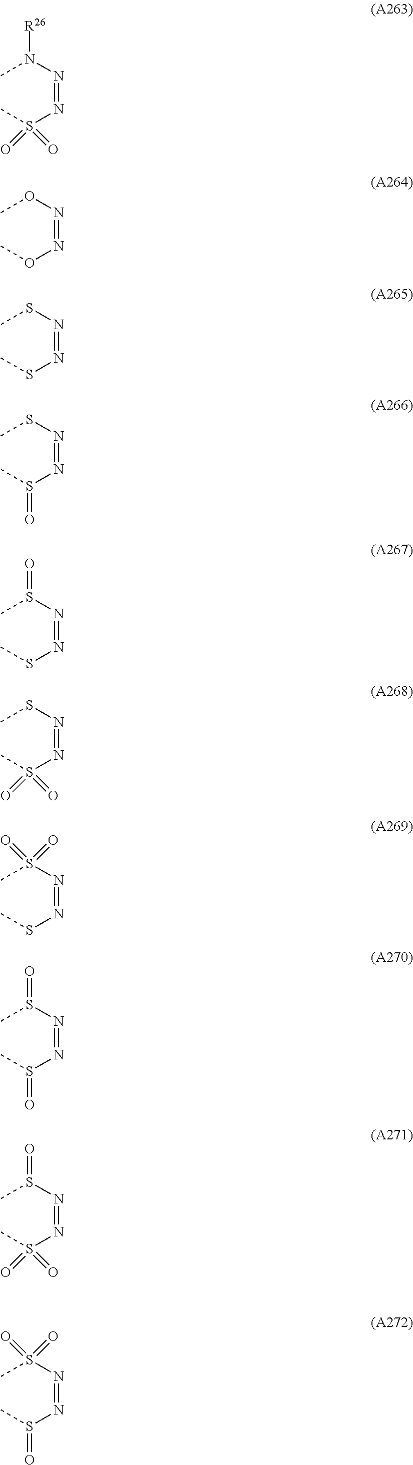 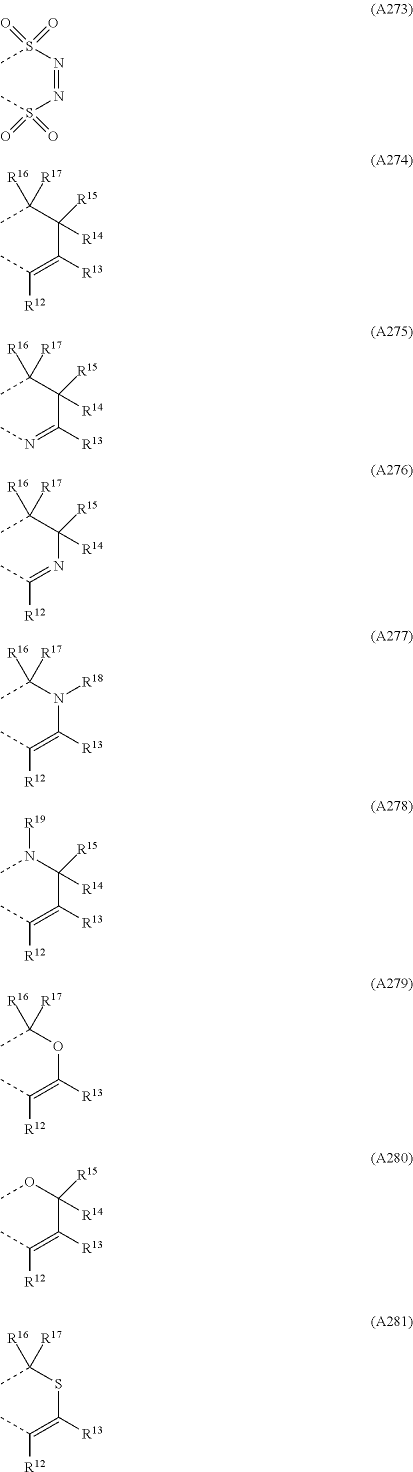 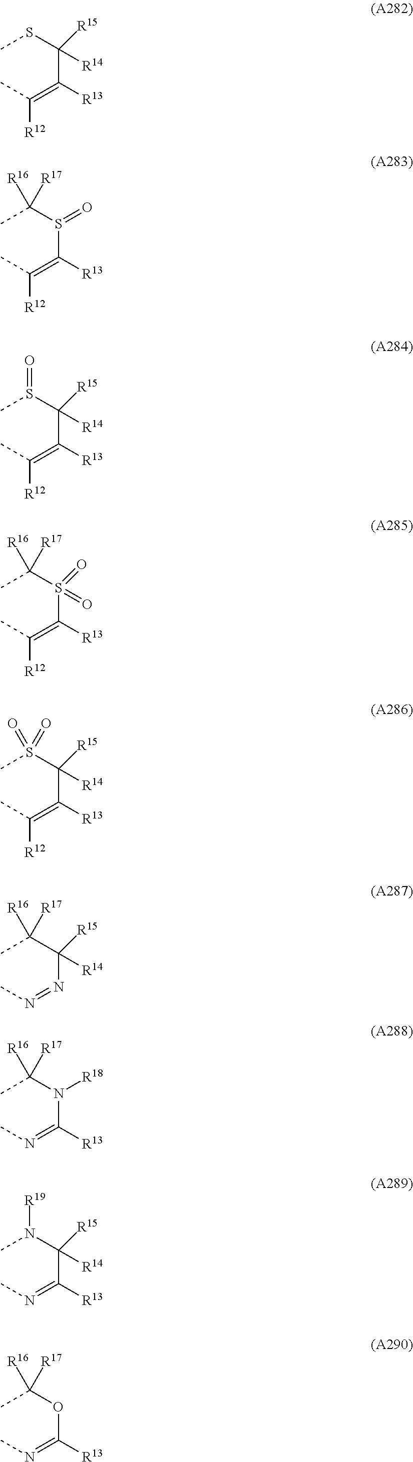 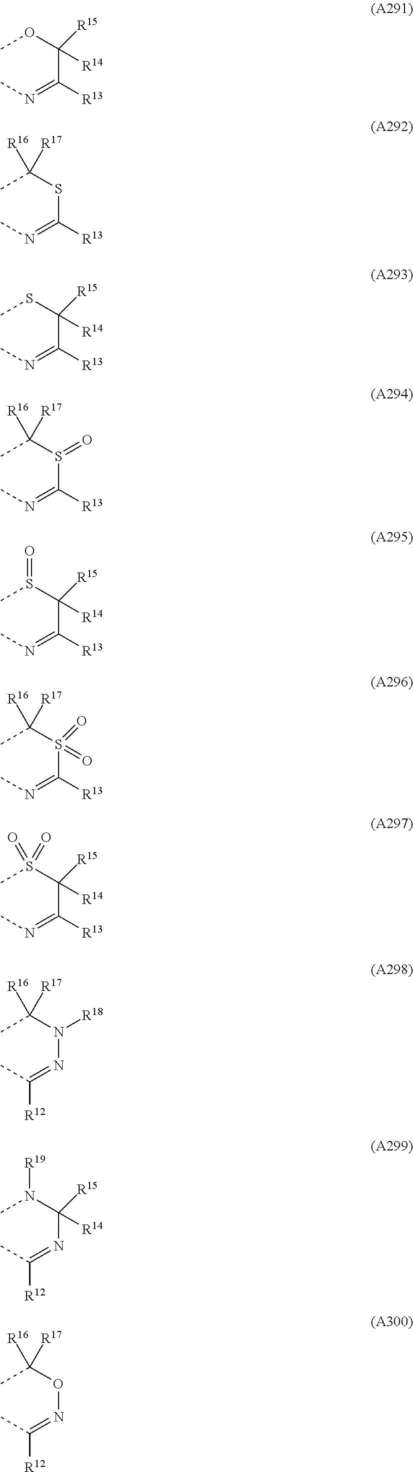 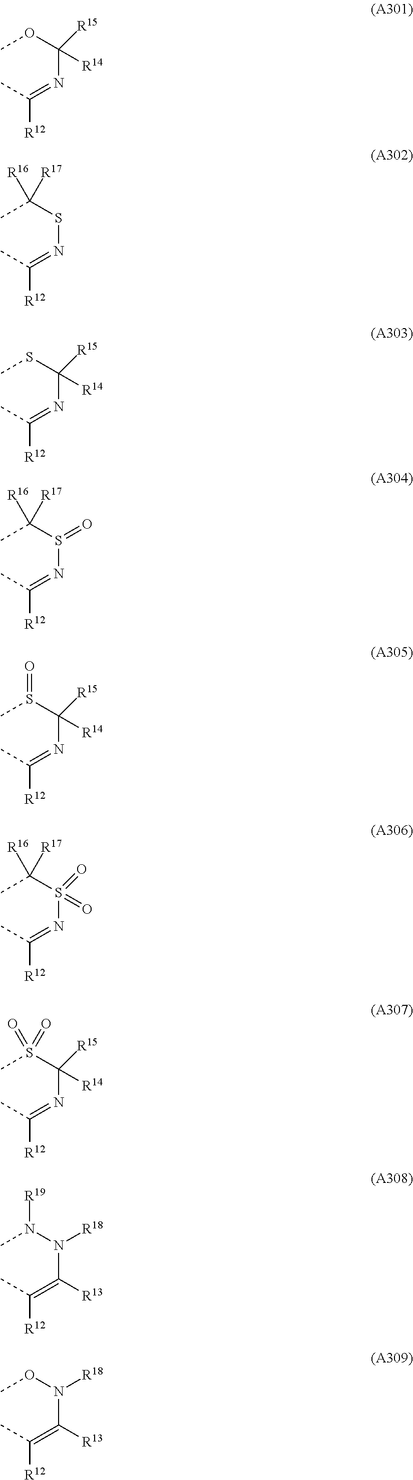 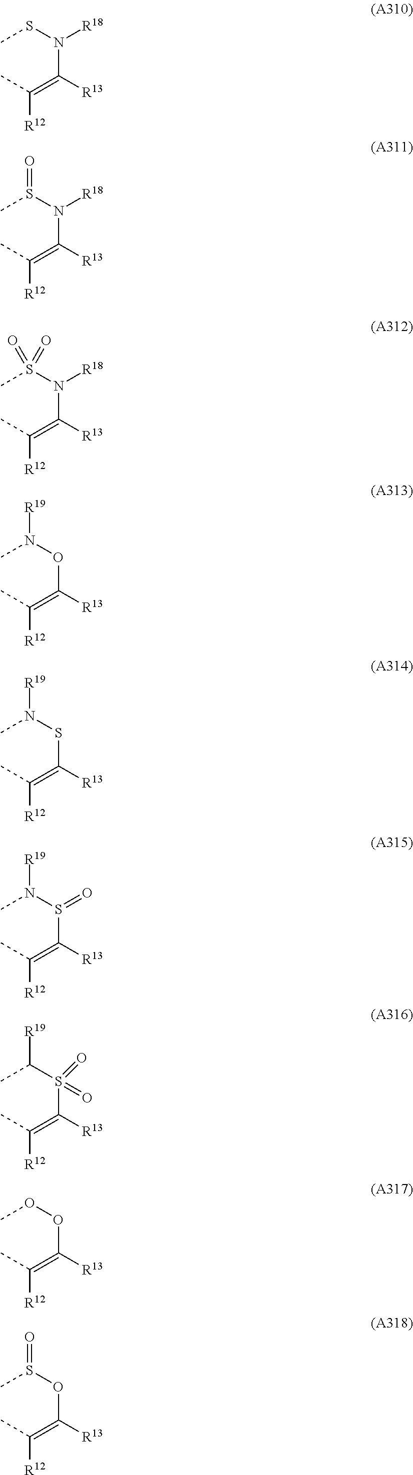 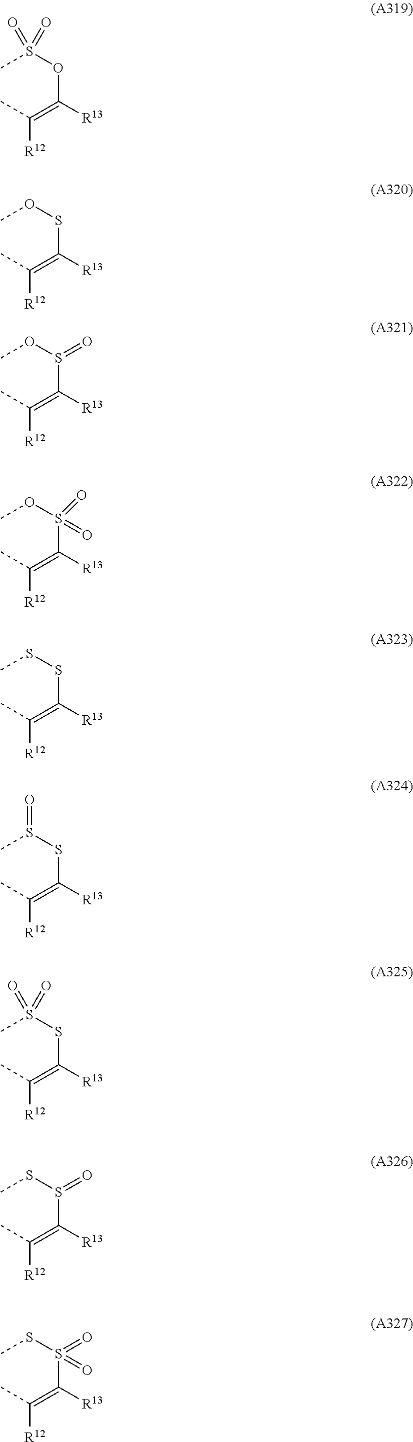 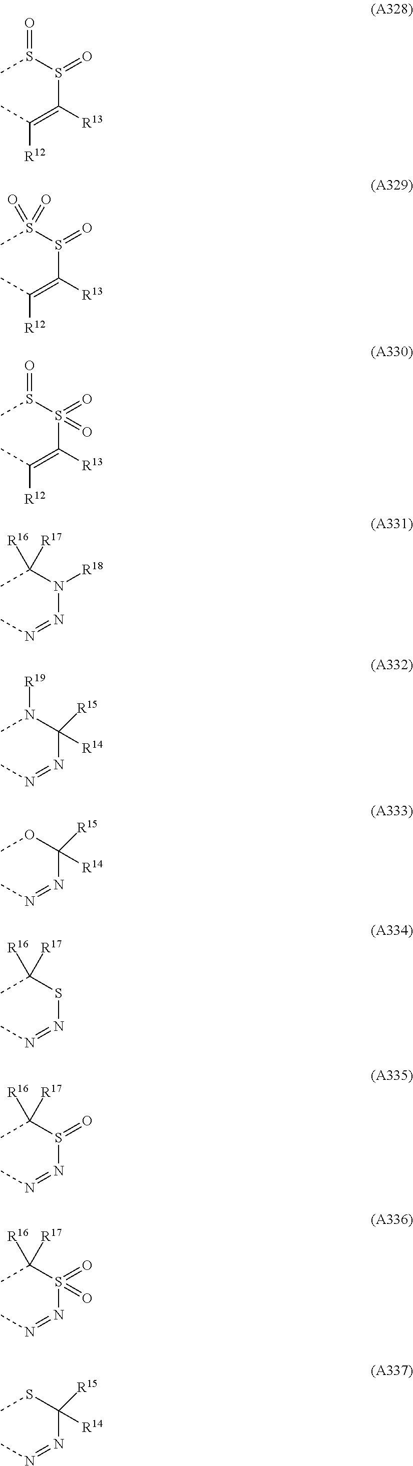 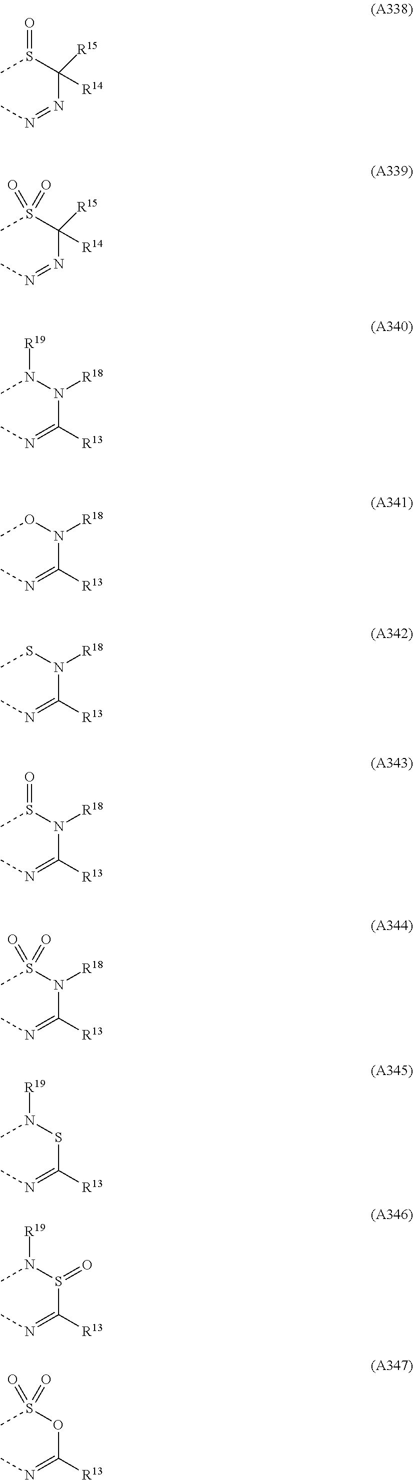 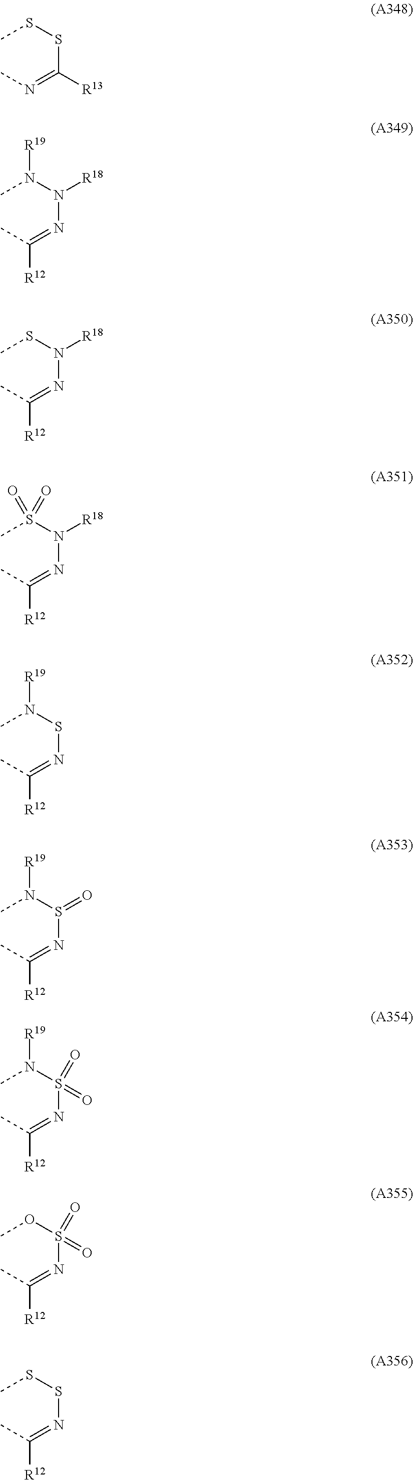 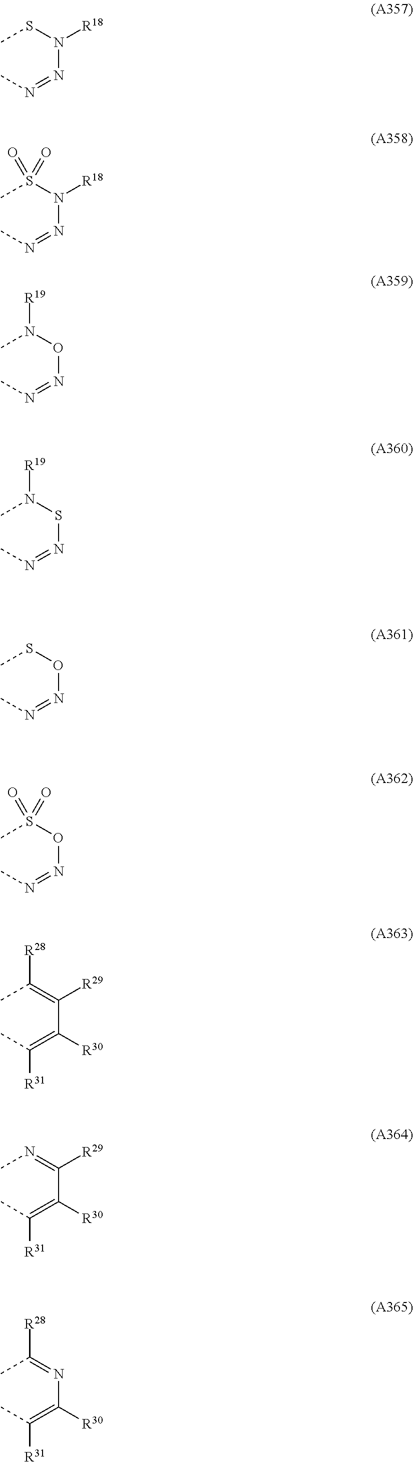 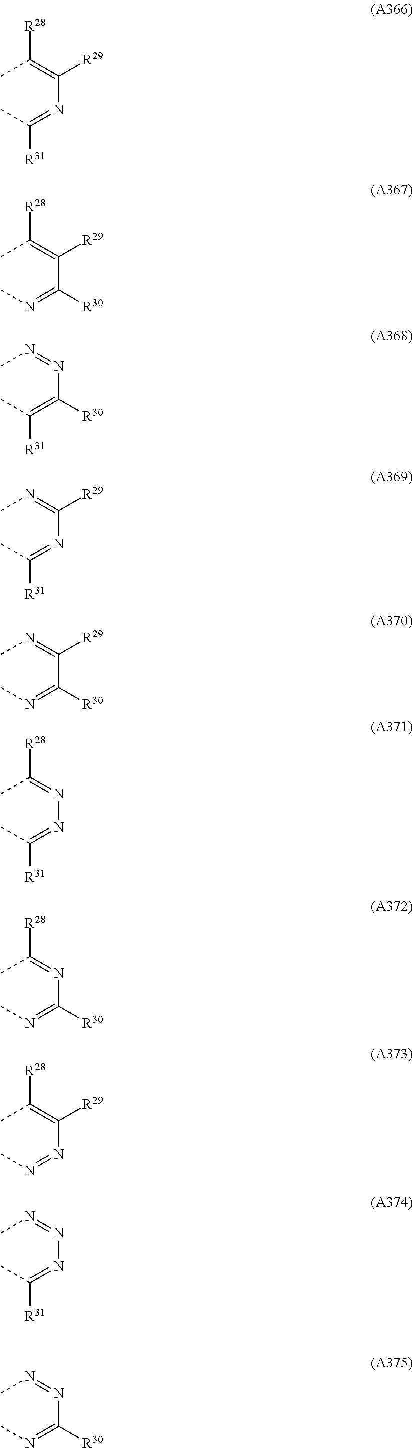 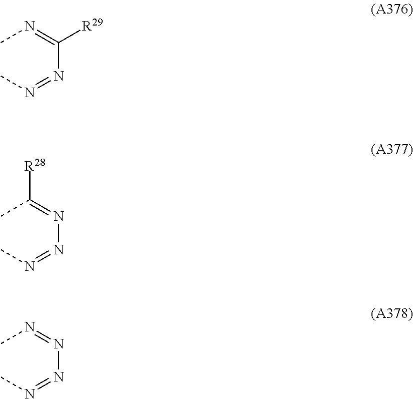
R⁷, R⁸, R¹², R¹³, R²² and R²³ are each independently hydrogen, halogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R²O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups,
R⁹, R¹⁰, R¹⁴, R¹⁵, R¹⁶, R¹⁷, R²⁰, R²¹, R²⁴ and R²⁵ are each independently hydrogen, halogen, (C₁-C₄)-alkyl, (C₁-C₄)-haloalkyl, (C₁-C₄)-alkoxy, (C₁-C₄)-haloalkoxy or (C₁-C₄)-alkoxy-(C₁-C₄)-alkyl or
any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or
any two geminal R⁹, R¹⁰, R¹⁴, R¹⁵, R¹⁶, R¹⁷, R²⁰, R²¹, R²⁴ and R²⁵ are an acetal of the formula —O—(C₂-C₄)-alkylene-O—,
R¹¹, R¹⁸, R¹⁹, R²⁶ ad R²⁷ are each independently hydrogen, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of nitro, cyano, R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,
or R¹¹, R¹⁸, R¹⁹, R²⁶ and R²⁷ are each independently (C₃-C₇)-cycloalkyl, heteroaryl, heterocyclyl or phenyl, where the four aforementioned radicals are each substituted by s radicals from the group consisting of halogen, nitro, cyano, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl, and where heterocyclyl bears n oxo groups,
R²⁸, R²⁹, R³⁰ and R³¹ are each independently hydrogen, nitro, halogen, cyano, thiocyanato, (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkenyl, halo-(C₃-C₆)-cycloalkenyl, (C₃-C₆)-cycloalkyl-(C₁-C₆)-alkyl, halo-(C₃-C₆)-cycloalkyl-(C₁-C₆)-alkyl, (C₃-C₆)-cycloalkenyl-(C₁-C₆)-alkyl, halo-(C₃-C₆)-cycloalkenyl-(C₁-C₆)-alkyl, R¹(O)C, R¹(R¹ON═)C, R¹O(O)C, (R¹)₂N(O)C, R¹(R¹O)N(O)C, (R¹)₂N(R¹)N(O)C, R¹(O)C(R¹)N(O)C, R²O(O)C(R¹)N(O)C, (R¹)₂N(O)C(R¹)N(O)C, R²(O)₂S(R¹)N(O)C, R¹O(O)₂S(R¹)N(O)C, (R¹)₂N(O)₂S(R¹)N(O)C, R²O, R¹(O)CO, R²(O)₂SO, R²O(O)CO, (R¹)₂N(O)CO, (R¹)₂N, R¹(O)C(R¹)N, R²(O)₂S(R¹)N, R²O(O)C(R¹)N, (R¹)₂N(O)C(R¹)N, R¹O(O)₂S(R¹)N, (R¹)₂N(O)₂S(R¹)N, R²(O)ₙS, R¹O(O)₂S, (R¹)₂N(O)₂S, R¹(O)C(R¹)N(O)₂S, R²O(O)C(R¹)N(O)₂S, (R¹)₂N(O)C(R¹)N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, (R¹O)(R¹)N(O)C—(C₁-C₆)-alkyl, (R¹)₂N(R¹)N(O)C—(C₁-C₆)-alkyl, R¹(O)C(R¹)N(O)C—(C₁-C₆)-alkyl, R²O(O)C(R¹)N(O)C—(C₁-C₆)-alkyl, (R¹)₂N(O)C(R¹)N(O)C—(C₁-C₆)-alkyl, R²(O)₂S(R¹)N(O)C—(C₁-C₆)-alkyl, R¹O(O)₂S(R¹)N(O)C—(C₁-C₆)-alkyl, (R¹)₂N(O)₂S(R¹)N(O)C—(C₁-C₆)-alkyl, NC—(C₁-C₆)-alkyl, R¹O—(C₁-C₆)-alkyl, R¹(O)CO—(C₁-C₆)-alkyl, R²(O)₂SO—(C₁-C₆)-alkyl, R²O(O)CO—(C₁-C₆)-alkyl, (R¹)₂N(O)CO—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R¹O(O)₂S(R¹)N—(C₁-C₆)-alkyl, (R¹)₂N(O)₂S(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, R¹(O)C(R¹)N(O)₂S—(C₁-C₆)-alkyl, R²O(O)C(R¹)N(O)₂S—(C₁-C₆)-alkyl, (R¹)₂N(O)C(R¹)N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups.

3. An N-(tetrazol-5-yl)-, N-(triazol-5-yl)-, N-(1,2,5-oxadiazol-3-yl)- or N-(1,3,4-oxadiazol-2-yl)bicycloarylcarboxamide as claimed in claim 1, in which
Q is a Q1, Q2, Q3 or Q4 radical
X is nitro, halogen, cyano, (C₁-C₆)-alkyl, halo-(C₁-C₆)-alkyl, (C₂-C₆)-alkenyl, (C₂-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, R¹(O)C, R¹(R¹ON═)C, R¹O(O)C, (R¹)₂N(O)C, R¹O, (R¹)₂N, R¹(O)C(R¹)N, R²(O)₂S(R¹)N, R²O(O)C(R¹)N, (R¹)₂N(O)C(R¹)N, R²(O)ₙS, R¹O(O)₂S, (R¹)₂N(O)₂S, (R⁵O)₂(O)P, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, (R¹)₂N—(C₁-C₆)-alkyl, R¹(O)C(R¹)N—(C₁-C₆)-alkyl, R²(O)₂S(R¹)N—(C₁-C₆)-alkyl, R²O(O)C(R¹)N—(C₁-C₆)-alkyl, (R¹)₂N(O)C(R¹)N—(C₁-C₆)-alkyl, R²(O)ₙS—(C₁-C₆)-alkyl, R¹O(O)₂S—(C₁-C₆)-alkyl, (R¹)₂N(O)₂S—(C₁-C₆)-alkyl, (R⁵O)₂(O)P—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, thiocyanato, (C₁-C₆)-alkyl, halo-(C₁-C₆)-alkyl, R¹O, (R¹)₂N, R²(O)ₙS, R¹O(O)₂S, (R¹)₂N(O)₂S and R¹O—(C₁-C₆)-alkyl, and where heterocyclyl bears n oxo groups,
W is hydrogen, halogen, nitro, cyano, (C₁-C₆)-alkyl, halo-(C₁-C₆)-alkyl, (C₃-C₇)-cycloalkyl, (C₁-C₆)-alkoxy, (C₁-C₆)-alkyl-(O)ₙS—, R¹O(O)C, (R¹)₂N, R¹(O)C(R¹)N or R²(O)₂S(R¹)N,
R is hydrogen,
R^{X} is (C₁-C₆)-alkyl, halo-(C₁-C₆)-alkyl, (C₂-C₆)-alkenyl, halo-(C₂-C₆)-alkenyl, (C₂-C₆)-alkynyl, halo-(C₃-C₆)-alkynyl, where the six aforementioned radicals are each substituted by s radicals from the group consisting of R²(O)ₙS, (R¹)₂N, R¹O, R¹(O)C, R¹O(O)C, R¹(O)CO, R²O(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy and halogen, and where heterocyclyl bears n oxo groups,
or R^{X} is (C₃-C₇)-cycloalkyl, where this radical is substituted by s radicals from the group consisting of halogen, (C₁-C₆)-alkyl and halo-(C₁-C₆)-alkyl,
R^{Y} is hydrogen, (C₁-C₆)-alkyl, halo-(C₁-C₆)-alkyl, (C₃-C₇)-cycloalkyl, (C₁-C₆)-alkoxy, methoxycarbonyl, methoxycarbonylmethyl, halogen, amino, aminocarbonyl or methoxymethyl,
R^{Z} is hydrogen, (C₁-C₆)-alkyl, R¹O—(C₁-C₆)-alkyl, R′CH₂, (C₃-C₇)-cycloalkyl, halo-(C₁-C₆)-alkyl, R¹O, R¹(H)N, methoxycarbonyl, acetylamino or methylsulfonyl,
R¹ is hydrogen, (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine latter radicals are each substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl bears n oxo groups,
R² is (C₁-C₆)-alkyl, halo-(C₁-C₆)-alkyl, (C₃-C₆)-cycloalkyl, halo-(C₃-C₆)-cycloalkyl, (C₃-C₆)-cycloalkyl-(C₁-C₆)-alkyl, (C₁-C₆)-alkyl-O—(C₁-C₆)-alkyl, cycloalkyl-(C₁-C₆)-alkyl-O—(C₁-C₆)-alkyl, phenyl, phenyl-(C₁-C₆)-alkyl, heteroaryl, heteroaryl-(C₁-C₆)-alkyl, heterocyclyl, heterocyclyl-(C₁-C₆)-alkyl, phenyl-O—(C₁-C₆)-alkyl, heteroaryl-O—(C₁-C₆)-alkyl, heterocyclyl-O—(C₁-C₆)-alkyl, where the nine latter radicals are each substituted by s radicals from the group consisting of nitro, halogen, (C₁-C₆)-alkyl, halo-(C₁-C₆)-alkyl, R³O(O)C, (R³)₂N(O)C, R³O, (R³)₂N, R⁴(O)ₙS and R³O—(C₁-C₆)-alkyl, and where heterocyclyl bears n oxo groups,
R³ is hydrogen or (C₁-C₆)-alkyl,
R⁴ is (C₁-C₆)-alkyl,
R⁵ is hydrogen or (C₁-C₄)-alkyl,
R′ is acetoxy, acetamido, methoxycarbonyl or (C₃-C₆)-cycloalkyl,
n is 0, 1 or 2,
s is 0, 1, 2 or 3,
L is a bridge selected from the group consisting of A1, A2, A3, A4, A5, A6, A7, A8, A9, A10, A11, A12, A13, A14, A17, A25, A26, A27, A28, A29, A30, A31, A32, A33, A34, A35, A36, A37, A38, A41, A49, A50, A51, A53, A55, A57, A59, A61, A62, A72, A139, A140, A141, A142, A143, A144, A145, A146, A147, A148, A149, A150, A151, A157, A158, A168, A274, A275, A276, A277, A278, A279, A280, A281, A282, A283, A284, A285, A286, A287, A363, A364, A365, A366, A367, A368, A369, A370, A371, A372 and A373,
R⁷, R⁸, R¹², R¹³, R²² and R²³ are each independently hydrogen, halogen, (C₁-C₆)-alkyl, halo-(C₁-C₆)-alkyl, (C₃-C₆)-cycloalkyl, halo-(C₃-C₆)-cycloalkyl, R¹(O)C, R¹(R¹ON═)C, R¹O(O)C, (R¹)₂N(O)C, R²O, R¹(O)CO, (R¹)₂N, R¹(O)C(R¹)N, R²(O)ₙS, R¹O—(C₁-C₆)-alkyl or R²(O)ₙS—(C₁-C₆)-alkyl,
R⁹, R¹⁰, R¹⁴, R¹⁵, R¹⁶, R¹⁷, R²⁰, R²¹, R²⁴ and R²⁵ are each independently hydrogen, halogen, (C₁-C₄)-alkyl, (C₁-C₄)-haloalkyl or (C₁-C₄)-alkoxy, or
any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or
any two geminal R⁹, R¹⁰, R¹⁴, R¹⁵, R¹⁶, R¹⁷, R²⁰, R²¹, R²⁴ and R²⁵ are an acetal of the formula —O—(C₂-C₄)-alkylene-O—,
R¹¹, R¹⁸, R¹⁹, R²⁶ and R²⁷ each independently hydrogen or (C₁-C₆)-alkyl, where the (C₁-C₆)-alkyl group is substituted by s radicals from the group consisting of R²(O)ₙS, (R¹)₂N, R¹O, R¹(O)C, R¹O(O)C, R¹(O)CO, R¹(O)CO, R¹(O)C(R¹)N, R²(O)₂S(R¹)N, (C₃-C₆)-cycloalkyl, heteroaryl, heterocyclyl and phenyl, where the four latter radicals are substituted by s radicals from the group consisting of (C₁-C₆)-alkyl, halo-(C₁-C₆)-alkyl, (C₁-C₆)-alkoxy, halo-(C₁-C₆)-alkoxy and halogen, and where heterocyclyl bears n oxo groups,
or R¹¹, R¹⁸, R¹⁹, R²⁶ and R²⁷ are each independently (C₃-C₇)-cycloalkyl, heteroaryl, heterocyclyl or phenyl, where the four aforementioned radicals are each substituted by s radicals from the group consisting of halogen, nitro, (C₁-C₆)-alkyl, halo-(C₁-C₆)-alkyl, (C₃-C₆)-cycloalkyl, (C₁-C₆)-alkyl-S(O)ₙ, (C₁-C₆)-alkoxy, halo-(C₁-C₆)-alkoxy and (C₁-C₆)-alkoxy-(C₁-C₄)-alkyl,
R²⁸, R²⁹, R³⁰ and R³¹ are each independently hydrogen, nitro, halogen, cyano, (C₁-C₆)-alkyl, halo-(C₁-C₆)-alkyl, (C₃-C₆)-cycloalkyl, halo-(C₃-C₆)-cycloalkyl, R¹(O)C, R¹(R¹ON═)C, R¹O(O)C, (R¹)₂N(O)C, R²O, R¹(O)CO, (R¹)₂N, R¹(O)C(R¹)N, R²(O)ₙS, R¹O(O)₂S, R¹(O)C—(C₁-C₆)-alkyl, R¹O(O)C—(C₁-C₆)-alkyl, (R¹)₂N(O)C—(C₁-C₆)-alkyl, NC—(C₁-C₆)-alkyl, R¹O—(C₁-C₆)-alkyl, R¹(O)CO—(C₁-C₆)-alkyl, (R¹)₂N—(C₁-C₆)-alkyl, R¹(O)C(R¹)N—(C₁-C₆)-alkyl, R²(O)ₙS—(C₁-C₆)-alkyl, phenyl, heteroaryl, heterocyclyl, phenyl-(C₁-C₆)-alkyl, heteroaryl-(C₁-C₆)-alkyl, heterocyclyl-(C₁-C₆)-alkyl, where the six latter radicals are each substituted by s radicals from the group consisting of nitro, halogen, cyano, (C₁-C₆)-alkyl, halo-(C₁-C₆)-alkyl, (C₃-C₆)-cycloalkyl, R¹O(O)C, (R¹)₂N(O)C, R¹O, (R¹)₂N, R²(O)ₙS, R¹O(O)₂S, (R¹)₂N(O)₂S and R¹O—(C₁-C₆)-alkyl, and where heterocyclyl bears n oxo groups.

4. An N-(tetrazol-5-yl)-, N-(triazol-5-yl)-, N-(1,2,5-oxadiazol-3-yl)- or N-(1,3,4-oxadiazol-2-yl)bicycloarylcarboxamide as claimed in claim 1, in which
Q is a Q1, Q2, Q3 or Q4 radical
X is nitro, halogen, methyl, ethyl, n-propyl, isopropyl, trifluoromethyl, difluoromethyl, chlorodifluoromethyl, dichlorofluoromethyl, trichloromethyl, pentafluoroethyl, heptafluoroisopropyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl,
W is hydrogen, chlorine or methyl,
R is hydrogen,
R^{X} is methyl, ethyl, n-propyl, prop-2-en-1-yl, methoxyethyl, ethoxyethyl or methoxyethoxyethyl,
R^{Y} is methyl, ethyl, n-propyl, chlorine or amino,
R^{Z} is methyl, ethyl, n-propyl or methoxymethyl.
L is a bridge selected from the group consisting of A1, A2, A4, A5, A6, A7, A8, A25, A26, A28, A29, A30, A31, A32, A49, A50, A51, A53, ASS, A57, A59, A61, A139, A140, A141, A142, A143, A145, A146, A147, A148, A149, A150, A274, A275, A278, A279, A280, A281, A282, A283, A284, A285, A286, A363, A364, A365, A366, A367, A368, A369, A370, A371, A372 and A373,
R⁷, R⁸, R¹², R¹³, R²² and R²³ are each independently hydrogen, halogen, methyl, ethyl, n-propyl, isopropyl, trifluoromethyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl,
R⁹, R¹⁰, R¹⁴, R¹⁵, R¹⁶, R¹⁷, R²⁰, R²¹, R²⁴ and R²⁵ are each independently hydrogen, halogen, methyl, methoxy, ethoxy or
any two geminal R⁹, R¹⁰, R¹⁴, R¹⁵, R¹⁶, R¹⁷, R²⁰, R²¹, R²⁴ and R²⁵ together with the carbon atom to which they are bonded form a carbonyl group or an oxime of the formula C═NOR¹ or
any two geminal R⁹, R¹⁰, R¹⁴, R¹⁵, R¹⁶, R¹⁷, R²⁰, R²¹, R²⁴ and R²⁵ are an acetal of the formula —O—(CH₂)₂—O—,
R¹ is hydrogen, methyl or ethyl,
R¹¹, R¹⁹, R²⁶ and R²⁷ are each independently hydrogen or methyl,
R²⁸, R²⁹, R³⁰ and R³¹ are each independently hydrogen, nitro, halogen, methyl, ethyl, n-propyl, isopropyl, trifluoromethyl, difluoromethyl, chlorodifluoromethyl, dichlorofluoromethyl, trichloromethyl, pentafluoroethyl, heptafluoroisopropyl, cyclopropyl, methoxy, ethoxy, methylsulfanyl, methylsulfinyl, methylsulfonyl, methoxymethyl, ethoxymethyl, methoxyethyl, methoxyethoxymethyl, methylthiomethyl, methylsulfinylmethyl or methylsulfonylmethyl.

5. A herbicidal composition, comprising a herbicidally active content of at least one compound of formula (I) and/or salt as claimed in claim 1.

6. The herbicidal composition as claimed in claim 5 in a mixture with one or more formulation auxiliaries.

7. The herbicidal composition as claimed in claim 5, comprising at least one further pesticidally active substance selected from the group consisting of insecticides, acaricides, herbicides, fungicides, safeners and growth regulators.

8. The herbicidal composition as claimed in claim 7, comprising a safener.

9. The herbicidal composition as claimed in claim 8, comprising cyprosulfamide, cloquintocet-mexyl, mefenpyr-diethyl or isoxadifen-ethyl.

10. The herbicidal composition as claimed in claim 7, comprising a further herbicide.

11. A method for controlling one or more unwanted plants, comprising applying an effective amount of at least one compound as claimed in claim 1 to the plants and/or to a site of unwanted vegetation.

12. A compound as claimed in claim 1 capable of being used for controlling one or more unwanted plants.

13. A compound as claimed in claim 12, capable of being used for controlling unwanted plants in one or more crops of useful plants.

14. A compound as claimed in claim 13, wherein the one or more useful plants are transgenic useful plants.
